(19) United States
(12) Patent Application Publication
(10) Pub. No.: US 2020/0103418 A1
(43) Pub. Date: Apr. 2, 2020

(54) DIAGNOSTIC AND THERAPEUTIC METHODS FOR IRAK4-MEDIATED DISORDERS AND CONDITIONS

(71) Applicant: Genentech, Inc., South San Francisco, CA (US)

(72) Inventors: Jason HACKNEY, San Carlos, CA (US); Alvernia Francesca SETIADI, San Carlos, CA (US); Michael TOWNSEND, San Jose, CA (US); Ali A. ZARRIN, Brisbane, CA (US)

(21) Appl. No.: 16/713,638

(22) Filed: Dec. 13, 2019

Related U.S. Application Data
(63) Continuation of application No. PCT/US2018/037826, filed on Jun. 15, 2018.
(60) Provisional application No. 62/521,299, filed on Jun. 16, 2017.

(51) Int. Cl. G01N 33/68; A61K 31/473; A61K 31/675

(52) U.S. Cl. CPC G01N 33/6893; A61K 31/473; G01N 2800/24; G01N 2800/104; A61K 31/675

(57) ABSTRACT

The present invention provides diagnostic and therapeutic methods and compositions for treating a patient suffering from an interleukin-1 receptor-associated kinase 4 (IRAK4)-mediated disorder or condition, such as an immune disorder (e.g., systemic lupus erythematosus (SLE)) or an inflammatory disorder (e.g., asthma). The invention provides diagnostic methods of monitoring the response of a patient having an IRAK4-mediated disorder or condition to treatment including an IRAK4 pathway inhibitor, methods of identifying a patient having an IRAK4-mediated disorder or condition who may benefit from treatment including an IRAK4 pathway inhibitor, and methods of selecting a therapy for a patient having an IRAK4-mediated disorder or condition based on the expression level of one or more IRAK4 biomarkers (e.g., one or more genes set forth in Table 1). Related therapeutic methods and compositions (e.g., diagnostic kits) are also provided.

### CROSS-REFERENCE TO RELATED APPLICATIONS

This application is a continuation of International Application No. PCT/US2018/037826, filed on Jun. 15, 2018, which claims benefit to U.S. Provisional Application No. 62/521,299, filed on Jun. 16, 2017, the entire disclosures of which are incorporated by reference herein in their entirety.

### SEQUENCE LISTING

The instant application contains a Sequence Listing which has been submitted electronically in ASCII format and is hereby incorporated by reference in its entirety. Said ASCII copy, created on Dec. 9, 2019, is named 50474-170002_Sequence_Listing_12.09.19_ST25 and is 142,799 bytes in size.

### FIELD OF THE INVENTION

The present invention is directed to diagnostic and therapeutic methods for the treatment of interleukin-1 receptor-associated kinase 4 (IRAK4)-mediated disorders or conditions (e.g., immune disorders (e.g., systemic lupus erythematosus (SLE)) or inflammatory disorders (e.g., asthma)) using IRAK4 pathway inhibitors (e.g., an IRAK4 small molecule inhibitor). Also provided are related compositions (e.g., diagnostic kits).

### BACKGROUND

The interleukin-1 receptor-associated kinase (IRAK) family is comprised of four family members IRAK1, IRAK2, IRAK3 (also termed IRAK-M), and IRAK4. These proteins are characterized by a typical N-terminal death domain that mediates interaction with MyD88-family adaptor proteins and a centrally located kinase domain. Whereas IRAK1 and IRAK4 have kinase activity, IRAK2 and IRAK3 are catalytically inactive. Upon activation of their upstream cognate receptors, IRAK4 is thought to phosphorylate IRAK1, resulting in the activation and auto-phosphorylation of IRAK1 and subsequent phosphorylation of downstream substrates. The hyper-phosphorylation of IRAK1 directs its dissociation from the receptor complex and its eventual ubiquitylation and proteasomal degradation. Phosphorylation of downstream substrates such as Pellino-2 ultimately leads to the activation of the MAPKs, such as p38, c-Jun N-terminal kinase (JNK), and NF-kB, followed by production of pro-inflammatory cytokines, chemokines, and destructive enzymes.

The role of IRAK4, in particular, in innate immunity and pathogenesis of autoimmune and inflammatory disorders is emerging. See, e.g., Li et al. PNAS. 99(8): 5567-5572, 2002 and Flannery et al. Biochem. Pharm. 80(12): 1981-1991, 2010. Patients with destabilizing or null mutations in IRAK4 demonstrate defects in toll-like receptor (TLR) signaling and the production of pro-inflammatory cytokines, such as IL-1 and TNF, as well as antiviral cytokines, such as IFNα and IFNβ. These patients demonstrate an increased susceptibility to gram-positive bacterial infections, although they are generally resistant to gram-negative bacterial, viral, and fungal infections. Similarly, IRAK4-deficient mice have defects in TLR- and IL-1-mediated cytokine production and exhibit an increased susceptibility to infection. Not surprisingly, the IRAK4 pathway has been suggested to be involved in various disorders and conditions, including inflammatory, immune-related, and cell proliferative disorders and conditions associated with IRAK-mediated signal transduction, for which there remains an unmet need to develop improved diagnostic methods for identifying patient populations best suited for treatment including an IRAK4 pathway inhibitor (e.g., an IRAK4 small molecule inhibitor).

### SUMMARY OF THE INVENTION

The present invention provides diagnostic methods, therapeutic methods, and kits for the treatment of IRAK4-mediated disorders or conditions (e.g., immune disorders and inflammatory disorders).

In a first aspect, the invention features a method of monitoring the response of a patient having an interleukin-1 receptor-associated kinase 4 (IRAK4)-mediated disorder or condition to treatment comprising an IRAK4 pathway inhibitor, the method comprising: (a) determining, in a sample obtained from the patient at a time point following administration of a first dose of the IRAK4 pathway inhibitor, the expression level of one or more genes set forth in Table 1 (i.e., CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and ACSL1), and (b) comparing the expression level of the one or more genes set forth in Table 1 in the sample with a reference expression level, thereby monitoring the response of the patient to treatment comprising the IRAK4 pathway inhibitor. In some embodiments, the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, PFKFB3, and BCL2A1. In some embodiments, the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, and PFKFB3. In some embodiments, the one or more genes set forth in Table 1 comprises one or more genes selected from the group consisting of CD38, SOCS3, AQP9, CDKN1A, GADD45B, B4GALT5, IL15RA, TNFAIP3, and SOCS1. In some embodiments, the one or more genes set forth in Table 1 comprises all nine of CD38, SOCS3, AQP9, CDKN1A, GADD45B, B4GALT5, IL15RA, TNFAIP3, and SOCS1. In some embodiments, the one or more genes set forth in Table 1 comprises all 11 of CD38, SOCS3, AQP9, CDKN1A, GADD45B, B4GALT5, IL15RA, TNFAIP3, SOCS1, IL1RN, and PFKFB3. In some embodiments, the one or more genes set forth in Table 1 comprises all 12 of CD38, SOCS3, AQP9, CDKN1A, GADD45B, B4GALT5, IL15RA, TNFAIP3, SOCS1, IL1RN, PFKFB3, and BCL2A1. In some embodiments, the one or more genes set forth in Table 1 are all 24 genes set forth in Table 1.

In some embodiments, the expression level of the one or more genes set forth in Table 1 is decreased in the sample obtained from the patient relative to the reference expression level. In some embodiments, the expression level of the one or more genes set forth in Table 1 is decreased at least about 0.5-fold relative to the reference expression level. In some embodiments, the expression level of the one or more genes set forth in Table 1 is decreased at least about 1-fold relative to the reference expression level. In some embodiments, the expression level of the one or more genes set forth in Table 1 is decreased at least about 2-fold relative to the reference expression level. In some embodiments, the expression level of the one or more genes set forth in Table 1 is decreased at least about 3-fold relative to the reference expression level. In some embodiments, the expression level of the one or more genes set forth in Table 1 is decreased at least about 4-fold relative to the reference expression level. In some embodiments, the expression level of the one or more genes set forth in Table 1 is decreased at least about 5-fold relative to the reference expression level. In some embodiments, the expression level of the one or more genes set forth in Table 1 is decreased at least about 10-fold relative to the reference expression level. In some embodiments, the decreased expression level of the one or more genes set forth in Table 1 indicates that the patient is responding to the IRAK4 pathway inhibitor. In some embodiments, the method further comprises administering at least a second dose of an IRAK4 pathway inhibitor to a patient whose expression level of the one or more genes set forth in Table 1 is decreased relative to the reference expression level.

In a second aspect, the invention features a method of treating a patient having an IRAK4-mediated disorder or condition with an IRAK4 pathway inhibitor, the method comprising: (a) determining, in a sample obtained from the patient at a time point following administration of a first dose of the IRAK4 pathway inhibitor, the expression level of one or more genes set forth in Table 1 (i.e., CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and ACSL1), (b) comparing the expression level of the one or more genes set forth in Table 1 in the sample with a reference expression level, and (c) administering at least a second dose of the IRAK4 pathway inhibitor to the patient based on a decreased expression level of the one or more genes set forth in Table 1 relative to the reference expression level. In some embodiments, the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, PFKFB3, and BCL2A1. In some embodiments, the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, and PFKFB3. In some embodiments, the one or more genes set forth in Table 1 comprises one or more genes selected from the group consisting of CD38, SOCS3, AQP9, CDKN1A, GADD45B, B4GALT5, IL15RA, TNFAIP3, and SOCS1. In some embodiments, the one or more genes set forth in Table 1 comprises all nine of CD38, SOCS3, AQP9, CDKN1A, GADD45B, B4GALT5, IL15RA, TNFAIP3, and SOCS1. In some embodiments, the one or more genes set forth in Table 1 comprises all 11 of CD38, SOCS3, AQP9, CDKN1A, GADD45B, B4GALT5, IL15RA, TNFAIP3, SOCS1, IL1RN, and PFKFB3. In some embodiments, the one or more genes set forth in Table 1 comprises all 12 of CD38, SOCS3, AQP9, CDKN1A, GADD45B, B4GALT5, IL15RA, TNFAIP3, SOCS1, IL1RN, PFKFB3, and BCL2A1. In some embodiments, the one or more genes set forth in Table 1 are all 24 genes set forth in Table 1. In some embodiments, the expression level of the one or more genes set forth in Table 1 is decreased at least about 0.5-fold relative to the reference expression level. In some embodiments, the expression level of the one or more genes set forth in Table 1 is decreased at least about 1-fold relative to the reference expression level. In some embodiments, the expression level of the one or more genes set forth in Table 1 is decreased at least about 2-fold relative to the reference expression level. In some embodiments, the expression level of the one or more genes set forth in Table 1 is decreased at least about 3-fold relative to the reference expression level. In some embodiments, the expression level of the one or more genes set forth in Table 1 is decreased at least about 4-fold relative to the reference expression level. In some embodiments, the expression level of the one or more genes set forth in Table 1 is decreased at least about 5-fold relative to the reference expression level. In some embodiments, the expression level of the one or more genes set forth in Table 1 is decreased at least about 10-fold relative to the reference expression level.

In some embodiments of any of the preceding aspects, the reference expression level is: (i) the expression level of the one or more genes set forth in Table 1 in a sample from the patient obtained prior to administration of the first dose of the IRAK4 pathway inhibitor; (ii) the expression level of the one or more genes set forth in Table 1 in a reference population; (iii) a pre-assigned expression level for the one or more genes set forth in Table 1; (iv) the expression level of the one or more genes set forth in Table 1 in a sample obtained from the patient at a previous time point, wherein the previous time point is following administration of the first dose of the IRAK4 pathway inhibitor; or (v) the expression level of the one or more genes set forth in Table 1 in a sample obtained from the patient at a subsequent time point.

In a third aspect, the invention features method of identifying a patient having an IRAK4-mediated disorder or condition who may benefit from treatment comprising an IRAK4 pathway inhibitor, the method comprising determining an expression level of one or more genes set forth in Table 1 (i.e., CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and ACSL1) in a sample obtained from the patient, wherein an increased expression level of the one or more genes set forth in Table 1 in the sample as compared to a reference expression level identifies the patient as one who may benefit from treatment comprising an IRAK4 pathway inhibitor.

In a fourth aspect, the invention features a method of selecting a therapy for a patient having an IRAK4-mediated disorder or condition, the method comprising determining an expression level of one or more genes set forth in Table 1 (i.e., CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and ACSL1) in a sample obtained from the patient, wherein an increased expression level of the one or more genes set forth in Table 1 in the sample as compared to a reference expression level identifies the patient as one who may benefit from treatment comprising an IRAK4 pathway inhibitor.

In some embodiments of the third or fourth aspect, the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, PFKFB3, and BCL2A1. In some embodiments, the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, and PFKFB3. In some embodiments, the one or more genes set forth in Table 1 comprises one or more genes selected from the group consisting of CD38, SOCS3, AQP9, CDKN1A, GADD45B, B4GALT5, IL15RA, TNFAIP3, and SOCS1. In some embodiments, the one or more genes set forth in Table 1 comprises all nine of CD38, SOCS3, AQP9, CDKN1A, GADD45B, B4GALT5, IL15RA, TNFAIP3, and SOCS1. In some embodiments, the one or more genes set forth in Table 1 comprises all 11 of CD38, SOCS3, AQP9, CDKN1A, GADD45B, B4GALT5, IL15RA, TNFAIP3, SOCS1, IL1RN, and PFKFB3. In some embodiments, the one or more genes set forth in Table 1 comprises all 12 of CD38, SOCS3, AQP9, CDKN1A, GADD45B, B4GALT5, IL15RA, TNFAIP3, SOCS1, IL1RN, PFKFB3, and BCL2A1. In some embodiments, the one or more genes set forth in Table 1 are all 24 genes set forth in Table 1. In some embodiments, the expression level of the one or more genes set forth in Table 1 is increased in the sample obtained from the patient relative to the reference expression level. In some embodiments, the expression level of the one or more genes set forth in Table 1 is increased at least about 0.5-fold relative to the reference expression level. In some embodiments, the expression level of the one or more genes set forth in Table 1 is increased at least about 1-fold relative to the reference expression level. In some embodiments, the expression level of the one or more genes set forth in Table 1 is increased at least about 2-fold relative to the reference expression level. In some embodiments, the expression level of the one or more genes set forth in Table 1 is increased at least about 3-fold relative to the reference expression level. In some embodiments, the expression level of the one or more genes set forth in Table 1 is increased at least about 4-fold relative to the reference expression level. In some embodiments, the expression level of the one or more genes set forth in Table 1 is increased at least about 5-fold relative to the reference expression level. In some embodiments, the expression level of the one or more genes set forth in Table 1 is increased at least about 10-fold relative to the reference expression level. In some embodiments, the patient has an increased expression level of the one or more genes set forth in Table 1 relative to the reference expression level and the method further comprises administering to the patient an IRAK4 pathway inhibitor.

In fifth aspect, the invention features a method of treating a patient having an IRAK4-mediated disorder or condition, the method comprising administering to the patient an IRAK4 pathway inhibitor, wherein prior to treatment the expression level of one or more genes set forth in Table 1 (i.e., CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and ACSL1) in a sample obtained from the patient has been determined to be increased relative to a reference expression level. In some embodiments, the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, PFKFB3, and BCL2A1. In some embodiments, the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, and PFKFB3. In some embodiments, the one or more genes set forth in Table 1 comprises one or more genes selected from the group consisting of CD38, SOCS3, AQP9, CDKN1A, GADD45B, B4GALT5, IL15RA, TNFAIP3, and SOCS1. In some embodiments, the one or more genes set forth in Table 1 comprises all nine of CD38, SOCS3, AQP9, CDKN1A, GADD45B, B4GALT5, IL15RA, TNFAIP3, and SOCS1. In some embodiments, the one or more genes set forth in Table 1 comprises all 11 of CD38, SOCS3, AQP9, CDKN1A, GADD45B, B4GALT5, IL15RA, TNFAIP3, SOCS1, IL1RN, and PFKFB3. In some embodiments, the one or more genes set forth in Table 1 comprises all 12 of CD38, SOCS3, AQP9, CDKN1A, GADD45B, B4GALT5, IL15RA, TNFAIP3, SOCS1, IL1RN, PFKFB3, and BCL2A1. In some embodiments, the one or more genes set forth in Table 1 are all 24 genes set forth in Table 1. In some embodiments, the expression level of the one or more genes set forth in Table 1 have been determined to be increased at least about 0.5-fold relative to the reference expression level. In some embodiments, the expression level of the one or more genes set forth in Table 1 have been determined to be increased at least about 1-fold relative to the reference expression level. In some embodiments, the expression level of the one or more genes set forth in Table 1 have been determined to be increased at least about 2-fold relative to the reference expression level. In some embodiments, the expression level of the one or more genes set forth in Table 1 have been determined to be increased at least about 3-fold relative to the reference expression level. In some embodiments, the expression level of the one or more genes set forth in Table 1 have been determined to be increased at least about 4-fold relative to the reference expression level.

In some embodiments, the expression level of the one or more genes set forth in Table 1 have been determined to be increased at least about 5-fold relative to the reference expression level. In some embodiments, the expression level of the one or more genes set forth in Table 1 have been determined to be increased at least about 10-fold relative to the reference expression level.

In some embodiments of any one of the third, fourth, and fifth aspects, the reference expression level is: (i) the expression level of the one or more genes set forth in Table 1 in a reference population; or (ii) a pre-assigned expression level for the one or more genes set forth in Table 1.

In some embodiments of any one of the preceding aspects, the expression level of the one or more genes set forth in Table 1 in a reference population is a median expression level of the one or more genes set forth in Table 1 in a reference population.

In some embodiments of any one of the preceding aspects, the sample obtained from the patient is a tissue sample, a whole blood sample, a plasma sample, or a serum sample. In some embodiments, the sample obtained from the patient is a blood sample (e.g., a whole blood sample).

In some embodiments of any one of the preceding aspects, the expression level is an mRNA expression level. In some embodiments, the mRNA expression level is determined by RNA-Seq, qPCR, microarray analysis, gene expression profiling, serial analysis of gene expression, or whole genome sequencing. In some embodiments, the mRNA expression level is determined by qPCR. In other embodiments of any one of the preceding aspects, the expression level is a protein expression level.

In some embodiments of any one of the preceding aspects, the IRAK4-mediated disorder or condition is selected from the group consisting of an immune disorder, an inflammatory disorder, a fibrotic disorder, an eosinophilic disorder, an infection, pain, a central nervous system disorder, an acute kidney injury, a chronic kidney disease, endometriosis, non-alcoholic fatty liver disease, a metabolic syndrome, and obesity. In some embodiments, the immune disorder is lupus, asthma, atopic dermatitis, rheumatoid arthritis, inflammatory bowel disease (IBD), Crohn's disease, or ulcerative colitis. In some embodiments, the inflammatory disorder is lupus, asthma, atopic dermatitis, rheumatoid arthritis, inflammatory bowel disease (IBD), Crohn's disease, or ulcerative colitis. In some embodiments, the lupus is systemic lupus erythematosus (SLE). In some embodiments, the lupus is lupus nephritis.

In some embodiments of any one of the preceding aspects, the IRAK4 pathway inhibitor is an IRAK4 inhibitor, an IRAK1 inhibitor, a toll-like receptor (TLR) inhibitor, an interleukin-1 receptor (IL-1R) inhibitor, an interleukin-33 receptor (IL-33R) inhibitor, or a myeloid differentiation primary response gene 88 (MyD88) inhibitor. In some embodiments, the IRAK4 pathway inhibitor is an IRAK4 inhibitor. In some embodiments, the IRAK4 pathway inhibitor is a TLR inhibitor. In some embodiments, the TLR inhibitor is a TLR7 inhibitor, a TLR8 inhibitor, a TLR9 inhibitor, a TLR1 inhibitor, a TLR2 inhibitor, a TLR4 inhibitor, a TLRS inhibitor, a TLR6 inhibitor, or a TLR10 inhibitor. In some embodiments, the TLR inhibitor is a TLR7 inhibitor, a TLR8 inhibitor, or both a TLR7 and TLR8 inhibitor. In some embodiments, the TLR inhibitor is a TLR9 inhibitor. In some embodiments, the IRAK4 pathway inhibitor is a small molecule inhibitor.

In some embodiments of any one of the preceding aspects, the method further comprises administering to the patient an additional therapeutic agent. In some embodiments, the additional therapeutic agent is a corticosteroid, a nonsteroidal anti-inflammatory drug (NSAID), chloroquine, hydroxychloroquine (PLAQUENIL®), cyclosporine, azathioprine, methotrexate, mycophenolate mofetil (CELLCEPT®), or cyclophosphamide (CYTOXAN®). In some embodiments, the IRAK4 pathway inhibitor and the additional therapeutic agent are co-administered. In some embodiments, the IRAK4 pathway inhibitor and the additional therapeutic agent are sequentially administered.

In another aspect, the invention features a kit for identifying a patient having an IRAK4-mediated disorder or condition who may benefit from treatment comprising an IRAK4 pathway inhibitor, the kit comprising: (a) polypeptides or polynucleotides capable of determining the expression level of one or more genes set forth in Table 1 (i.e., CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and ACSL1); and (b) instructions for using the polypeptides or polynucleotides to identify a patient having an IRAK4-mediated disorder or condition who may benefit from treatment comprising the IRAK4 pathway inhibitor. In some embodiments, the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, PFKFB3, and BCL2A1. In some embodiments, the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, and PFKFB3. In some embodiments, the one or more genes set forth in Table 1 comprises one or more genes selected from the group consisting of CD38, SOCS3, AQP9, CDKN1A, GADD45B, B4GALT5, IL15RA, TNFAIP3, and SOCS1. In some embodiments, the one or more genes set forth in Table 1 comprises all nine of CD38, SOCS3, AQP9, CDKN1A, GADD45B, B4GALT5, IL15RA, TNFAIP3, and SOCS1. In some embodiments, the one or more genes set forth in Table 1 comprises all 11 of CD38, SOCS3, AQP9, CDKN1A, GADD45B, B4GALT5, IL15RA, TNFAIP3, SOCS1, IL1RN, and PFKFB3. In some embodiments, the one or more genes set forth in Table 1 comprises all 12 of CD38, SOCS3, AQP9, CDKN1A, GADD45B, B4GALT5, IL15RA, TNFAIP3, SOCS1, IL1RN, PFKFB3, and BCL2A1. In some embodiments, the one or more genes set forth in Table 1 are all 24 genes set forth in Table 1.

### BRIEF DESCRIPTION OF THE DRAWINGS

The application file contains at least one drawing executed in color. Copies of this patent or patent application with color drawings will be provided by the Office upon request and payment of the necessary fee.

FIG. 1A is a heatmap showing that 285 genes from a microarray dataset of GEO Accession GSE25742 (Alsina et al. Nat. lmmunol. 15:1134-42, 2014) showed significantly lower induction by the TLR7/8 stimulator R848 (resiquimod) in the whole blood from IRAK4-deficient patients compared to healthy patient controls (false discovery rate (FDR) <0.05; fold-change (FC) >1.25).

FIG. 1B is a graph showing the relative signature scores for the 285 genes that displayed significantly lower induction by R848 (resiquimod) in both IRAK4^{-/-} and MyD88^{-/-} patients as compared to R848-treated healthy patient controls.

FIG. 2 is a heatmap showing that IRAK4^{-/-} patients failed to upregulate type I IFNs and other TLR-regulated genes in response to R848 compared to healthy patients.

FIG. 3 is a series of graphs showing three genes (IL1RN, CLEC4E, and SMSN1) out of 44 identified genes that are differentially upregulated in systemic lupus erythematosus (SLE) patients from two extra-renal cohorts (University of Michigan Cohort and ROSE Phase II Study Cohort) compared to healthy patients from the respective cohorts. p<0.05; FC>1.2.

FIG. 4 is a series of graphs showing that IRAK4 pathway biomarker genes (CXCL10 and CD38 shown) displayed significantly impaired induction by R848 in bone marrow-derived macrophages (BMDMs) from IRAK4 kinase-dead (KD) mice compared to IRAK4 wild-type mice.

FIG. 5 is a graph showing that IFNβ1 was induced by R848 to a significantly lower extend (p=0.02) in IRAK4 KD mice compared to IRAK4 wild-type mice macrophages.

FIG. 6 is a table showing all 24 identified IRAK4 pathway biomarker genes and their respective expression levels following induction by R848 in human IRAK4^{-/-} whole blood (left column) and IRAK4 KD mice macrophages (middle column) compared to healthy and wild-type controls, respectively. The right column shows the relative expression levels for each IRAK4 biomarker in SLE patients relative to healthy patient controls.

FIG. 7A is a graph showing that the expression level of the IFN-regulated gene OAS1A trends towards decreased induction by R848 in IRAK4 KD mice compared to IRAK4 wild-type mice (p<0.15).

FIG. 7B is a graph showing that the expression level of the IFN-regulated gene OAS2 trends towards decreased induction by R848 in IRAK4 KD mice compared to IRAK4 wild-type mice (p<0.15).

FIG. 7C is a graph showing that the expression level of the IFN-regulated gene IFIT1 trends towards decreased induction by R848 in IRAK4 KD mice compared to IRAK4 wild-type mice (p<0.15).

FIG. 7D is a graph showing that the expression level of the IFN-regulated gene IFNA5 trends towards decreased induction by R848 in IRAK4 KD mice compared to IRAK4 wild-type mice (p<0.15). FIG. 7E is a graph showing that the expression level of the IFN-regulated gene MX1 trends towards decreased induction by R848 in IRAK4 KD mice compared to IRAK4 wild-type mice (p<0.15).

FIG. 8 is a table showing the results of dose-escalation experiments using two distinct IRAK4 small molecule inhibitors G03074387 (G-4387) (BMS) and G03081557 (G-1557) (Pfizer) in human whole blood samples, with or without stimulation by R848. IRAK4 biomarker genes that displayed a dose-dependent downregulation by the test IRAK4 small molecule inhibitor in at least two out of three of the tested human donor samples is identified by a “Y.”

FIG. 9 is a series of graphs showing the percent decrease in expression of the top nine IRAK4 biomarker genes, which displayed dose-dependent downregulation by both IRAK4 small molecule inhibitors G-4387 and G-1557. Respective p-values are also shown.

FIG. 10A is a heatmap showing the correlation coefficients for the denoted 12 IRAK4 biomarker genes, as determined from SLE patient blood samples (whole blood samples) from the ROSE Phase II Study Cohort described herein.

FIG. 10B is a heatmap showing the correlation coefficients for the denoted 12 IRAK4 biomarker genes, as determined from SLE patient blood samples (PBMC samples) from the University of Michigan Cohort described herein.

### DETAILED DESCRIPTION OF THE INVENTION

### I. Introduction

The present invention provides diagnostic methods, therapeutic methods, and compositions for the treatment of interleukin-1 receptor-associated kinase 4 (IRAK4)-mediated disorders or conditions (e.g., immune disorders (e.g., systemic lupus erythematosus) or inflammatory disorders (e.g., asthma). The invention is based, at least in part, on the discovery that expression levels of particular IRAK4 pathway genes (e.g., CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and ACSL1) can be used as biomarkers (e.g., prognostic biomarkers and/or predictive biomarkers) in diagnostic methods of monitoring the response of a patient having an IRAK4-mediated disorder or condition to treatment including an IRAK4 pathway inhibitor, methods of identifying a patient having an IRAK4-mediated disorder who may benefit from treatment including an IRAK4 pathway inhibitor, and methods of selecting a therapy for a patient having an IRAK4-mediated disorder or condition based on the expression level of one or more IRAK4 pathway genes. Also provided are related therapeutic methods and diagnostic kits.

### II. Definitions

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.”

The term “IRAK4 pathway inhibitor” refers to a molecule that decreases, blocks, inhibits, abrogates, or interferes with signal transduction through a pathway within which IRAK4 functions. In some embodiments, an IRAK4 pathway inhibitor may inhibit the activity of one or more proteins involved in the activation of IRAK4 signaling. In some embodiments, an IRAK4 signaling inhibitor may activate the activity of one or more proteins involved in the inhibition of IRAK4 signaling. IRAK4 pathway inhibitors include, but are not limited to, an IRAK4 inhibitor, an IRAK1 inhibitor, a toll-like receptor (TLR) inhibitor, an interleukin-1 receptor (IL-1R) inhibitor, an interleukin-33 receptor (IL-33R) inhibitor, or a myeloid differentiation primary response gene 88 (MyD88) inhibitor.

The term “IRAK4 inhibitor” or “IRAK4 antagonist” refers to molecule that decreases, blocks, inhibits, abrogates, or interferes with IRAK4 activation or function. In a particular embodiment, an IRAK4 inhibitor has a binding affinity (dissociation constant) to IRAK4 of about 1,000 nM or less. In another embodiment, an IRAK4 inhibitor has a binding affinity to IRAK4 of about 100 nM or less. In another embodiment, an IRAK4 inhibitor has a binding affinity to IRAK4 of about 50 nM or less. In another embodiment, an IRAK4 inhibitor has a binding affinity to IRAK4 of about 10 nM or less. In another embodiment, an IRAK4 inhibitor has a binding affinity to IRAK4 of about 1 nM or less. In a particular embodiment, an IRAK4 inhibitor inhibits IRAK4 signaling with an IC50 of 1,000 nM or less. In another embodiment, an IRAK4 inhibitor inhibits IRAK4 signaling with an IC50 of 500 nM or less. In another embodiment, an IRAK4 inhibitor inhibits IRAK4 signaling with an IC50 of 50 nM or less. In another embodiment, an IRAK4 inhibitor inhibits IRAK4 signaling with an IC50 of 10 nM or less. In another embodiment, an IRAK4 inhibitor inhibits IRAK4 signaling with an IC50 of 1 nM or less. In some embodiments, the IRAK4 inhibitor is a small molecule inhibitor of IRAK4.

The term “IRAK1 inhibitor” or “IRAK1 antagonist” refers to molecule that decreases, blocks, inhibits, abrogates, or interferes with IRAK1 activation or function. In a particular embodiment, an IRAK1 inhibitor has a binding affinity (dissociation constant) to IRAK1 of about 1,000 nM or less. In another embodiment, an IRAK1 inhibitor has a binding affinity to IRAK1 of about 100 nM or less. In another embodiment, an IRAK1 inhibitor has a binding affinity to IRAK1 of about 50 nM or less. In another embodiment, an IRAK1 inhibitor has a binding affinity to IRAK1 of about 10 nM or less. In another embodiment, an IRAK1 inhibitor has a binding affinity to IRAK1 of about 1 nM or less. In a particular embodiment, an IRAK1 inhibitor inhibits IRAK1 signaling with an IC50 of 1,000 nM or less. In another embodiment, an IRAK1 inhibitor inhibits IRAK1 signaling with an IC50 of 500 nM or less. In another embodiment, an IRAK1 inhibitor inhibits IRAK1 signaling with an IC50 of 50 nM or less. In another embodiment, an IRAK1 inhibitor inhibits IRAK1 signaling with an IC50 of 10 nM or less. In another embodiment, an IRAK1 inhibitor inhibits IRAK1 signaling with an IC50 of 1 nM or less. In some embodiments, the IRAK1 inhibitor is a small molecule inhibitor of IRAK1.

The term “toll-like receptor inhibitor,” “toll-like receptor antagonist,” “TLR inhibitor,” or “TLR antagonist” refers to molecule that decreases, blocks, inhibits, abrogates, or interferes with TLR (e.g., TLR7, TLR8, TLR9, TLR1, TLR2, TLR4, TLRS, TLR6, and/or TLR10) activation or function. In a particular embodiment, a TLR inhibitor has a binding affinity (dissociation constant) to TLR of about 1,000 nM or less. In another embodiment, a TLR inhibitor has a binding affinity to TLR of about 100 nM or less. In another embodiment, a TLR inhibitor has a binding affinity to TLR of about 50 nM or less. In another embodiment, a TLR inhibitor has a binding affinity to TLR of about 10 nM or less. In another embodiment, a TLR inhibitor has a binding affinity to TLR of about 1 nM or less. In a particular embodiment, a TLR inhibitor inhibits TLR signaling with an IC50 of 1,000 nM or less. In another embodiment, a TLR inhibitor inhibits TLR signaling with an IC50 of 500 nM or less. In another embodiment, a TLR inhibitor inhibits TLR signaling with an IC50 of 50 nM or less. In another embodiment, a TLR inhibitor inhibits TLR signaling with an IC50 of 10 nM or less. In another embodiment, a TLR inhibitor inhibits TLR signaling with an IC50 of 1 nM or less. In some embodiments, the TLR inhibitor is a small molecule inhibitor of one or more TLRs.

The term “interleukin-1 receptor inhibitor,” “interleukin-1 receptor antagonist,” “IL-1R inhibitor,” or “IL-1R antagonist” refers to molecule that decreases, blocks, inhibits, abrogates, or interferes with IL-1R activation or function. In a particular embodiment, an IL-1R inhibitor has a binding affinity (dissociation constant) to IL-1R of about 1,000 nM or less. In another embodiment, an IL-1R inhibitor has a binding affinity to IL-1R of about 100 nM or less. In another embodiment, an IL-1R inhibitor has a binding affinity to IL-1R of about 50 nM or less. In another embodiment, an IL-1 R inhibitor has a binding affinity to IL-1R of about 10 nM or less. In another embodiment, an IL-1R inhibitor has a binding affinity to IL-1R of about 1 nM or less. In a particular embodiment, an IL-1R inhibitor inhibits IL-1R signaling with an IC50 of 1,000 nM or less. In another embodiment, an IL-1R inhibitor inhibits IL-1R signaling with an IC50 of 500 nM or less. In another embodiment, an IL-1R inhibitor inhibits IL-1R signaling with an IC50 of 50 nM or less. In another embodiment, an IL-1R inhibitor inhibits IL-1R signaling with an IC50 of 10 nM or less. In another embodiment, an IL-1R inhibitor inhibits IL-1R signaling with an IC50 of 1 nM or less. In some embodiments, the IL-1R inhibitor is a small molecule inhibitor of IL-1R.

The term “interleukin-33 receptor inhibitor,” “interleukin-33 receptor antagonist,” “IL-33R inhibitor,” or “IL-33R antagonist” refers to molecule that decreases, blocks, inhibits, abrogates, or interferes with IL-33R activation or function. In a particular embodiment, an IL-33R inhibitor has a binding affinity (dissociation constant) to IL-33R of about 1,000 nM or less. In another embodiment, an IL-33R inhibitor has a binding affinity to IL-33R of about 100 nM or less. In another embodiment, an IL-33R inhibitor has a binding affinity to

IL-33R of about 50 nM or less. In another embodiment, an IL-33R inhibitor has a binding affinity to IL-33R of about 10 nM or less. In another embodiment, an IL-33R inhibitor has a binding affinity to IL-33R of about 1 nM or less. In a particular embodiment, an IL-33R inhibitor inhibits IL-33R signaling with an IC50 of 1,000 nM or less. In another embodiment, an IL-33R inhibitor inhibits IL-33R signaling with an IC50 of 500 nM or less. In another embodiment, an IL-33R inhibitor inhibits IL-33R signaling with an IC50 of 50 nM or less. In another embodiment, an IL-33R inhibitor inhibits IL-33R signaling with an IC50 of 10 nM or less. In another embodiment, an IL-33R inhibitor inhibits IL-33R signaling with an IC50 of 1 nM or less. In some embodiments, the IL-33R inhibitor is a small molecule inhibitor of IL-33R.

The term “myeloid differentiation primary response gene 88 inhibitor,” “myeloid differentiation primary response gene 88 antagonist,” “MyD88 inhibitor,” or “MyD88 antagonist” refers to molecule that decreases, blocks, inhibits, abrogates, or interferes with MyD88 activation or function. In a particular embodiment, a MyD88 inhibitor has a binding affinity (dissociation constant) to MyD88 of about 1,000 nM or less. In another embodiment, a MyD88 inhibitor has a binding affinity to MyD88 of about 100 nM or less. In another embodiment, a MyD88 inhibitor has a binding affinity to MyD88 of about 50 nM or less. In another embodiment, a MyD88 inhibitor has a binding affinity to MyD88 of about 10 nM or less. In another embodiment, a MyD88 inhibitor has a binding affinity to MyD88 of about 1 nM or less. In a particular embodiment, a MyD88 inhibitor inhibits MyD88 signaling with an IC50 of 1,000 nM or less. In another embodiment, a MyD88 inhibitor inhibits MyD88 signaling with an IC50 of 500 nM or less. In another embodiment, a MyD88 inhibitor inhibits MyD88 signaling with an IC50 of 50 nM or less. In another embodiment, a MyD88 inhibitor inhibits MyD88 signaling with an IC50 of 10 nM or less. In another embodiment, a MyD88 inhibitor inhibits MyD88 signaling with an IC50 of 1 nM or less. In some embodiments, the MyD88 inhibitor is a small molecule inhibitor of MyD88.

The term “CD38” refers to cluster of differentiation 38 and encompasses homologues, mutations, and isoforms thereof. CD38 is also referred to in the art as ADPRC1. The term encompasses full-length, unprocessed CD38, as well as any form of CD38 that results from processing in the cell. The term encompasses naturally occurring variants of CD38 (e.g., splice variants or allelic variants). The term encompasses, for example, the CD38 gene, the mRNA sequence of human CD38 (e.g., SEQ ID NO: 1; GenBank Accession No. NM_001775.3), and the amino acid sequence of human CD38 (e.g., SEQ ID NO: 2; UniProtKB Accession No. P28907) as well as CD38 DNA, mRNA, and amino acid sequences from any other vertebrate source, including mammals such as primates and rodents (e.g., mice and rats).

The term “SOCS3” refers to Suppressor Of Cytokine Signaling 3 and encompasses homologues, mutations, and isoforms thereof. SOCS3 is also referred to in the art as Cytokine-Inducible SH2 Protein 3 (CIS3), STAT-Induced STAT Inhibitor 3 (SS13), and ATOD4. The term encompasses full-length, unprocessed SOCS3, as well as any form of SOCS3 that results from processing in the cell. The term encompasses naturally occurring variants of SOCS3 (e.g., splice variants or allelic variants). The term encompasses, for example, the SOCS3 gene, the mRNA sequence of human SOCS3 (e.g., SEQ ID NO: 3; GenBank Accession No. NM_003955.4), and the amino acid sequence of human SOCS3 (e.g., SEQ ID NO: 4; UniProtKB Accession No. O14543) as well as SOCS3 DNA, mRNA, and amino acid sequences from any other vertebrate source, including mammals such as primates and rodents (e.g., mice and rats).

The term “AQP9” refers to aquaporin 9 and encompasses homologues, mutations, and isoforms thereof. AQP9 is also referred to in the art as Aquaglyceroporin-9, HsT17287, T17287, and Small Solute Channel 1 (SSC1). The term encompasses full-length, unprocessed AQP9, as well as any form of AQP9 that results from processing in the cell. The term encompasses naturally occurring variants of AQP9 (e.g., splice variants or allelic variants). The term encompasses, for example, the AQP9 gene, the mRNA sequence of human AQP9 (e.g., SEQ ID NO: 5; GenBank Accession No. NM_020980.4), and the amino acid sequence of human AQP9 (e.g., SEQ ID NO: 6; UniProtKB Accession No. O43315) as well as AQP9 DNA, mRNA, and amino acid sequences from any other vertebrate source, including mammals such as primates and rodents (e.g., mice and rats).

The term “CDKN1A” refers to Cyclin Dependent Kinase Inhibitor 1A and encompasses homologues, mutations, and isoforms thereof. CDKN1A is also referred to in the art as CDK-Interacting Protein 1 (CIP1), Melanoma Differentiation Associated Protein 6 (MDA-6), or Wild-Type P53-Activated Fragment 1 (WAF-1). The term encompasses full-length, unprocessed CDKN1A, as well as any form of CDKN1A that results from processing in the cell. The term encompasses naturally occurring variants of CDKN1A (e.g., splice variants or allelic variants). The term encompasses, for example, the CDKN1A gene, the mRNA sequence of human CDKN1A (e.g., SEQ ID NO: 7; GenBank Accession No. NM_000389), and the amino acid sequence of human CDKN1A (e.g., SEQ ID NO: 8; UniProtKB Accession No. P38936) as well as CDKN1A DNA, mRNA, and amino acid sequences from any other vertebrate source, including mammals such as primates and rodents (e.g., mice and rats).

The term “GADD45B” refers to Growth Arrest And DNA Damage Inducible Beta and encompasses homologues, mutations, and isoforms thereof. GADD45B is also referred to in the art as Myeloid Differentiation Primary Response Protein MyD118 (MYD118). The term encompasses full-length, unprocessed GADD45B, as well as any form of GADD45B that results from processing in the cell. The term encompasses naturally occurring variants of GADD45B (e.g., splice variants or allelic variants). The term encompasses, for example, the GADD45B gene, the mRNA sequence of human GADD45B (e.g., SEQ ID NO: 9; GenBank Accession No. NM_015675), and the amino acid sequence of human GADD45B (e.g., SEQ ID NO: 10; UniProtKB Accession No. O75293) as well as GADD45B DNA, mRNA, and amino acid sequences from any other vertebrate source, including mammals such as primates and rodents (e.g., mice and rats).

The term “B4GALT5” refers to Beta-1,4-Galactosyltransferase 5 and encompasses homologues, mutations, and isoforms thereof. B4GALT5 is also referred to in the art as UDP-Galactose: Beta-N-Acetylglucosamine. The term encompasses full-length, unprocessed B4GALT5, as well as any form of B4GALT5 that results from processing in the cell. The term encompasses naturally occurring variants of B4GALT5 (e.g., splice variants or allelic variants). The term encompasses, for example, the B4GALT5 gene, the mRNA sequence of human B4GALT5 (e.g., SEQ ID NO: 11; GenBank Accession No. NM_004776), and the amino acid sequence of human B4GALT5 (e.g., SEQ ID NO: 12; UniProtKB Accession No. O43286) as well as B4GALT5 DNA, mRNA, and amino acid sequences from any other vertebrate source, including mammals such as primates and rodents (e.g., mice and rats).

The term “IL15RA” refers to Interleukin 15 Receptor Subunit Alpha and encompasses homologues, mutations, and isoforms thereof. IL15RA is also referred to in the art as CD215. The term encompasses full-length, unprocessed IL15RA, as well as any form of IL15RA that results from processing in the cell. The term encompasses naturally occurring variants of IL15RA (e.g., splice variants or allelic variants). The term encompasses, for example, the IL15RA gene, the mRNA sequence of human IL15RA (e.g., SEQ ID NO: 13; GenBank Accession No. NM_008358), and the amino acid sequence of human IL15RA (e.g., SEQ ID NO: 14; UniProtKB Accession No. Q13261) as well as IL15RA DNA, mRNA, and amino acid sequences from any other vertebrate source, including mammals such as primates and rodents (e.g., mice and rats).

The term “TNFAIP3” refers to TNF alpha induced protein 3 and encompasses homologues, mutations, and isoforms thereof. TNFAIP3 is also referred to in the art as A20, OTUD7C, or AISBL. The term encompasses full-length, unprocessed TNFAIP3, as well as any form of TNFAIP3 that results from processing in the cell. The term encompasses naturally occurring variants of TNFAIP3 (e.g., splice variants or allelic variants). The term encompasses, for example, the TNFAIP3 gene, the mRNA sequence of human TNFAIP3 (e.g., SEQ ID NO: 15; GenBank Accession No. NM_001270508), and the amino acid sequence of human TNFAIP3 (e.g., SEQ ID NO: 16; UniProtKB Accession No. P21580) as well as TNFAIP3 DNA, mRNA, and amino acid sequences from any other vertebrate source, including mammals such as primates and rodents (e.g., mice and rats).

The term “SOCS1” refers to Suppressor Of Cytokine Signaling 1 and encompasses homologues, mutations, and isoforms thereof. SOCS1 is also referred to in the art as STAT-Induced STAT Inhibitor 1 (SSI1), Tec-Interacting Protein 3 (TIP3), Cytokine-Inducible SH2 Protein 1 (CISH1), or JAK Binding Protein. The term encompasses full-length, unprocessed SOCS1, as well as any form of SOCS1 that results from processing in the cell. The term encompasses naturally occurring variants of SOCS1 (e.g., splice variants or allelic variants). The term encompasses, for example, the SOCS1 gene, the mRNA sequence of human SOCS1 (e.g., SEQ ID NO: 17; GenBank Accession No. NM_003745), and the amino acid sequence of human SOCS1 (e.g., SEQ ID NO: 18; UniProtKB Accession No. O15524) as well as SOCS1 DNA, mRNA, and amino acid sequences from any other vertebrate source, including mammals such as primates and rodents (e.g., mice and rats).

The term “IL1RN” refers to Interleukin 1 Receptor Antagonist and encompasses homologues, mutations, and isoforms thereof. IL1RN is also referred to in the art as Anakinra, IRAP, DIRA, or MVCD4. The term encompasses full-length, unprocessed IL1RN, as well as any form of IL1RN that results from processing in the cell. The term encompasses naturally occurring variants of IL1RN (e.g., splice variants or allelic variants). The term encompasses, for example, the IL1RN gene, the mRNA sequence of human IL1RN (e.g., SEQ ID NO: 19; GenBank Accession No. NM_173842), and the amino acid sequence of human IL1RN (e.g., SEQ ID NO: 20; UniProtKB Accession No. P18510) as well as IL1RN DNA, mRNA, and amino acid sequences from any other vertebrate source, including mammals such as primates and rodents (e.g., mice and rats).

The term “PFKFB3” refers to 6-Phosphofructo-2-Kinase/Fructose-2,6-Biphosphatase 3 and encompasses homologues, mutations, and isoforms thereof. PFKFB3 is also referred to in the art as IPFK2, PFK2, or iPFK-2. The term encompasses full-length, unprocessed PFKFB3, as well as any form of PFKFB3 that results from processing in the cell. The term encompasses naturally occurring variants of PFKFB3 (e.g., splice variants or allelic variants). The term encompasses, for example, the PFKFB3 gene, the mRNA sequence of human PFKFB3 (e.g., SEQ ID NO: 21; GenBank Accession No. NM_004566), and the amino acid sequence of human PFKFB3 (e.g., SEQ ID NO: 22; UniProtKB Accession No. Q16875) as well as PFKFB3 DNA, mRNA, and amino acid sequences from any other vertebrate source, including mammals such as primates and rodents (e.g., mice and rats).

The term “BCL2A1” refers to BCL2 Related Protein Al and encompasses homologues, mutations, and isoforms thereof. BCL2A1 is also referred to in the art as GRS, ACC1, ACC2, BFL1, ACC-1, ACC-2, HBPA1, or BCL2L5. The term encompasses full-length, unprocessed BCL2A1, as well as any form of BCL2A1 that results from processing in the cell. The term encompasses naturally occurring variants of BCL2A1 (e.g., splice variants or allelic variants). The term encompasses, for example, the BCL2A1 gene, the mRNA sequence of human BCL2A1 (e.g., SEQ ID NO: 23; GenBank Accession No. NM_004049), and the amino acid sequence of human BCL2A1 (e.g., SEQ ID NO: 24; UniProtKB Accession No. Q16548) as well as BCL2A1 DNA, mRNA, and amino acid sequences from any other vertebrate source, including mammals such as primates and rodents (e.g., mice and rats).

The term “CXCL10” refers to C-X-C Motif Chemokine Ligand 10 and encompasses homologues, mutations, and isoforms thereof. CXCL10 is also referred to in the art as C7, IFI10, INP10, IP-10, crg-2, mob-1, SCYB10, or gIP-10. The term encompasses full-length, unprocessed CXCL10, as well as any form of CXCL10 that results from processing in the cell. The term encompasses naturally occurring variants of CXCL10 (e.g., splice variants or allelic variants). The term encompasses, for example, the CXCL10 gene, the mRNA sequence of human CXCL10 (e.g., SEQ ID NO: 25; GenBank Accession No. NM_001565), and the amino acid sequence of human CXCL10 (e.g., SEQ ID NO: 26; UniProtKB Accession No. P02778) as well as CXCL10 DNA, mRNA, and amino acid sequences from any other vertebrate source, including mammals such as primates and rodents (e.g., mice and rats).

The term “CCL8” refers to C-C Motif Chemokine Ligand 8 and encompasses homologues, mutations, and isoforms thereof. CCL8 is also referred to in the art as HC14, MCP2, MCP-2, SCYA8, or SCYA10. The term encompasses full-length, unprocessed CCL8, as well as any form of CCL8 that results from processing in the cell. The term encompasses naturally occurring variants of CCL8 (e.g., splice variants or allelic variants). The term encompasses, for example, the CCL8 gene, the mRNA sequence of human CCL8 (e.g., SEQ ID NO: 27; GenBank Accession No. NM_005623), and the amino acid sequence of human CCL8 (e.g., SEQ ID NO: 28; UniProtKB Accession No. P80075) as well as CCL8 DNA, mRNA, and amino acid sequences from any other vertebrate source, including mammals such as primates and rodents (e.g., mice and rats).

The term “GPR84” refers to G Protein-Coupled Receptor 84 and encompasses homologues, mutations, and isoforms thereof. GPR84 is also referred to in the art as EX33 or GPCR4. The term encompasses full-length, unprocessed GPR84, as well as any form of GPR84 that results from processing in the cell. The term encompasses naturally occurring variants of GPR84 (e.g., splice variants or allelic variants). The term encompasses, for example, the GPR84 gene, the mRNA sequence of human GPR84 (e.g., SEQ ID NO: 29; GenBank Accession No. NM_020370), and the amino acid sequence of human GPR84 (e.g., SEQ ID NO: 30; UniProtKB Accession No. Q9NQS5) as well as GPR84 DNA, mRNA, and amino acid sequences from any other vertebrate source, including mammals such as primates and rodents (e.g., mice and rats).

The term “C15orf48” refers to Chromosome 15 Open Reading Frame 48 and encompasses homologues, mutations, and isoforms thereof. C15orf48 is also referred to in the art as NMES1 or FOAP-11. The term encompasses full-length, unprocessed C15orf48, as well as any form of C15orf48 that results from processing in the cell. The term encompasses naturally occurring variants of C15orf48 (e.g., splice variants or allelic variants). The term encompasses, for example, the C15orf48 gene, the mRNA sequence of human C15orf48 (e.g., SEQ ID NO: 31; GenBank Accession No. NM_197955), and the amino acid sequence of human C15orf48 (e.g., SEQ ID NO: 32; UniProtKB Accession No. Q9C002) as well as C15orf48 DNA, mRNA, and amino acid sequences from any other vertebrate source, including mammals such as primates and rodents (e.g., mice and rats).

The term “DRAM1” refers to DNA Damage Regulated Autophagy Modulator 1 and encompasses homologues, mutations, and isoforms thereof. DRAM1 is also referred to in the art as DRAM. The term encompasses full-length, unprocessed DRAM1, as well as any form of DRAM1 that results from processing in the cell. The term encompasses naturally occurring variants of DRAM1 (e.g., splice variants or allelic variants). The term encompasses, for example, the DRAM1 gene, the mRNA sequence of human DRAM1 (e.g., SEQ ID NO: 33; GenBank Accession No. NM_018370), and the amino acid sequence of human DRAM1 (e.g., SEQ ID NO: 34; UniProtKB Accession No. Q8N682) as well as DRAM1 DNA, mRNA, and amino acid sequences from any other vertebrate source, including mammals such as primates and rodents (e.g., mice and rats).

The term “CXCL11” refers to C-X-C Motif Chemokine Ligand 11 and encompasses homologues, mutations, and isoforms thereof. CXCL11 is also referred to in the art as IP9, H174, IP-9, b-R1, I-TAC, SCYB11, or SCYB9B. The term encompasses full-length, unprocessed CXCL11, as well as any form of CXCL11 that results from processing in the cell. The term encompasses naturally occurring variants of CXCL11 (e.g., splice variants or allelic variants). The term encompasses, for example, the CXCL11 gene, the mRNA sequence of human CXCL11 (e.g., SEQ ID NO: 35; GenBank Accession No. NM_005409), and the amino acid sequence of human CXCL11 (e.g., SEQ ID NO: 36; UniProtKB Accession No. O14625) as well as CXCL11 DNA, mRNA, and amino acid sequences from any other vertebrate source, including mammals such as primates and rodents (e.g., mice and rats).

The term “TNFAIP6” refers to TNF Alpha Induced Protein 6 and encompasses homologues, mutations, and isoforms thereof. TNFAIP6 is also referred to in the art as TSG6 or TSG-6. The term encompasses full-length, unprocessed TNFAIP6, as well as any form of TNFAIP6 that results from processing in the cell. The term encompasses naturally occurring variants of TNFAIP6 (e.g., splice variants or allelic variants). The term encompasses, for example, the TNFAIP6 gene, the mRNA sequence of human TNFAIP6 (e.g., SEQ ID NO: 37; GenBank Accession No. NM_007115), and the amino acid sequence of human TNFAIP6 (e.g., SEQ ID NO: 38; UniProtKB Accession No. P98066) as well as TNFAIP6 DNA, mRNA, and amino acid sequences from any other vertebrate source, including mammals such as primates and rodents (e.g., mice and rats).

The term “CSRNP1” refers to Cysteine and Serine Rich Nuclear Protein 1 and encompasses homologues, mutations, and isoforms thereof. CSRNP1 is also referred to in the art as AXUD1, URAX1, TAIP-3, CSRNP-1, or FAM130B. The term encompasses full-length, unprocessed CSRNP1, as well as any form of CSRNP1 that results from processing in the cell. The term encompasses naturally occurring variants of CSRNP1 (e.g., splice variants or allelic variants). The term encompasses, for example, the CSRNP1 gene, the mRNA sequence of human CSRNP1 (e.g., SEQ ID NO: 39; GenBank Accession No. NM_033027), and the amino acid sequence of human CSRNP1 (e.g., SEQ ID NO: 40; UniProtKB Accession No. Q96S65) as well as CSRNP1 DNA, mRNA, and amino acid sequences from any other vertebrate source, including mammals such as primates and rodents (e.g., mice and rats).

The term “PLSCR1” refers to Phospholipid Scramblase 1 and encompasses homologues, mutations, and isoforms thereof. PLSCR1 is also referred to in the art as MMTRA1B. The term encompasses full-length, unprocessed PLSCR1, as well as any form of PLSCR1 that results from processing in the cell. The term encompasses naturally occurring variants of PLSCR1 (e.g., splice variants or allelic variants). The term encompasses, for example, the PLSCR1 gene, the mRNA sequence of human PLSCR1 (e.g., SEQ ID NO: 41; GenBank Accession No. NM_021105), and the amino acid sequence of human PLSCR1 (e.g., SEQ ID NO: 42; UniProtKB Accession No. O15162) as well as PLSCR1 DNA, mRNA, and amino acid sequences from any other vertebrate source, including mammals such as primates and rodents (e.g., mice and rats).

The term “CLEC4E” refers to C-Type Lectin Domain Family 4 Member E and encompasses homologues, mutations, and isoforms thereof. CLEC4E is also referred to in the art as MINCLE or CLECSF9. The term encompasses full-length, unprocessed CLEC4E, as well as any form of CLEC4E that results from processing in the cell. The term encompasses naturally occurring variants of CLEC4E (e.g., splice variants or allelic variants). The term encompasses, for example, the CLEC4E gene, the mRNA sequence of human CLEC4E (e.g., SEQ ID NO: 43; GenBank Accession No. NM_014358), and the amino acid sequence of human CLEC4E (e.g., SEQ ID NO: 44; UniProtKB Accession No. Q9ULY5) as well as CLEC4E DNA, mRNA, and amino acid sequences from any other vertebrate source, including mammals such as primates and rodents (e.g., mice and rats).

The term “SAMSN1” refers to SAM Domain, SH3 Domain and Nuclear Localization Signals 1 and encompasses homologues, mutations, and isoforms thereof. SAMSN1 is also referred to in the art as SLy2, HACS1, NASH1, SASH2, or SH3D6B. The term encompasses full-length, unprocessed SAMSN1, as well as any form of SAMSN1 that results from processing in the cell. The term encompasses naturally occurring variants of SAMSN1 (e.g., splice variants or allelic variants). The term encompasses, for example, the SAMSN1 gene, the mRNA sequence of human SAMSN1 (e.g., SEQ ID NO: 45; GenBank Accession No. NM_022136), and the amino acid sequence of human SAMSN1 (e.g., SEQ ID NO: 46; UniProtKB Accession No. Q9NSI8) as well as SAMSN1 DNA, mRNA, and amino acid sequences from any other vertebrate source, including mammals such as primates and rodents (e.g., mice and rats).

The term “ACSL1” refers to Acyl-CoA Synthetase Long-Chain Family Member 1 and encompasses homologues, mutations, and isoforms thereof. ACSL1 is also referred to in the art as Acs, Acas, FACS, Acas1, Facl2, or LACS1. The term encompasses full-length, unprocessed ACSL1, as well as any form of ACSL1that results from processing in the cell. The term encompasses naturally occurring variants of ACSL1 (e.g., splice variants or allelic variants). The term encompasses, for example, the ACSL1gene, the mRNA sequence of human ACSL1 (e.g., SEQ ID NO: 47; GenBank Accession No. NM_001286708), and the amino acid sequence of human ACSL1 (e.g., SEQ ID NO: 48; UniProtKB Accession No. P33121) as well as ACSL1 DNA, mRNA, and amino acid sequences from any other vertebrate source, including mammals such as primates and rodents (e.g., mice and rats).

As used herein, the terms “patient,” “individual,” and “subject” are used interchangeably and refer to any single animal, more preferably a mammal (including such non-human animals as, for example, cats, dogs, horses, rabbits, zoo animals, cows, pigs, sheep, and non-human primates) for which treatment is desired. In particular embodiments, the patient herein is a human. The patient may be a patient having, suspected of having, or at risk of suffering from an IRAK4-mediated disorder or condition (e.g., an immune disorder, an inflammatory disorder, a fibrotic disorder, an eosinophilic disorder, an infection, pain, a central nervous system disorder, an acute kidney injury, a chronic kidney disease, endometriosis, non-alcoholic fatty liver disease, a metabolic syndrome, and obesity). The patient may have been previously treated with an IRAK4 pathway inhibitor, another drug, or not previously treated. The patient may be naïve to an additional drug(s) being used when the treatment is started, i.e., the patient may not have been previously treated with, for example, a therapy other than one including an IRAK4 pathway inhibitor at “baseline” (i.e., at a set point in time before the administration of a first dose of an IRAK4 pathway inhibitor in the treatment method herein, such as the day of screening the subject before treatment is commenced). Such a “naïve” patient or subject is generally considered a candidate for treatment with such additional drug(s).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

“Polynucleotide” or “nucleic acid,” as used interchangeably herein, refers to polymers of nucleotides of any length and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single-stranded polynucleotide that is capable of hybridizing to a nucleic acid and allowing polymerization of a complementary nucleic acid, generally by providing a free 3′-OH group. The term “small molecule” refers to any molecule with a molecular weight of about 2000 daltons or less, preferably of about 500 daltons or less.

The term “detection” includes any means of detecting, including direct and indirect detection. The term “biomarker” as used herein refers to an indicator molecule or set of molecules (e.g., predictive, diagnostic, and/or prognostic indicator), which can be detected in a sample and includes, for example, CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and ACSL1. The biomarker may be a predictive biomarker and serve as an indicator of the likelihood of sensitivity or benefit of a patient having a particular disorder or condition (e.g., an IRAK4-mediated disorder or condition) to treatment with an IRAK4 pathway inhibitor. Biomarkers include, but are not limited to, polynucleotides (e.g., DNA and/or RNA (e.g., mRNA)), polynucleotide copy number alterations (e.g., DNA copy numbers), polypeptides, polypeptide and polynucleotide modifications (e.g., post-translational modifications), carbohydrates, and/or glycolipid-based molecular markers. In some embodiments, a biomarker is a gene.

The “amount” or “level” of a biomarker, as used herein, is a detectable level in a biological sample. These can be measured by methods known to one skilled in the art and also disclosed herein.

The term “expression level” or “level of expression” generally refers to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression,” “increased expression level,” “increased levels,” “elevated expression,” “elevated expression levels,” or “elevated levels” refers to an increased expression or increased levels of a biomarker in an individual relative to a control, such as an individual or individuals who do not have the disorder or condition (e.g., an IRAK4-mediated disorder or condition) (e.g., healthy individuals), an internal control (e.g., a housekeeping biomarker), or a median expression level of the biomarker in samples from a group/population of patients.

“Decreased expression,” “decreased expression level,” “decreased levels,” “reduced expression,” “reduced expression levels,” or “reduced levels” refers to a decrease expression or decreased levels of a biomarker in an individual relative to a control, such as an individual or individuals who do not have a disorder or condition (e.g., an IRAK4-mediated disorder or condition) (e.g., healthy individuals), an internal control (e.g., a housekeeping biomarker), or a median expression level of the biomarker in samples from a group/population of patients. In some embodiments, reduced expression is little or no expression.

The term “housekeeping gene” refers herein to a gene or group of genes that encode proteins whose activities are essential for the maintenance of cell function and which are typically similarly present in all cell types.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology, (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Quantitative polymerase chain reaction” or “qPCR” refers to a form of PCR wherein the amount of PCR product is measured at each step in a PCR reaction. This technique has been described in various publications including, for example, Cronin et al., Am. J. Pathol. 164(1):35-42 (2004) and Ma et al., Cancer Cell 5:607-616 (2004).

The term “microarray” refers to an ordered arrangement of hybridizable array elements, preferably polynucleotide probes, on a substrate.

The term “sample,” as used herein, refers to a composition that is obtained or derived from a subject (e.g., individual of interest) that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a tumor sample is a tissue sample obtained from a tumor or other cancerous tissue.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference level, reference sample, reference cell, reference tissue, control sample, control cell, or control tissue is obtained from a healthy and/or non-diseased part of the body (e.g., tissue or cells) of the same subject or individual. For example, the reference level, reference sample, reference cell, reference tissue, control sample, control cell, or control tissue may be healthy and/or non-diseased cells or tissue adjacent to the diseased cells or tissue. In another embodiment, a reference sample is obtained from an untreated tissue and/or cell of the body of the same subject or individual. In yet another embodiment, a reference level, reference sample, reference cell, reference tissue, control sample, control cell, or control tissue is obtained from a healthy and/or non-diseased part of the body (e.g., tissues or cells) of an individual who is not the subject or individual. In even another embodiment, a reference level, reference sample, reference cell, reference tissue, control sample, control cell, or control tissue is obtained from an untreated tissue and/or cell of the body of an individual who is not the subject or individual.

For the purposes herein a “section” of a tissue sample is meant a single part or piece of a tissue sample, for example, a thin slice of tissue or cells cut from a tissue sample. It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of progression of IRAK4-mediated disorder or condition, including slowing down or complete arrest; (2) relief, to some extent, of one or more symptoms associated with the IRAK4-mediated disorder or condition; (6) increase or extension in the length of survival, including overall survival and progression-free survival; and/or (7) decreased mortality at a given point of time following treatment.

The term “survival” refers to the patient remaining alive, and includes overall survival as well as progression-free survival.

As used herein, “progression-free survival” or “PFS” refers to the length of time during and after treatment during which the disease being treated (e.g., an IRAK4-mediated disorder or condition (e.g., immune disorder (e.g., systemic lupus erythematosus (SLE)) or inflammatory disorder (e.g., asthma)) does not progress or get worse. Progression-free survival may include the amount of time individuals have experienced a complete response or a partial response, as well as the amount of time individuals have experienced stable disease.

As used herein, “overall survival” or “OS” refers to the percentage of subjects in a group who are likely to be alive after a particular duration of time (e.g., 6 months, 1 year, 2 years, 3 years, 4 years, 5 years, 10 years, 15 years, 20 years, or more than 20 years from the time of diagnosis or treatment).

An “effective response” of a patient or a patient's “responsiveness” to treatment with a medicament and similar wording refers to the clinical or therapeutic benefit imparted to a patient at risk for, or having, an

IRAK4-mediated disorder or condition, such as an immune disorder (e.g., systemic lupus erythematosus (SLE)) or inflammatory disorder (e.g., asthma)). In one embodiment, one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1 is used to identify a patient who is predicted to have an increased likelihood of being responsive to treatment with a medicament (e.g., treatment including an IRAK4 pathway inhibitor), relative to a patient who does not express the one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1. In one embodiment, one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1 is used to identify the patient who is predicted to have an increase likelihood of being responsive to treatment with a medicament (e.g., treatment including an IRAK4 pathway inhibitor), relative to a patient who does not express the one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1 at the same level.

A “disorder” is any condition that would benefit from treatment including, but not limited to, chronic and acute disorders or diseases, including those pathological conditions that predispose the mammal to the disorder in question.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical formulation, other than an active ingredient, which is nontoxic to a subject. A pharmaceutically acceptable excipient includes, but is not limited to, a buffer, carrier, stabilizer, or preservative.

The term “pharmaceutically acceptable salt” denotes salts which are not biologically or otherwise undesirable. Pharmaceutically acceptable salts include both acid and base addition salts. The phrase “pharmaceutically acceptable” indicates that the substance or composition must be compatible chemically and/or toxicologically, with the other ingredients comprising a formulation, and/or the mammal being treated therewith.

The term “pharmaceutically acceptable acid addition salt” denotes those pharmaceutically acceptable salts formed with inorganic acids, such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids, such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid “mesylate”, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts.

Salts derived from pharmaceutically acceptable organic nontoxic bases includes salts of primary, secondary, and tertiary amines, substituted amines, including naturally occurring substituted amines, cyclic amines, and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, and polyamine resins.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, IRAK4 pathway inhibitors (e.g., IRAK4 inhibitors, IRAK1 inhibitors, toll-like receptor (TLR) inhibitors, interleukin-1 receptor (IL-1R) inhibitors, interleukin-33 receptor (IL-33R) inhibitors, or myeloid differentiation primary response gene 88 (MyD88) inhibitors) are used to delay development of an IRAK4-mediated disorder or condition or to slow the progression of an IRAK4-mediated disorder or condition.

As used herein, “administering” is meant a method of giving a dosage of a compound (e.g., an IRAK4 pathway inhibitor, such as an IRAK4 inhibitor or antagonist) or a pharmaceutical composition (e.g., a pharmaceutical composition including an inhibitor or antagonist) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “co-administered” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 10%, 20%, 30%, 40%, 50%, 60%, 70%, 75%, 80%, 85%, 90%, 95%, or greater. Reduce or inhibit can refer, for example, to the level of activity and/or function of a protein in the IRAK4 pathway (e.g., the level of signal transduction through the IRAK4 pathway). Additionally, reduce or inhibit can refer, for example, to the symptoms of the disorder or condition being treated (e.g., an IRAK4-mediated disorder or condition).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., an IRAK4-mediated disorder or condition), or a probe for specifically detecting a biomarker (e.g., CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and/or ACSL1) described herein. In certain embodiments, the manufacture or kit is promoted, distributed, or sold as a unit for performing the methods described herein.

The phrase “based on” when used herein means that the information about one or more biomarkers is used to inform a diagnostic decision, a treatment decision, information provided on a package insert, or marketing/promotional guidance, etc.

### III. Methods

A. Diagnostic Methods Based on the Expression Level of IRAK4 Pathway Biomarkers

The present invention provides diagnostic methods in which the IRAK4 pathway biomarkers identified herein serve as pharmacodynamic biomarkers.

Accordingly, the present invention features diagnostic methods of monitoring the response of a patient having an interleukin-1 receptor-associated kinase 4 (IRAK4)-mediated disorder or condition to treatment including an IRAK4 pathway inhibitor, the method including (a) determining, in a sample obtained from the patient at a time point following administration of a first dose of the IRAK4 pathway inhibitor, the expression level of one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1 below, and (b) comparing the expression level of the one or more genes set forth in Table 1 in the sample with a reference expression level, thereby monitoring the response of the patient to treatment including the IRAK4 pathway inhibitor.

TABLE 1
IRAK4 Pathway Biomarker Genes
Genes
CD38
SOCS3
AQP9
CDKN1A
GADD45B
B4GALT5
IL15RA
TNFAIP3
SOCS1
IL1RN
PFKFB3
BCL2A1
CXCL10
CCL8
GPR84
C15orf48
DRAM1
CXCL11
TNFAIP6
CSRNP1
PLSCR1
CLEC4E
SAMSN1
ACSL1

Accordingly, one could determine the expression levels of any combination of 2, 3, 4, 5, 6, 7, 8, 9, 10, 11, 12, 13, 14 , 15, 16, 17, 18, 19, 20, 21, 22, or 23 genes selected from the genes set forth in Table 1 in a sample obtained from the patient at a time point following administration of a first dose of the IRAK4 pathway inhibitor and, subsequently, compare the expression levels of the combination of genes with a reference expression level (e.g., the median expression levels of the same combination of genes in a reference population of individuals), thereby monitoring the response of the patient to treatment including the IRAK4 pathway inhibitor.

In certain instances, the method includes determining, in a sample obtained from the patient at a time point following administration of a first dose of the IRAK4 pathway inhibitor, the expression level of one or more genes (e.g., one gene, two genes, three genes, four genes, five genes, six genes, seven genes, eight genes, nine genes, ten genes, eleven genes, or all twelve genes) selected from the group consisting of CD38, SOCS3, AQP9, CDKN1A, GADD45B, B4GALT5, IL15RA, TNFAIP3, SOCS1, IL1 RN, PFKFB3, and BCL2A1. For example, in some instances, the method includes determining, in a sample obtained from the patient at a time point following administration of a first dose of the IRAK4 pathway inhibitor, the expression level of one or more genes (e.g., one gene, two genes, three genes, four genes, five genes, six genes, seven genes, eight genes, nine genes, ten genes, or all eleven genes) selected from the group consisting of CD38, SOCS3, AQP9, CDKN1A, GADD45B, B4GALT5, IL15RA, TNFAIP3, SOCS1, IL1RN, and PFKFB3. In particular instances, the method includes determining, in a sample obtained from the patient at a time point following administration of a first dose of the IRAK4 pathway inhibitor, the expression level of one or more genes (e.g., one gene, two genes, three genes, four genes, five genes, six genes, seven genes, eight genes, or all nine genes) selected from the group consisting of CD38, SOCS3, AQP9, CDKN1A, GADD45B, B4GALT5, IL15RA, TNFAIP3, and SOCS1.

In some instances, the expression level of the one or more genes set forth in Table 1 is decreased in the sample obtained from the patient at a time point following administration of a first dose of the IRAK4 pathway inhibitor relative to the reference expression level. For example, in some instances, the expression level of the one or more genes set forth in Table 1 is decreased by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, or about 100%), e.g., from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%, relative to a reference expression level (e.g., the expression level of the one or more genes set forth in Table 1 in a sample from the patient obtained prior to administration of the first dose of the IRAK4 pathway inhibitor). In other instances, the expression level of the one or more genes set forth in Table 1 is decreased by about 0.5-fold, about 0.6-fold, about 0.7-fold, about 0.8-fold, about 0.9-fold, about 1-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, or about 10-fold or greater, e.g., from about 0.5-fold to about 0.7-fold, from about 0.7-fold to about 1-fold, from about 1-fold to about 1.5-fold, from about 1.5-fold to about 2-fold, from about 2-fold to about 3-fold, from about 3-fold to about 4-fold, from about 4-fold to about 5-fold, from about 5-fold to about 6-fold, from about 6-fold to about 7-fold, from about 7-fold to about 8-fold, or from about 9-fold to about 10-fold or greater, relative to the reference expression level (e.g., the expression level of the one or more genes set forth in Table 1 in a sample from the patient obtained prior to administration of the first dose of the IRAK4 pathway inhibitor).

In some embodiments, reduced or decreased expression refers to an overall reduction of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference level, reference sample, reference cell, reference tissue, control sample, control cell, or control tissue. In certain embodiments, reduced expression refers to the decrease in expression level (amount) of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level (amount) of the respective biomarker in a reference level, reference sample, reference cell, reference tissue, control sample, control cell, or control tissue.

In any one the preceding embodiments, the decreased expression level of the one or more genes set forth in Table 1 may indicate that the patient is responding to the IRAK4 pathway inhibitor. Accordingly, in some instances, the method further includes administering at least a second dose (e.g., one, two, three, four, five, six, seven, eight, nine, or ten or more additional doses) of an IRAK4 pathway inhibitor to a patient whose expression level of the one or more genes set forth in Table 1 is decreased relative to the reference expression level.

In any one the preceding embodiments, the reference expression level can be: (i) the expression level of the one or more genes set forth in Table 1 in a sample from the patient obtained prior to administration of the first dose of the IRAK4 pathway inhibitor; (ii) the expression level of the one or more genes set forth in Table 1 in a reference population (e.g., a median expression level of the one or more genes set forth in Table 1 in a reference population); (iii) a pre-assigned expression level for the one or more genes set forth in Table 1; (iv) the expression level of the one or more genes set forth in Table 1 in a sample obtained from the patient at a previous time point, wherein the previous time point is following administration of the first dose of the IRAK4 pathway inhibitor; or (v) the expression level of the one or more genes set forth in Table 1 in a sample obtained from the patient at a subsequent time point.

The present invention provides diagnostic methods in which the IRAK4 pathway biomarkers identified herein serve as predictive biomarkers.

Accordingly, the present invention features diagnostic methods of identifying a patient having an IRAK4-mediated disorder or condition who may benefit from treatment comprising an IRAK4 pathway inhibitor, the method including determining an expression level of one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1 in a sample obtained from the patient, wherein an increased expression level of the one or more genes set forth in Table 1 in the sample as compared to a reference expression level identifies the patient as one who may benefit from treatment including an IRAK4 pathway inhibitor.

The present invention also features diagnostic methods of selecting a therapy for a patient having an IRAK4-mediated disorder or condition, the method including determining an expression level of one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1 in a sample obtained from the patient, wherein an increased expression level of the one or more genes set forth in Table 1 in the sample as compared to a reference expression level identifies the patient as one who may benefit from treatment including an IRAK4 pathway inhibitor.

In certain instances, the methods of identifying a patient or selecting a therapy for a patient may include determining the expression level of one or more genes (e.g., one gene, two genes, three genes, four genes, five genes, six genes, seven genes, eight genes, nine genes, ten genes, eleven genes, or all twelve genes) selected from the group consisting of CD38, SOCS3, AQP9, CDKN1A, GADD45B, B4GALT5, IL15RA, TNFAIP3, SOCS1, IL1RN, PFKFB3, and BCL2A1. For example, in some instances, the method includes determining the expression level of one or more genes (e.g., one gene, two genes, three genes, four genes, five genes, six genes, seven genes, eight genes, nine genes, ten genes, or all eleven genes) selected from the group consisting of CD38, SOCS3, AQP9, CDKN1A, GADD45B, B4GALT5, IL15RA, TNFAIP3, SOCS1, IL1RN, and PFKFB3. In particular instances, the method includes determining the expression level of one or more genes (e.g., one gene, two genes, three genes, four genes, five genes, six genes, seven genes, eight genes, or all nine genes) selected from the group consisting of CD38, SOCS3, AQP9, CDKN1A, GADD45B, B4GALT5, IL15RA, TNFAIP3, and SOCS1.

In some instances, the expression level of one or more genes (e.g., 1, 2, 3, 4, 5, 6, 7, 8, 9, 10, 11, 12, 13, 14, 15, 16, 17, 18, 19, 20, 21, 22, 23, or all 24 genes) set forth in Table 1 is increased in the sample obtained from the patient relative to the reference expression level. For example, in some instances, the expression level of the one or more genes set forth in Table 1 is increased by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, or about 100%), e.g., from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%, relative to a reference expression level (e.g., the expression level of the one or more genes set forth in Table 1 in a reference population, e.g., a median expression level of the one or more genes set forth in Table 1 in a reference population). In other instances, the expression level of the one or more genes set forth in Table 1 is increased by about 0.5-fold, about 0.6-fold, about 0.7-fold, about 0.8-fold, about 0.9-fold, about 1-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, or about 10-fold or greater, e.g., from about 0.5-fold to about 0.7-fold, from about 0.7-fold to about 1-fold, from about 1-fold to about 1.5-fold, from about 1.5-fold to about 2-fold, from about 2-fold to about 3-fold, from about 3-fold to about 4-fold, from about 4-fold to about 5-fold, from about 5-fold to about 6-fold, from about 6-fold to about 7-fold, from about 7-fold to about 8-fold, or from about 9-fold to about 10-fold or greater, relative to the reference expression level (e.g., e.g., the expression level of the one or more genes set forth in Table 1 in a reference population, e.g., a median expression level of the one or more genes set forth in Table 1 in a reference population). In some embodiments, elevated or increased expression refers to an overall increase of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art-known methods such as those described herein, as compared to a reference level, reference sample, reference cell, reference tissue, control sample, control cell, or control tissue.

In certain embodiments, the elevated or increased expression refers to the increase in expression level (amount) of a biomarker in the sample, wherein the increase is at least about any of 1.5×, 1.75×, 2×, 3×, 4×, 5×, 6×, 7×, 8×, 9×, 10×, 25×, 50×, 75×, or 100× the expression level (amount) of the respective biomarker in a reference level, reference sample, reference cell, reference tissue, control sample, control cell, or control tissue. In some embodiments, elevated expression refers to an overall increase of greater than about 1.5-fold, about 1.75-fold, about 2-fold, about 2.25-fold, about 2.5-fold, about 2.75-fold, about 3.0-fold, or about 3.25-fold as compared to a reference sample, reference cell, reference tissue, control sample, control cell, control tissue, or internal control (e.g., housekeeping gene). In some instances, the patient has an increased expression level of the one or more genes set forth in Table 1 relative to the reference expression level and the method further comprises administering to the patient an IRAK4 pathway inhibitor.

In some embodiments, the reference expression level can be: (i) the expression level of the one or more genes set forth in Table 1 in a reference population (e.g., a median expression level of the one or more genes set forth in Table 1 in a reference population); or (ii) a pre-assigned expression level for the one or more genes set forth in Table 1.

The diagnostic methods described above provide for convenient, efficient, and potentially cost-effective means to obtain data and information useful in assessing appropriate or effective therapies for treating patients. For example, the sample obtained from the patient can be a tissue sample, a whole blood sample, a plasma sample, or a serum sample. A patient can the sample before and/or after treatment with an IRAK4 pathway inhibitor, and the sample can be examined by way of various in vitro assays to determine whether the patient will likely benefit from treatment including an IRAK4 pathway inhibitor, such as an IRAK4 inhibitor, an IRAK1 inhibitor, a toll-like receptor (TLR) inhibitor, an interleukin-1 receptor (IL-1R) inhibitor, an interleukin-33 receptor (IL-33R) inhibitor, or a myeloid differentiation primary response gene 88 (MyD88) inhibitor.

In another aspect, the invention also provides methods for monitoring the sensitivity of a patient to an IRAK4 pathway inhibitor. The methods may be conducted in a variety of assay formats, including assays detecting genetic or protein expression levels and biochemical assays detecting appropriate activity. Determination of expression or the presence of such biomarkers in patient samples is predictive of whether a patient is sensitive to the biological effects of an IRAK4 pathway inhibitor. A difference or change (i.e., a decrease) in the expression of one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1 in a sample obtained from the patient at a time point following administration of a first dose of the IRAK4 pathway inhibitor relative to a reference level (e.g., the expression level of the one or more genes set forth in Table 1 in a sample from the patient obtained prior to administration of the first dose of the IRAK4 pathway inhibitor, the median expression level of the one or more genes in a sample from a group/population of patients being tested for sensitivity to the IRAK4 pathway inhibitor, or the median expression level of the one or more genes in a sample from a group/population of patients having a particular IRAK4-mediated disorder or condition) correlates with treatment efficacy of such a patient with an IRAK4 pathway inhibitor.

In another aspect, the invention provides a method of optimizing therapeutic efficacy of therapy for a patient having a IRAK4-mediated disorder or condition, including detecting, one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1 in a sample from the patient obtained (i) before an IRAK4 pathway inhibitor has been administered to the patient, (ii) after an IRAK4 pathway signaling inhibitor has been administered to the patient, or (iii) before and after such treatment. An increased expression level of one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1 in a sample obtained before an IRAK4 pathway inhibitor has been administered to the patient indicates that the patient will likely benefit from treatment including an IRAK4 pathway inhibitor, and the therapy for the patient having the IRAK4-mediated disorder or condition may be accordingly adjusted to include an IRAK4 pathway inhibitor. In other instances, a decreased expression level, relative to a reference expression level, of the one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1 following administration of the IRAK4 pathway indicates that the patient is responding to treatment with the IRAK4 pathway inhibitor, and treatment may optionally be continued, adjusted, or stopped accordingly. The patient may be informed that they have an increased likelihood of responding to treatment including an IRAK4 pathway inhibitor and/or provided a recommendation that treatment include an IRAK4 pathway inhibitor.

In any of the methods described herein, one could determine the expression levels of any combination of 2, 3, 4, 5, 6, 7, 8, 9, 10, 11, 12, 13, 14, 15, 16, 17, 18, 19, 20, 21, 22, or 23 genes selected from the genes set forth in Table 1 in a sample obtained from the patient.

In some embodiments of the diagnostic methods described herein, the expression levels of a combination of two genes set forth in Table 1, such as any of the exemplary combinations shown in Table 2, may be determined.

TABLE 2
Exemplary Two-Gene Combinations of IRAK4 Biomarkers

CD38 and SOCS3
CD38 and AQP9
CD38 and CDKN1A
CD38 and GADD45B
CD38 and B4GALT5
CD38 and IL15RA
CD38 and TNFAIP3
CD38 and SOCS1
SOCS3 and AQP9
SOCS3 and CDKN1A
SOCS3 and GADD45B
SOCS3 and B4GALT5
SOCS3 and IL15RA
SOCS3 and TNFAIP3
SOCS3 and SOCS1
AQP9 and CDKN1A
AQP9 and GADD45B
AQP9 and B4GALT5
AQP9 and IL15RA
AQP9 and TNFAIP3
AQP9 and SOCS1
CDKN1A and GADD45B
CDKN1A and B4GALT5
CDKN1A and IL15RA
CDKN1A and TNFAIP3
CDKN1A and SOCS1
GADD45B and B4GALT5
GADD45B and IL15RA
GADD45B and TNFAIP3
GADD45B and SOCS1
B4GALT5 and IL15RA
B4GALT5 and TNFAIP3
B4GALT5 and SOCS1
IL15RA and TNFAIP3
IL15RA and SOCS1
TNFAIP3 and SOCS1

In some embodiments of the diagnostic methods described herein, the expression levels of a combination of three genes set forth in Table 1, such as any of the exemplary combinations shown in Table 3, may be determined.

TABLE 3
Exemplary Three-Gene Combinations of IRAK4 Biomarkers

CD38, SOCS3, and AQP9
CD38, SOCS3, and CDKN1A
CD38, SOCS3, and GADD45B
CD38, SOCS3, and B4GALT5
CD38, SOCS3, and IL15RA
CD38, SOCS3, and TNFAIP3
CD38, SOCS3, and SOCS1
CD38, AQP9, and CDKN1A
CD38, AQP9, and GADD45B
CD38, AQP9, and B4GALT5
CD38, AQP9, and IL15RA
CD38, AQP9, and TNFAIP3
CD38, AQP9, and SOCS1
CD38, CDKN1A, and GADD45B
CD38, CDKN1A, and B4GALT5
CD38, CDKN1A, and IL15RA
CD38, CDKN1A, and TNFAIP3
CD38, CDKN1A, and SOCS1
CD38, GADD45B, and B4GALT5
CD38, GADD45B, and IL15RA
CD38, GADD45B, and TNFAIP3
CD38, GADD45B, and SOCS1
CD38, B4GALT5, and IL15RA
CD38, B4GALT5, and TNFAIP3
CD38, B4GALT5, and SOCS1
CD38, IL15RA, and TNFAIP3
CD38, IL15RA, and SOCS1
CD38, TNFAIP3, and SOCS1
SOCS3, AQP9, and CDKN1A
SOCS3, AQP9, and GADD45B
SOCS3, AQP9, and B4GALT5
SOCS3, AQP9, and IL15RA
SOCS3, AQP9, and TNFAIP3
SOCS3, AQP9, and SOCS1
SOCS3, CDKN1A, and GADD45B
SOCS3, CDKN1A, and B4GALT5
SOCS3, CDKN1A, and IL15RA
SOCS3, CDKN1A, and TNFAIP3
SOCS3, CDKN1A, and SOCS1
SOCS3, GADD45B, and B4GALT5
SOCS3, GADD45B, and IL15RA
SOCS3, GADD45B, and TNFAIP3
SOCS3, GADD45B, and SOCS1
SOCS3, B4GALT5, and IL15RA
SOCS3, B4GALT5, and TNFAIP3
SOCS3, B4GALT5, and SOCS1
SOCS3, IL15RA, and TNFAIP3
SOCS3, IL15RA, and SOCS1
SOCS3, TNFAIP3, and SOCS1
AQP9, CDKN1A, and GADD45B
AQP9, CDKN1A, and B4GALT5
AQP9, CDKN1A, and IL15RA
AQP9, CDKN1A, and TNFAIP3
AQP9, CDKN1A, and SOCS1
AQP9, GADD45B, and B4GALT5
AQP9, GADD45B, and IL15RA
AQP9, GADD45B, and TNFAIP3
AQP9, GADD45B, and SOCS1
AQP9, B4GALT5, and IL15RA
AQP9, B4GALT5, and TNFAIP3
AQP9, B4GALT5, and SOCS1
AQP9, IL15RA, and TNFAIP3
AQP9, IL15RA, and SOCS1
AQP9, TNFAIP3, and SOCS1
CDKN1A, GADD45B, and B4GALT5
CDKN1A, GADD45B, and IL15RA
CDKN1A, GADD45B, and TNFAIP3
CDKN1A, GADD45B, and SOCS1
CDKN1A, B4GALT5, and IL15RA
CDKN1A, B4GALT5, and TNFAIP3
CDKN1A, B4GALT5, and SOCS1
CDKN1A, IL15RA, and TNFAIP3
CDKN1A, IL15RA, and SOCS1
CDKN1A, TNFAIP3, and SOCS1
GADD45B, B4GALT5, and IL15RA
GADD45B, B4GALT5, and TNFAIP3
GADD45B, B4GALT5, and SOCS1
GADD45B, IL15RA, and TNFAIP3
GADD45B, IL15RA, and SOCS1
GADD45B, TNFAIP3, and SOCS1
B4GALT5, IL15RA, and TNFAIP3
B4GALT5, IL15RA, and SOCS1
B4GALT5, TNFAIP3, and SOCS1
IL15RA, TNFAIP3, and SOCS1

In some embodiments of the diagnostic methods described herein, the expression levels of a combination of four genes set forth in Table 1, such as any of the exemplary combinations shown in Table 4, may be determined.

TABLE 4
Exemplary Four-Gene Combinations of IRAK4 Biomarkers

CD38, SOCS3, AQP9, and CDKN1A
CD38, SOCS3, AQP9, and GADD45B
CD38, SOCS3, AQP9, and B4GALT5
CD38, SOCS3, AQP9, and IL15RA
CD38, SOCS3, AQP9 , and TNFAIP3
CD38, SOCS3, AQP9, and SOCS1
CD38, SOCS3, CDKN1A, and GADD45B
CD38, SOCS3, CDKN1A, and B4GALT5
CD38, SOCS3, CDKN1A, and IL15RA
CD38, SOCS3, CDKN1A, and TNFAIP3
CD38, SOCS3, CDKN1A, and SOCS1
CD38, SOCS3, GADD45B, and B4GALT5
CD38, SOCS3, GADD45B, and IL15RA
CD38, SOCS3, GADD45B, and TNFAIP3
CD38, SOCS3, GADD45B, and SOCS1
CD38, SOCS3, B4GALT5, and IL15RA
CD38, SOCS3, B4GALT5, and TNFAIP3
CD38, SOCS3, B4GALT5, and SOCS1
CD38, SOCS3, IL15RA, and TNFAIP3
CD38, SOCS3, IL15RA, and SOCS1
CD38, SOCS3, TNFAIP3, and SOCS1
CD38, AQP9, CDKN1A, and GADD45B
CD38, AQP9, CDKN1A, and B4GALT5
CD38, AQP9, CDKN1A, and IL15RA
CD38, AQP9, CDKN1A, and TNFAIP3
CD38, AQP9, CDKN1A, and SOCS1
CD38, AQP9, GADD45B, and B4GALT5
CD38, AQP9, GADD45B, and IL15RA
CD38, AQP9, GADD45B, and TNFAIP3
CD38, AQP9, GADD45B, and SOCS1
CD38, AQP9, B4GALT5, and IL15RA
CD38, AQP9, B4GALT5, and TNFAIP3
CD38, AQP9, B4GALT5, and SOCS1
CD38, AQP9, IL15RA, and TNFAIP3
CD38, AQP9, IL15RA, and SOCS1
CD38, AQP9, TNFAIP3, and SOCS1
CD38, CDKN1A, GADD45B, and B4GALT5
CD38, CDKN1A, GADD45B, and IL15RA
CD38, CDKN1A, GADD45B, and TNFAIP3
CD38, CDKN1A, GADD45B, and SOCS1
CD38, CDKN1A, B4GALT5, and IL15RA
CD38, CDKN1A, B4GALT5, and TNFAIP3
CD38, CDKN1A, B4GALT5, and SOCS1
CD38, CDKN1A, IL15RA, and TNFAIP3
CD38, CDKN1A, IL15RA, and SOCS1
CD38, CDKN1A, TNFAIP3, and SOCS1
CD38, GADD45B, B4GALT5, and IL15RA
CD38, GADD45B, B4GALT5, and TNFAIP3
CD38, GADD45B, B4GALT5, and SOCS1
CD38, GADD45B, IL15RA, and TNFAIP3
CD38, GADD45B, IL15RA, and SOCS1
CD38, GADD45B, TNFAIP3, and SOCS1
CD38, B4GALT5, IL15RA, and TNFAIP3
CD38, B4GALT5, IL15RA, and SOCS1
CD38, B4GALT5, TNFAIP3, and SOCS1
CD38, IL15RA, TNFAIP3, and SOCS1
SOCS3, AQP9, CDKN1A, and GADD45B
SOCS3, AQP9, CDKN1A, and B4GALT5
SOCS3, AQP9, CDKN1A, and IL15RA
SOCS3, AQP9, CDKN1A, and TNFAIP3
SOCS3, AQP9, CDKN1A, and SOCS1
SOCS3, AQP9, GADD45B, and B4GALT5
SOCS3, AQP9, GADD45B, and IL15RA
SOCS3, AQP9, GADD45B, and TNFAIP3
SOCS3, AQP9, GADD45B, and SOCS1
SOCS3, AQP9, B4GALT5, and IL15RA
SOCS3, AQP9, B4GALT5, and TNFAIP3
SOCS3, AQP9, B4GALT5, and SOCS1
SOCS3, AQP9, IL15RA, and TNFAIP3
SOCS3, AQP9, IL15RA, and SOCS1
SOCS3, AQP9, TNFAIP3, and SOCS1
SOCS3, CDKN1A, GADD45B, and B4GALT5
SOCS3, CDKN1A, GADD45B, and IL15RA
SOCS3, CDKN1A, GADD45B, and TNFAIP3
SOC53, CDKN1A, GADD45B, and SOCS1
SOCS3, CDKN1A, B4GALT5, and IL15RA
SOCS3, CDKN1A, B4GALT5, and TNFAIP3
SOCS3, CDKN1A, B4GALT5, and SOCS1
SOCS3, CDKN1A, IL15RA, and TNFAIP3
SOCS3, CDKN1A, IL15RA, and SOCS1
SOCS3, CDKN1A, TNFAIP3, and SOCS1
SOCS3, GADD45B, B4GALT5, and IL15RA
SOCS3, GADD45B, B4GALT5, and TNFAIP3
SOCS3, GADD45B, B4GALT5, and SOCS1
SOCS3, GADD45B, IL15RA, and TNFAIP3
SOCS3, GADD45B, IL15RA, and SOCS1
SOCS3, GADD45B, TNFAIP3, and SOCS1
SOCS3, B4GALT5, IL15RA, and TNFAIP3
SOCS3, B4GALT5, IL15RA, and SOCS1
SOCS3, B4GALT5, TNFAIP3, and SOCS1
SOCS3, IL15RA, TNFAIP3, and SOCS1
AQP9, CDKN1A, GADD45B, and B4GALT5
AQP9, CDKN1A, GADD45B, and IL15RA
AQP9, CDKN1A, GADD45B, and TNFAIP3
AQP9, CDKN1A, GADD45B, and SOCS1
AQP9, CDKN1A, B4GALT5, and IL15RA
AQP9, CDKN1A, B4GALT5, and TNFAIP3
AQP9, CDKN1A, B4GALT5, and SOCS1
AQP9, CDKN1A, IL15RA, and TNFAIP3
AQP9, CDKN1A, IL15RA, and SOCS1
AQP9, CDKN1A, TNFAIP3, and SOCS1
AQP9, GADD45B, B4GALT5, and IL15RA
AQP9, GADD45B, B4GALT5, and TNFAIP3
AQP9, GADD45B, B4GALT5, and SOCS1
AQP9, GADD45B, IL15RA, and TNFAIP3
AQP9, GADD45B, IL15RA, and SOCS1
AQP9, GADD45B, TNFAIP3, and SOCS1
AQP9, B4GALT5, IL15RA, and TNFAIP3
AQP9, B4GALT5, IL15RA, and SOCS1
AQP9, B4GALT5, TNFAIP3, and SOCS1
AQP9, IL15RA, TNFAIP3, and SOCS1
CDKN1A, GADD45B, B4GALT5, and IL15RA
CDKN1A, GADD45B, B4GALT5, and TNFAIP3
CDKN1A, GADD45B, B4GALT5, and SOCS1
CDKN1A, GADD45B, IL15RA, and TNFAIP3
CDKN1A, GADD45B, IL15RA, and SOCS1
CDKN1A, GADD45B, TNFAIP3, and SOCS1
CDKN1A, B4GALT5, IL15RA, and TNFAIP3
CDKN1A, B4GALT5, IL15RA, and SOCS1
CDKN1A, B4GALT5, TNFAIP3, and SOCS1
CDKN1A, IL15RA, TNFAIP3, and SOCS1
GADD45B, B4GALT5, IL15RA, and TNFAIP3
GADD45B, B4GALT5, IL15RA, and SOCS1
GADD45B, B4GALT5, TNFAIP3, and SOCS1
GADD45B, IL15RA, TNFAIP3, and SOCS1
B4GALT5, IL15RA, TNFAIP3, and SOCS1

In some embodiments of the diagnostic methods described herein, the expression levels of a combination of five genes set forth in Table 1, such as any of the exemplary combinations shown in Table 5, may be determined.

TABLE 5
Exemplary Five-Gene Combinations of IRAK4 Biomarkers

CD38, SOCS3, AQP9, CDKN1A, and GADD45B
CD38, SOCS3, AQP9, CDKN1A, and B4GALT5
CD38, SOCS3, AQP9, CDKN1A, and IL15RA
CD38, SOCS3, AQP9, CDKN1A, and TNFAIP3
CD38, SOCS3, AQP9, CDKN1A, and SOCS1
CD38, SOCS3, AQP9, GADD45B, and B4GALT5
CD38, SOCS3, AQP9, GADD45B, and IL15RA
CD38, SOCS3, AQP9, GADD45B, and TNFAIP3
CD38, SOCS3, AQP9, GADD45B, and SOCS1
CD38, SOCS3, AQP9, B4GALT5, and IL15RA
CD38, SOCS3, AQP9, B4GALT5, and TNFAIP3
CD38, SOCS3, AQP9, B4GALT5, and SOCS1
CD38, SOCS3, AQP9, IL15RA, and TNFAIP3
CD38, SOCS3, AQP9, IL15RA, and SOCS1
CD38, SOCS3, AQP9, TNFAIP3, and SOCS1
CD38, SOCS3, CDKN1A, GADD45B, and B4GALT5
CD38, SOCS3, CDKN1A, GADD45B, and IL15RA
CD38, SOCS3, CDKN1A, GADD45B, and TNFAIP3
CD38, SOCS3, CDKN1A, GADD45B, and SOCS1
CD38, SOCS3, CDKN1A, B4GALT5, and IL15RA
CD38, SOCS3, CDKN1A, B4GALT5, and TNFAIP3
CD38, SOCS3, CDKN1A, B4GALT5, and SOCS1
CD38, SOCS3, CDKN1A, IL15RA, and TNFAIP3
CD38, SOCS3, CDKN1A, IL15RA, and SOCS1
CD38, SOCS3, CDKN1A, TNFAIP3, and SOCS1
CD38, SOCS3, GADD45B, B4GALT5, and IL15RA
CD38, SOCS3, GADD45B, B4GALT5, and TNFAIP3
CD38, SOCS3, GADD45B, B4GALT5, and SOCS1
CD38, SOCS3, GADD45B, IL15RA, and TNFAIP3
CD38, SOCS3, GADD45B, IL15RA, and SOCS1
CD38, SOCS3, GADD45B, TNFAIP3, and SOCS1
CD38, SOCS3, B4GALT5, IL15RA, and TNFAIP3
CD38, SOCS3, B4GALT5, IL15RA, and SOCS1
CD38, SOCS3, B4GALT5, TNFAIP3, and SOCS1
CD38, SOCS3, IL15RA, TNFAIP3, and SOCS1
CD38, AQP9, CDKN1A, GADD45B, and B4GALT5
CD38, AQP9, CDKN1A, GADD45B, and IL15RA
CD38, AQP9, CDKN1A, GADD45B, and TNFAIP3
CD38, AQP9, CDKN1A, GADD45B, and SOCS1
CD38, AQP9, CDKN1A, B4GALT5, and IL15RA
CD38, AQP9, CDKN1A, B4GALT5, and TNFAIP3
CD38, AQP9, CDKN1A, B4GALT5, and SOCS1
CD38, AQP9, CDKN1A, IL15RA, and TNFAIP3
CD38, AQP9, CDKN1A, IL15RA, and SOCS1
CD38, AQP9, CDKN1A, TNFAIP3, and SOCS1
CD38, AQP9, GADD45B, B4GALT5, and IL15RA
CD38, AQP9, GADD45B, B4GALT5, and TNFAIP3
CD38, AQP9, GADD45B, B4GALT5, and SOCS1
CD38, AQP9, GADD45B, IL15RA, and TNFAIP3
CD38, AQP9, GADD45B, IL15RA, and SOCS1
CD38, AQP9, GADD45B, TNFAIP3, and SOCS1
CD38, AQP9, B4GALT5, IL15RA, and TNFAIP3
CD38, AQP9, B4GALT5, IL15RA, and SOCS1
CD38, AQP9, B4GALT5, TNFAIP3, and SOCS1
CD38, AQP9, IL15RA, TNFAIP3, and SOCS1
CD38, CDKN1A, GADD45B, B4GALT5, and IL15RA
CD38, CDKN1A, GADD45B, B4GALT5, and TNFAIP3
CD38, CDKN1A, GADD45B, B4GALT5, and SOCS1
CD38, CDKN1A, GADD45B, IL15RA, and TNFAIP3
CD38, CDKN1A, GADD45B, IL15RA, and SOCS1
CD38, CDKN1A, GADD45B, TNFAIP3, and SOCS1
CD38, CDKN1A, B4GALT5, IL15RA, and TNFAIP3
CD38, CDKN1A, B4GALT5, IL15RA, and SOCS1
CD38, CDKN1A, B4GALT5, TNFAIP3, and SOCS1
CD38, CDKN1A, IL15RA, TNFAIP3, and SOCS1
CD38, GADD45B, B4GALT5, IL15RA, and TNFAIP3
CD38, GADD45B, B4GALT5, IL15RA, and SOCS1
CD38, GADD45B, B4GALT5, TNFAIP3, and SOCS1
CD38, GADD45B, IL15RA, TNFAIP3, and SOCS1
CD38, B4GALT5, IL15RA, TNFAIP3, and SOCS1
SOCS3, AQP9, CDKN1A, GADD45B, and B4GALT5
SOCS3, AQP9, CDKN1A, GADD45B, and IL15RA
SOCS3, AQP9, CDKN1A, GADD45B, and TNFAIP3
SOCS3, AQP9, CDKN1A, GADD45B, and SOCS1
SOCS3, AQP9, CDKN1A, B4GALT5, and IL15RA
SOCS3, AQP9, CDKN1A, B4GALT5, and TNFAIP3
SOCS3, AQP9, CDKN1A, B4GALT5, and SOCS1
SOCS3, AQP9, CDKN1A, IL15RA, and TNFAIP3
SOCS3, AQP9, CDKN1A, IL15RA, and SOCS1
SOCS3, AQP9, CDKN1A, TNFAIP3, and SOCS1
SOCS3, AQP9, GADD45B, B4GALT5, and IL15RA
SOCS3, AQP9, GADD45B, B4GALT5, and TNFAIP3
SOCS3, AQP9, GADD45B, B4GALT5, and SOCS1
SOCS3, AQP9, GADD45B, IL15RA, and TNFAIP3
SOCS3, AQP9, GADD45B, IL15RA, and SOCS1
SOCS3, AQP9, GADD45B, TNFAIP3, and SOCS1
SOCS3, AQP9, B4GALT5, IL15RA, and TNFAIP3
SOC53, AQP9, B4GALT5, IL15RA, and SOCS1
SOCS3, AQP9, B4GALT5, TNFAIP3, and SOCS1
SOCS3, AQP9, IL15RA, TNFAIP3, and SOCS1
SOCS3, CDKN1A, GADD45B, B4GALT5, and IL15RA
SOCS3, CDKN1A, GADD45B, B4GALT5, and TNFAIP3
SOCS3, CDKN1A, GADD45B, B4GALT5, and SOCS1
SOCS3, CDKN1A, GADD45B, IL15RA, and TNFAIP3
SOCS3, CDKN1A, GADD45B, IL15RA, and SOCS1
SOCS3, CDKN1A, GADD45B, TNFAIP3, and SOCS1
SOCS3, CDKN1A, B4GALT5, IL15RA, and TNFAIP3
SOCS3, CDKN1A, B4GALT5, IL15RA, and SOCS1
SOCS3, CDKN1A, B4GALT5, TNFAIP3, and SOCS1
SOCS3, CDKN1A, IL15RA, TNFAIP3, and SOCS1
SOCS3, GADD45B, B4GALT5, IL15RA, and TNFAIP3
SOCS3, GADD45B, B4GALT5, IL15RA, and SOCS1
SOCS3, GADD45B, B4GALT5, TNFAIP3, and SOCS1
SOCS3, GADD45B, IL15RA, TNFAIP3, and SOCS1
SOCS3, B4GALT5, IL15RA, TNFAIP3, and SOCS1
AQP9, CDKN1A, GADD45B, B4GALT5, and IL15RA
AQP9, CDKN1A, GADD45B, B4GALT5, and TNFAIP3
AQP9, CDKN1A, GADD45B, B4GALT5, and SOCS1
AQP9, CDKN1A, GADD45B, IL15RA, and TNFAIP3
AQP9, CDKN1A, GADD45B, IL15RA, and SOCS1
AQP9, CDKN1A, GADD45B, TNFAIP3, and SOCS1
AQP9, CDKN1A, B4GALT5, IL15RA, and TNFAIP3
AQP9, CDKN1A, B4GALT5, IL15RA, and SOCS1
AQP9, CDKN1A, B4GALT5, TNFAIP3, and SOCS1
AQP9, CDKN1A, IL15RA, TNFAIP3, and SOCS1
AQP9, GADD45B, B4GALT5, IL15RA, and TNFAIP3
AQP9, GADD45B, B4GALT5, IL15RA, and SOCS1
AQP9, GADD45B, B4GALT5, TNFAIP3, and SOCS1
AQP9, GADD45B, IL15RA, TNFAIP3, and SOCS1
AQP9, B4GALT5, IL15RA, TNFAIP3, and SOCS1
CDKN1A, GADD45B, B4GALT5, IL15RA, and TNFAIP3
CDKN1A, GADD45B, B4GALT5, IL15RA, and SOCS1
CDKN1A, GADD45B, B4GALT5, TNFAIP3, and SOCS1
CDKN1A, GADD45B, IL15RA, TNFAIP3, and SOCS1
CDKN1A, B4GALT5, IL15RA, TNFAIP3, and SOCS1
GADD45B, B4GALT5, IL15RA, TNFAIP3, and SOCS1

In some embodiments of the diagnostic methods described herein, the expression levels of a combination of six genes set forth in Table 1, such as any of the exemplary combinations shown in Table 6, may be determined.

TABLE 6
Exemplary Six-Gene Combinations of IRAK4 Biomarkers

CD38, SOCS3, AQP9, CDKN1A, GADD45B, and B4GALT5
CD38, SOCS3, AQP9, CDKN1A, GADD45B, and IL15RA
CD38, SOCS3, AQP9, CDKN1A, GADD45B, and TNFAIP3
CD38, SOCS3, AQP9, CDKN1A, GADD45B, and SOCS1
CD38, SOCS3, AQP9, CDKN1A, B4GALT5, and IL15RA
CD38, SOCS3, AQP9, CDKN1A, B4GALT5, and TNFAIP3
CD38, SOCS3, AQP9, CDKN1A, B4GALT5, and SOCS1
CD38, SOCS3, AQP9, CDKN1A, IL15RA, and TNFAIP3
CD38, SOCS3, AQP9, CDKN1A, IL15RA, and SOCS1
CD38, SOCS3, AQP9, CDKN1A, TNFAIP3, and SOCS1
CD38, SOCS3, AQP9, GADD45B, B4GALT5, and IL15RA
CD38, SOCS3, AQP9, GADD45B, B4GALT5, and TNFAIP3
CD38, SOCS3, AQP9, GADD45B, B4GALT5, and SOCS1
CD38, SOCS3, AQP9, GADD45B, IL15RA, and TNFAIP3
CD38, SOCS3, AQP9, GADD45B, IL15RA, and SOCS1
CD38, SOCS3, AQP9, GADD45B, TNFAIP3, and SOCS1
CD38, SOCS3, AQP9, B4GALT5, IL15RA, and TNFAIP3
CD38, SOCS3, AQP9, B4GALT5, IL15RA, and SOCS1
CD38, SOCS3, AQP9, B4GALT5, TNFAIP3, and SOCS1
CD38, SOCS3, AQP9, IL15RA, TNFAIP3, and SOCS1
CD38, SOCS3, CDKN1A, GADD45B, B4GALT5, and IL15RA
CD38, SOCS3, CDKN1A, GADD45B, B4GALT5, and TNFAIP3
CD38, SOCS3, CDKN1A, GADD45B, B4GALT5, and SOCS1
CD38, SOCS3, CDKN1A, GADD45B, IL15RA, and TNFAIP3
CD38, SOCS3, CDKN1A, GADD45B, IL15RA, and SOCS1
CD38, SOCS3, CDKN1A, GADD45B, TNFAIP3, and SOCS1
CD38, SOCS3, CDKN1A, B4GALT5, IL15RA, and TNFAIP3
CD38, SOCS3, CDKN1A, B4GALT5, IL15RA, and SOCS1
CD38, SOCS3, CDKN1A, B4GALT5, TNFAIP3, and SOCS1
CD38, SOCS3, CDKN1A, IL15RA, TNFAIP3, and SOCS1
CD38, SOCS3, GADD45B, B4GALT5, IL15RA, and TNFAIP3
CD38, SOCS3, GADD45B, B4GALT5, IL15RA, and SOCS1
CD38, SOCS3, GADD45B, B4GALT5, TNFAIP3, and SOCS1
CD38, SOCS3, GADD45B, IL15RA, TNFAIP3, and SOCS1
CD38, SOCS3, B4GALT5, IL15RA, TNFAIP3, and SOCS1
CD38, AQP9, CDKN1A, GADD45B, B4GALT5, and IL15RA
CD38, AQP9, CDKN1A, GADD45B, B4GALT5, and TNFAIP3
CD38, AQP9, CDKN1A, GADD45B, B4GALT5, and SOCS1
CD38, AQP9, CDKN1A, GADD45B, IL15RA, and TNFAIP3
CD38, AQP9, CDKN1A, GADD45B, IL15RA, and SOCS1
CD38, AQP9, CDKN1A, GADD45B, TNFAIP3, and SOCS1
CD38, AQP9, CDKN1A, B4GALT5, IL15RA, and TNFAIP3
CD38, AQP9, CDKN1A, B4GALT5, IL15RA, and SOCS1
CD38, AQP9, CDKN1A, B4GALT5, TNFAIP3, and SOCS1
CD38, AQP9, CDKN1A, IL15RA, TNFAIP3, and SOCS1
CD38, AQP9, GADD45B, B4GALT5, IL15RA, and TNFAIP3
CD38, AQP9, GADD45B, B4GALT5, IL15RA, and SOCS1
CD38, AQP9, GADD45B, B4GALT5, TNFAIP3, and SOCS1
CD38, AQP9, GADD45B, IL15RA, TNFAIP3, and SOCS1
CD38, AQP9, B4GALT5, IL15RA, TNFAIP3, and SOCS1
CD38, CDKN1A, GADD45B, B4GALT5, IL15RA, and TNFAIP3
CD38, CDKN1A, GADD45B, B4GALT5, IL15RA, and SOCS1
CD38, CDKN1A, GADD45B, B4GALT5, TNFAIP3, and SOCS1
CD38, CDKN1A, GADD45B, IL15RA, TNFAIP3, and SOCS1
CD38, CDKN1A, B4GALT5, IL15RA, TNFAIP3, and SOCS1
CD38, GADD45B, B4GALT5, IL15RA, TNFAIP3, and SOCS1
SOCS3, AQP9, CDKN1A, GADD45B, B4GALT5, and IL15RA
SOCS3, AQP9, CDKN1A, GADD45B, B4GALT5, and TNFAIP3
SOCS3, AQP9, CDKN1A, GADD45B, B4GALT5, and SOCS1
SOCS3, AQP9, CDKN1A, GADD45B, IL15RA, and TNFAIP3
SOCS3, AQP9, CDKN1A, GADD45B, IL15RA, and SOCS1
SOCS3, AQP9, CDKN1A, GADD45B TNFAIP3, and SOCS1
SOCS3, AQP9, CDKN1A, B4GALT5, IL15RA, and TNFAIP3
SOCS3, AQP9, CDKN1A, B4GALT5, IL15RA, and SOCS1
SOCS3, AQP9, CDKN1A, B4GALT5, TNFAIP3, and SOCS1
SOCS3, AQP9, CDKN1A, IL15RA, TNFAIP3, and SOCS1
SOCS3, AQP9, GADD45B, B4GALT5, IL15RA, and TNFAIP3
SOCS3, AQP9, GADD45B, B4GALT5, IL15RA, and SOCS1
SOCS3, AQP9, GADD45B, B4GALT5, TNFAIP3, and SOCS1
SOCS3, AQP9, GADD45B, IL15RA, TNFAIP3, and SOCS1
SOCS3, AQP9, B4GALT5, IL15RA, TNFAIP3, and SOCS1
SOCS3, CDKN1A, GADD45B, B4GALT5, IL15RA, and TNFAIP3
SOCS3, CDKN1A, GADD45B, B4GALT5, IL15RA, and SOCS1
SOCS3, CDKN1A, GADD45B, B4GALT5, TNFAIP3, and SOCS1
SOCS3, CDKN1A, GADD45B, IL15RA, TNFAIP3, and SOCS1
SOCS3, CDKN1A, B4GALT5, IL15RA, TNFAIP3, and SOCS1
SOCS3, GADD45B, B4GALT5, IL15RA, TNFAIP3, and SOCS1
AQP9, CDKN1A, GADD45B, B4GALT5, IL15RA, and TNFAIP3
AQP9, CDKN1A, GADD45B, B4GALT5, IL15RA, and SOCS1
AQP9, CDKN1A, GADD45B, B4GALT5, TNFAIP3, and SOCS1
AQP9, CDKN1A, GADD45B, IL15RA, TNFAIP3, and SOCS1
AQP9, CDKN1A, B4GALT5, IL15RA, TNFAIP3, and SOCS1
AQP9, GADD45B, B4GALT5, IL15RA, TNFAIP3, and SOCS1
CDKN1A, GADD45B, B4GALT5, IL15RA, TNFAIP3, and SOCS1

In some embodiments of the diagnostic methods described herein, the expression levels of a combination of seven genes set forth in Table 1, such as any of the exemplary combinations shown in Table 7, may be determined.

TABLE 7
Exemplary Seven-Gene Combinations of IRAK4 Biomarkers

CD38, SOCS3, AQP9, CDKN1A, GADD45B, B4GALT5, and IL15RA
CD38, SOCS3, AQP9, CDKN1A, GADD45B, B4GALT5, and TNFAIP3
CD38, SOCS3, AQP9, CDKN1A, GADD45B, B4GALT5, and SOCS1
CD38, SOCS3, AQP9, CDKN1A, GADD45B, IL15RA, and TNFAIP3
CD38, SOCS3, AQP9, CDKN1A, GADD45B, IL15RA, and SOCS1
CD38, SOCS3, AQP9, CDKN1A, GADD45B, TNFAIP3, and SOCS1
CD38, SOCS3, AQP9, CDKN1A, B4GALT5, IL15RA, and TNFAIP3
CD38, SOCS3, AQP9, CDKN1A, B4GALT5, IL15RA, and SOCS1
CD38, SOCS3, AQP9, CDKN1A, B4GALT5, TNFAIP3, and SOCS1
CD38, SOCS3, AQP9, CDKN1A, IL15RA, TNFAIP3, and SOCS1
CD38, SOCS3, AQP9, GADD45B, B4GALT5, IL15RA, and TNFAIP3
CD38, SOCS3, AQP9, GADD45B, B4GALT5, IL15RA, and SOCS1
CD38, SOCS3, AQP9, GADD45B, B4GALT5, TNFAIP3, and SOCS1
CD38, SOCS3, AQP9, GADD45B, IL15RA, TNFAIP3, and SOCS1
CD38, SOCS3, AQP9, B4GALT5, IL15RA, TNFAIP3, and SOCS1
CD38, SOCS3, CDKN1A, GADD45B, B4GALT5, IL15RA, and
TNFAIP3
CD38, SOCS3, CDKN1A, GADD45B, B4GALT5, IL15RA, and SOCS1
CD38, SOCS3, CDKN1A, GADD45B, B4GALT5, TNFAIP3, and
SOCS1
CD38, SOCS3, CDKN1A, GADD45B, IL15RA, TNFAIP3, and SOCS1
CD38, SOCS3, CDKN1A, B4GALT5, IL15RA, TNFAIP3, and SOCS1
CD38, SOCS3, GADD45B, B4GALT5, IL15RA, TNFAIP3, and SOCS1
CD38, AQP9, CDKN1A, GADD45B, B4GALT5, IL15RA, and TNFAIP3
CD38, AQP9, CDKN1A, GADD45B, B4GALT5, IL15RA, and SOCS1
CD38, AQP9, CDKN1A, GADD45B, B4GALT5, TNFAIP3, and SOCS1
CD38, AQP9, CDKN1A, GADD45B, IL15RA, TNFAIP3, and SOCS1
CD38, AQP9, CDKN1A, B4GALT5, IL15RA, TNFAIP3, and SOCS1
CD38, AQP9, GADD45B, B4GALT5, IL15RA, TNFAIP3, and SOCS1
CD38, CDKN1A, GADD45B, B4GALT5, IL15RA, TNFAIP3, and
SOCS1
SOCS3, AQP9, CDKN1A, GADD45B, B4GALT5, IL15RA, and
TNFAIP3
SOCS3, AQP9, CDKN1A, GADD45B, B4GALT5, IL15RA, and SOCS1
SOCS3, AQP9, CDKN1A, GADD45B, B4GALT5, TNFAIP3, and
SOCS1
SOCS3, AQP9, CDKN1A, GADD45B, IL15RA, TNFAIP3, and SOCS1
SOCS3, AQP9, CDKN1A, B4GALT5, IL15RA, TNFAIP3, and SOCS1
SOCS3, AQP9, GADD45B, B4GALT5, IL15RA, TNFAIP3, and SOCS1
SOCS3, CDKN1A, GADD45B, B4GALT5, IL15RA, TNFAIP3, and
SOCS1
AQP9, CDKN1A, GADD45B, B4GALT5, IL15RA, TNFAIP3, and
SOCS1

In some embodiments of the diagnostic methods described herein, the expression levels of a combination of eight genes set forth in Table 1, such as any of the exemplary combinations shown in Table 8, may be determined.

TABLE 8
Exemplary Eight-Gene Combinations of IRAK4 Biomarkers

CD38, SOCS3, AQP9, CDKN1A, GADD45B, B4GALT5, IL15RA, and
TNFAIP3
CD38, SOCS3, AQP9, CDKN1A, GADD45B, B4GALT5, IL15RA, and
SOCS1
CD38, SOCS3, AQP9, CDKN1A, GADD45B, B4GALT5, TNFAIP3, and
SOCS1
CD38, SOCS3, AQP9, CDKN1A, GADD45B, IL15RA, TNFAIP3, and
SOCS1
CD38, SOCS3, AQP9, CDKN1A, B4GALT5, IL15RA, TNFAIP3, and
SOCS1
CD38, SOCS3, AQP9, GADD45B, B4GALT5, IL15RA, TNFAIP3, and
SOCS1
CD38, SOCS3, CDKN1A, GADD45B, B4GALT5, IL15RA, TNFAIP3,
and SOCS1
CD38, AQP9, CDKN1A, GADD45B, B4GALT5, IL15RA, TNFAIP3,
and SOCS1
SOCS3, AQP9, CDKN1A, GADD45B, B4GALT5, IL15RA, TNFAIP3,
and SOCS1

The presence and/or expression level (amount) of the IRAK4 pathway biomarkers described herein in a sample can be analyzed by a number of methodologies, many of which are known in the art and understood by the skilled artisan, including, but not limited to, immunohistochemistry (“IHC”), Western blot analysis, immunoprecipitation, molecular binding assays, enzyme-linked immunosorbent assay (ELISA), enzyme-linked immunofiltration assay (ELIFA), fluorescence activated cell sorting (“FACS”), MassARRAY, proteomics, quantitative blood based assays (e.g., serum ELISA), biochemical enzymatic activity assays, in situ hybridization, fluorescence in situ hybridization (FISH), Southern analysis, Northern analysis, whole genome sequencing, polymerase chain reaction (PCR) (including quantitative PCR (qPCR)) and other amplification type detection methods, such as, for example, branched DNA, SISBA, TMA and the like), RNA-Seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In any of the preceding methods, the presence and/or expression level (amount) of a biomarker (e.g., CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and/or ACSL1) may be a nucleic acid expression level. In some instances, the nucleic acid expression level is determined using qPCR, RT-PCR, RNA-Seq, multiplex qPCR or RT-qPCR, microarray analysis, SAGE, MassARRAY technique, or in situ hybridization (e.g., FISH).

In a particular instance, the expression level of a biomarker (e.g., CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and/or ACSL1) is an mRNA expression level. Methods for the evaluation of mRNAs in cells are well known and include, for example, qPCR, RNA-Seq (e.g., whole transcriptome shotgun sequencing) using next generation sequencing techniques, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like). In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined. Optional methods include protocols that examine or detect mRNAs, such as target mRNAs, in a tissue or cell sample by microarray technologies. Using nucleic acid microarrays test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of treatment including an IRAK4 pathway inhibitor may be arrayed on a solid support. Hybridization of a labeled probe with a particular array member indicates that the sample from which the probe was derived expresses that gene.

In any of the preceding methods, the presence and/or expression level (amount) of a biomarker (e.g., CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and/or ACSL1) is measured by determining protein expression levels of the biomarker. In certain instances, the method comprises contacting the biological sample with antibodies that specifically bind to a biomarker described herein under conditions permissive for binding of the biomarker, and detecting whether a complex is formed between the antibodies and biomarker. Such method may be an in vitro or in vivo method. Any method of measuring protein expression levels known in the art may be used. For example, in some instances, a protein expression level of a biomarker (e.g., CD38, SOCS3, AQP9, CDKN1A, GADD45B, B4GALT5, IL15RA, TNFAIP3, SOCS1, IL1RN, PFKFB3, BCL2A1, CXCL10, CCL8, GPR84, C15orf48, DRAM1, CXCL11, TNFAIP6, CSRNP1, PLSCR1, CLEC4E, SAMSN1, and/or ACSL1) is determined using a method selected from the group consisting of flow cytometry (e.g., fluorescence-activated cell sorting (FACS™)), Western blot, ELISA, ELIFA, immunoprecipitation, immunohistochemistry (IHC), immunofluorescence, radioimmunoassay, dot blotting, immunodetection methods, HPLC, surface plasmon resonance, optical spectroscopy, mass spectrometry, and HPLC.

In certain instances, a reference level, reference sample, reference cell, reference tissue, control sample, control cell, or control tissue is a single sample or a combination of multiple samples from the same subject or individual that are obtained at one or more different time points than when the test sample is obtained. For example, a reference level, reference sample, reference cell, reference tissue, control sample, control cell, or control tissue is obtained at an earlier time point from the same subject or individual than when the test sample is obtained. Such reference level, reference sample, reference cell, reference tissue, control sample, control cell, or control tissue may be useful if the reference sample is obtained during initial diagnosis of an IRAK4-mediated disorder or condition and the test sample is later obtained when the IRAK4-mediated disorder or condition becomes more severe.

In certain embodiments, a reference level, reference sample, reference cell, reference tissue, control sample, control cell, or control tissue is a combination of multiple samples from one or more healthy individuals who are not the patient. In certain embodiments, a reference level, reference sample, reference cell, reference tissue, control sample, control cell, or control tissue is a combination of multiple samples from one or more individuals with a disease or disorder (e.g., an IRAK4-mediated disorder or condition) who are not the patient or individual. In certain embodiments, a reference level, reference sample, reference cell, reference tissue, control sample, control cell, or control tissue is pooled RNA samples from normal tissues or pooled plasma or serum samples from one or more individuals who are not the patient. In certain embodiments, a reference level, reference sample, reference cell, reference tissue, control sample, control cell, or control tissue is pooled RNA samples from pooled plasma or serum samples from one or more individuals with an IRAK4-mediated disorder or condition (e.g., an inflammatory disorder or an immune disorder) who are not the patient. In certain embodiments, the reference level is the median level of expression of a biomarker across a set of samples. In certain embodiments, the reference level is the median level of expression of a biomarker across a population of patients having a particular disease or disorder (e.g., an IRAK4-mediated disorder or condition (e.g., an inflammatory disorder or an immune disorder)).

In any of the preceding diagnostic methods, the IRAK4-mediated disorder or condition is selected from the group consisting of an immune disorder, an inflammatory disorder, a fibrotic disorder, an eosinophilic disorder, an infection, pain, a central nervous system disorder, an acute kidney injury, a chronic kidney disease, endometriosis, non-alcoholic fatty liver disease, a metabolic syndrome, and obesity.

In some instances, the immune disorder is allergic airway syndrome, allergic rhinitis, allograft rejection, asthma, atopic dermatitis, contact dermatitis, Crohn's disease, cutaneous lupus, delayed hypersensitivity, diabetes, gout, graft versus host disease, graft rejection, inflammatory bowel disease (IBD), inflammatory myositis (e.g., polymyositis, dermatomyositis), lupus, lupus nephritis, multiple sclerosis, psoriasis, rheumatoid arthritis, scleroderma, sepsis, systemic lupus erythematosus, systemic sclerosis, or ulcerative colitis.

In some instances, the inflammatory disorder is acute respiratory distress syndrome, acute lung injury, adult onset Still's disease, allergic airway syndrome, allergic rhinitis, asthma, atherosclerosis, atopic dermatitis, bronchitis, calcium pyrophosphate deposition disease (CPPD), cerebrovascular accident (e.g., stroke), chronic obstructive pulmonary disease (COPD), contact dermatitis, Crohn's disease, cryopyrin-associated periodic syndromes (CAPS), cutaneous lupus, delayed hypersensitivity, gout, graft versus host disease, inflammatory bowel disease (IBD), inflammatory myositis (e.g., polymyositis, dermatomyositis), lupus, lupus nephritis, rheumatoid arthritis, rhinitis, scleroderma, sepsis, systemic lupus erythematosus, systemic onset juvenile idiopathic arthritis, systemic sclerosis, or ulcerative colitis.

In some instances, the eosinophilic disorder is allergic rhinitis, asthma, atopic dermatitis, chronic obstructive pulmonary disease (COPD), or contact dermatitis.

In some instances, the fibrotic disorder is atherosclerosis, scleroderma, or systemic sclerosis.

In some instances, the central nervous system disorder is cerebrovascular accident (e.g., stroke), multiple sclerosis, or neurodegeneration.

In some instances, the pain is neuropathic pain.

In some instances, the infection is bronchitis or sepsis.

In any of the preceding diagnostic methods of the invention, the IRAK4 pathway inhibitor is an IRAK4 inhibitor, an IRAK1 inhibitor, a toll-like receptor (TLR) inhibitor, an interleukin-1 receptor (IL-1R) inhibitor, an interleukin-33 receptor (IL-33R) inhibitor, or a myeloid differentiation primary response gene 88 (MyD88) inhibitor. In one instance, the IRAK4 pathway inhibitor is an IRAK4 inhibitor. In some instances, the IRAK4 pathway inhibitor is a TLR inhibitor. In some instances, the TLR inhibitor is a TLR7 inhibitor, a TLR8 inhibitor, a TLR9 inhibitor, a TLR1 inhibitor, a TLR2 inhibitor, a TLR4 inhibitor, a TLRS inhibitor, a TLR6 inhibitor, or a TLR10 inhibitor. In certain instances, the IRAK4 pathway inhibitor is a TLR7 inhibitor, a TLR8 inhibitor, or both a TLR7 and TLR8 inhibitor. In certain instances, the IRAK4 pathway inhibitor is a TLR9 inhibitor. In any one of the preceding embodiments, the IRAK4 pathway inhibitor is a small molecule inhibitor. In other embodiments, the IRAK4 pathway inhibitor is a protein or multi-protein complex, such as an antibody.

B. Methods of Treatment with IRAK4 Pathway Inhibitors

The present invention also provides methods for treating a patient having IRAK4-mediated disorders or conditions (e.g., immune disorders (e.g., systemic lupus erythematosus (SLE)) or inflammatory disorders (e.g., asthma)). Accordingly, in some instances, the methods of the invention include administering to the patient an IRAK4 pathway inhibitor. Any of the IRAK4 pathway inhibitors described above or known in the art may be used in connection with the methods.

In one aspect, the invention features a method of treating a patient having an IRAK4-mediated disorder or condition with an IRAK4 pathway inhibitor, the method (a) determining, in a sample obtained from the patient at a time point following administration of a first dose of the IRAK4 pathway inhibitor, the expression level of one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1; (b) comparing the expression level of the one or more genes set forth in Table 1 in the sample with a reference expression level; and (c) administering at least a second dose of the IRAK4 pathway inhibitor to the patient based on a decreased expression level of the one or more genes set forth in Table 1 relative to the reference expression level.

In some instances, the expression level of the one or more genes set forth in Table 1 is decreased in the sample obtained from the patient at a time point following administration of a first dose of the IRAK4 pathway inhibitor relative to the reference expression level. For example, in some instances, the expression level of the one or more genes set forth in Table 1 is decreased by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, or about 100%), e.g., from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%, relative to a reference expression level (e.g., the expression level of the one or more genes set forth in Table 1 in a sample from the patient obtained prior to administration of the first dose of the IRAK4 pathway inhibitor). In other instances, the expression level of the one or more genes set forth in Table 1 is decreased by about 0.5-fold, about 0.6-fold, about 0.7-fold, about 0.8-fold, about 0.9-fold, about 1-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, or about 10-fold or greater, e.g., from about 0.5-fold to about 0.7-fold, from about 0.7-fold to about 1-fold, from about 1-fold to about 1.5-fold, from about 1.5-fold to about 2-fold, from about 2-fold to about 3-fold, from about 3-fold to about 4-fold, from about 4-fold to about 5-fold, from about 5-fold to about 6-fold, from about 6-fold to about 7-fold, from about 7-fold to about 8-fold, or from about 9-fold to about 10-fold or greater, relative to the reference expression level (e.g., the expression level of the one or more genes set forth in Table 1 in a sample from the patient obtained prior to administration of the first dose of the IRAK4 pathway inhibitor).

In some embodiments, reduced or decreased expression refers to an overall reduction of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference level, reference sample, reference cell, reference tissue, control sample, control cell, or control tissue. In certain embodiments, reduced expression refers to the decrease in expression level (amount) of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level (amount) of the respective biomarker in a reference level, reference sample, reference cell, reference tissue, control sample, control cell, or control tissue.

In certain instances, the method includes administering to a patient having an IRAK4-mediated disorder or condition who has already received a first dose of an IRAK4 pathway inhibitor, at least a second dose of the IRAK4 pathway inhibitor based on a decreased expression level of the one or more genes set forth in Table 1 relative to the reference expression level. In some instances, the method includes administering to a patient having an IRAK4-mediated disorder or condition who has already received a first dose of an IRAK4 pathway inhibitor, at least a second dose of the IRAK4 pathway inhibitor based on a decreased expression level of one or more genes (e.g., one gene, two genes, three genes, four genes, five genes, six genes, seven genes, eight genes, nine genes, ten genes, eleven genes, or all twelve genes) selected from the group consisting of CD38, SOCS3, AQP9, CDKN1A, GADD45B, B4GALT5, IL15RA, TNFAIP3, SOCS1, IL1RN, PFKFB3, and BCL2A1 relative to a reference expression level. In some instances, the method includes administering to a patient having an IRAK4-mediated disorder or condition who has already received a first dose of an IRAK4 pathway inhibitor, at least a second dose of the IRAK4 pathway inhibitor based on a decreased expression level of one or more genes (e.g., one gene, two genes, three genes, four genes, five genes, six genes, seven genes, eight genes, nine genes, ten genes, or all eleven genes) selected from the group consisting of CD38, SOCS3, AQP9, CDKN1A, GADD45B, B4GALT5, IL15RA, TNFAIP3, SOCS1, IL1RN, and PFKFB3 relative to a reference expression level. In other instances, the method includes administering to a patient having an IRAK4-mediated disorder or condition who has already received a first dose of an IRAK4 pathway inhibitor, at least a second dose of the IRAK4 pathway inhibitor based on a decreased expression level of one or more genes (e.g., one gene, two genes, three genes, four genes, five genes, six genes, seven genes, eight genes, or all nine genes) selected from the group consisting of CD38, SOCS3, AQP9, CDKN1A, GADD45B, B4GALT5, IL15RA, TNFAIP3, and SOCS1 relative to a reference expression level.

In any one the preceding embodiments, the reference expression level can be: (i) the expression level of the one or more genes set forth in Table 1 in a sample from the patient obtained prior to administration of the first dose of the IRAK4 pathway inhibitor; (ii) the expression level of the one or more genes set forth in Table 1 in a reference population (e.g., a median expression level of the one or more genes set forth in Table 1 in a reference population); (iii) a pre-assigned expression level for the one or more genes set forth in Table 1; (iv) the expression level of the one or more genes set forth in Table 1 in a sample obtained from the patient at a previous time point, wherein the previous time point is following administration of the first dose of the IRAK4 pathway inhibitor; or (v) the expression level of the one or more genes set forth in Table 1 in a sample obtained from the patient at a subsequent time point. In another aspect, the invention features a therapeutic method of treating a patient having an IRAK4-mediated disorder or condition, the method comprising administering to the patient an IRAK4 pathway inhibitor, wherein prior to treatment the expression level of one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1 in a sample obtained from the patient has been determined to be increased relative to a reference expression level. In some instances, the expression level of one or more genes (e.g., 1, 2, 3, 4, 5, 6, 7, 8, 9, 10, 11, 12, 13, 14, 15, 16, 17, 18, 19, 20, 21, 22, 23, or all 24 genes) set forth in Table 1 has been determined to be increased in the sample obtained from the patient relative to the reference expression level. For example, in some instances, the expression level of the one or more genes set forth in Table 1 has been determined to be increased by about 1% or more (e.g., about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, or about 100%), e.g., from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%, relative to a reference expression level (e.g., the expression level of the one or more genes set forth in Table 1 in a reference population, e.g., a median expression level of the one or more genes set forth in Table 1 in a reference population). In other instances, the expression level of the one or more genes set forth in Table 1 has been determined to be increased by about 0.5-fold, about 0.6-fold, about 0.7-fold, about 0.8-fold, about 0.9-fold, about 1-fold, about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, or about 10-fold or greater, e.g., from about 0.5-fold to about 0.7-fold, from about 0.7-fold to about 1-fold, from about 1-fold to about 1.5-fold, from about 1.5-fold to about 2-fold, from about 2-fold to about 3-fold, from about 3-fold to about 4-fold, from about 4-fold to about 5-fold, from about 5-fold to about 6-fold, from about 6-fold to about 7-fold, from about 7-fold to about 8-fold, or from about 9-fold to about 10-fold or greater, relative to the reference expression level (e.g., e.g., the expression level of the one or more genes set forth in Table 1 in a reference population, e.g., a median expression level of the one or more genes set forth in Table 1 in a reference population). In some embodiments, elevated or increased expression refers to an overall increase of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art-known methods such as those described herein, as compared to a reference level, reference sample, reference cell, reference tissue, control sample, control cell, or control tissue.

In certain embodiments, the elevated or increased expression refers to the increase in expression level (amount) of a biomarker in the sample, wherein the increase is at least about any of 1.5×, 1.75×, 2×, 3×, 4×, 5×, 6×, 7×, 8×, 9×, 10×, 25×, 50×, 75×, or 100× the expression level (amount) of the respective biomarker in a reference level, reference sample, reference cell, reference tissue, control sample, control cell, or control tissue. In some embodiments, elevated expression refers to an overall increase of greater than about 1.5-fold, about 1.75-fold, about 2-fold, about 2.25-fold, about 2.5-fold, about 2.75-fold, about 3.0-fold, or about 3.25-fold as compared to a reference sample, reference cell, reference tissue, control sample, control cell, control tissue, or internal control (e.g., housekeeping gene).

In certain embodiments, the method includes administering to a patient an IRAK4 pathway inhibitor when an increased expression level of one or more genes (e.g., 1, 2, 3, 4, 5, 6, 7, 8, 9, 10, 11, 12, 13, 14, 15, 16, 17, 18, 19, 20, 21, 22, 23, or all 24 genes) set forth in Table 1 relative to a reference expression level identifies the patient as having an increased likelihood of benefit from treatment with an IRAK4 pathway inhibitor.

In certain instances, the method includes administering to a patient having an IRAK4-mediated disorder or condition an IRAK4 pathway inhibitor when an increased level of expression of one or more genes (e.g., one gene, two genes, three genes, four genes, five genes, six genes, seven genes, eight genes, nine genes, ten genes, eleven genes, or all twelve genes) selected from the group consisting of CD38, SOCS3, AQP9, CDKN1A, GADD45B, B4GALT5, IL15RA, TNFAIP3, SOCS1, IL1RN, PFKFB3, and BCL2A1, relative to a reference expression level, identifies the patient as having an increased likelihood of benefit from treatment with an IRAK4 pathway inhibitor. In some instances, the method includes administering to a patient having an IRAK4-mediated disorder or condition an IRAK4 pathway inhibitor when an increased level of expression of one or more genes (e.g., one gene, two genes, three genes, four genes, five genes, six genes, seven genes, eight genes, nine genes, ten genes, or all eleven genes) selected from the group consisting of CD38, SOCS3, AQP9, CDKN1A, GADD45B, B4GALT5, IL15RA, TNFAIP3, SOCS1, IL1RN, and PFKFB3, relative to a reference expression level, identifies the patient as having an increased likelihood of benefit from treatment with an IRAK4 pathway inhibitor. In some instances, the method includes administering to a patient having an IRAK4-mediated disorder or condition an IRAK4 pathway inhibitor when an increased level of expression of one or more genes (e.g., one gene, two genes, three genes, four genes, five genes, six genes, seven genes, eight genes, or all nine genes) selected from the group consisting of CD38, SOCS3, AQP9, CDKN1A, GADD45B, B4GALT5, IL15RA, TNFAIP3, and SOCS1, relative to a reference expression level, identifies the patient as having an increased likelihood of benefit from treatment with an IRAK4 pathway inhibitor.

In some embodiments, the reference expression level can be: (i) the expression level of the one or more genes set forth in Table 1 in a reference population (e.g., a median expression level of the one or more genes set forth in Table 1 in a reference population); or (ii) a pre-assigned expression level for the one or more genes set forth in Table 1.

In any of the above methods, administration of an IRAK4 pathway inhibitor can have the therapeutic effect (i.e., benefit) of a cellular or biological response, a complete response, a partial response, a stable disease (without progression or relapse), or a response with a later relapse of the patient from, or as a result of, the treatment with the IRAK4 pathway inhibitor. Responsiveness to treatment with the IRAK4 pathway inhibitor will be evaluated and assessed by different means in accordance with standard medical practice for the specific IRAK4-mediated disorder or condition. Generally, the physician of skill will look for reduction in the signs and symptoms of the specific disease. The following are by way of examples.

For the IRAK4-mediated disorders of lupus and SLE, SLEDAI scores provide a numerical quantitation of disease activity. The SLEDAI is a weighted index of 24 clinical and laboratory parameters known to correlate with disease activity, with a numerical range of 0-103. see Bryan Gescuk & John Davis, “Novel therapeutic agent for systemic lupus erythematosus” in Current Opinion in Rheumatology 2002, 14:515-521. Antibodies to double-stranded DNA are believed to cause renal flares and other manifestations of lupus. Patients undergoing antibody treatment can be monitored for time to renal flare, which is defined as a significant, reproducible increase in serum creatinine, urine protein or blood in the urine. Alternatively or in addition, patients can be monitored for levels of antinuclear antibodies and antibodies to double-stranded DNA.

For the IRAK4-mediated disorder of rheumatoid arthritis (RA), measurements for progress in treatment may include the number of swollen and tender joints and the length of morning stiffness. Patients may be examined for how much the joint in the hands and feet have eroded by using X-rays and a scoring system known as the Sharp score. Another scoring system is based on the American College of Rheumatology criteria for assessing response to therapies. One method of evaluating treatment efficacy in RA is based on American College of Rheumatology (ACR) criteria, which measures the percentage of improvement in tender and swollen joints, among other things. The RA patient can be scored at for example, ACR 20 (20 percent improvement) compared with no antibody treatment (e.g., baseline before treatment) or treatment with placebo. Other ways of evaluating the efficacy of antibody treatment include X-ray scoring such as the Sharp X-ray score used to score structural damage such as bone erosion and joint space narrowing. Patients can also be evaluated for the prevention of or improvement in disability based on Health Assessment Questionnaire [HAQ] score, AIMS score, SF-36 at time periods during or after treatment. The ACR 20 criteria may include 20% improvement in both tender (painful) joint count and swollen joint count plus a 20% improvement in at least 3 of 5 additional measures:

1. patient's pain assessment by visual analog scale (VAS),

2. patient's global assessment of disease activity (VAS),

3. physician's global assessment of disease activity (VAS),

4. patient's self-assessed disability measured by the Health Assessment Questionnaire, and

5. acute phase reactants, CRP or ESR.

The ACR 50 and 70 are defined analogously. Preferably, the patient is administered an amount of a CD20 binding antibody of the invention effective to achieve at least a score of ACR 20, preferably at least ACR 30, more preferably at least ACR50, even more preferably at least ACR70, most preferably at least ACR 75 and higher.

In some instances, administration of an IRAK4 pathway inhibitor has the therapeutic effect of reducing or delaying progression of the IRAK4-mediated disorder or condition by 1 day or more (e.g., by 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, or 1 year or more) compared to treatment that does not include an IRAK4 pathway inhibitor.

In any of the preceding therapeutic methods, the IRAK4-mediated disorder or condition is selected from the group consisting of an immune disorder, an inflammatory disorder, a fibrotic disorder, an eosinophilic disorder, an infection, pain, a central nervous system disorder, an acute kidney injury, a chronic kidney disease, endometriosis, non-alcoholic fatty liver disease, a metabolic syndrome, and obesity.

In some instances, the immune disorder is allergic airway syndrome, allergic rhinitis, allograft rejection, asthma, atopic dermatitis, contact dermatitis, Crohn's disease, cutaneous lupus, delayed hypersensitivity, diabetes, gout, graft versus host disease, graft rejection, inflammatory bowel disease (IBD), inflammatory myositis (e.g., polymyositis, dermatomyositis), lupus, lupus nephritis, multiple sclerosis, psoriasis, rheumatoid arthritis, scleroderma, sepsis, systemic lupus erythematosus, systemic sclerosis, or ulcerative colitis.

In some instances, the inflammatory disorder is acute respiratory distress syndrome, acute lung injury, adult onset Still's disease, allergic airway syndrome, allergic rhinitis, asthma, atherosclerosis, atopic dermatitis, bronchitis, calcium pyrophosphate deposition disease (CPPD), cerebrovascular accident (e.g., stroke), chronic obstructive pulmonary disease (COPD), contact dermatitis, Crohn's disease, cryopyrin-associated periodic syndromes (CAPS), cutaneous lupus, delayed hypersensitivity, gout, graft versus host disease, inflammatory bowel disease (IBD), inflammatory myositis (e.g., polymyositis, dermatomyositis), lupus, lupus nephritis, rheumatoid arthritis, rhinitis, scleroderma, sepsis, systemic lupus erythematosus, systemic onset juvenile idiopathic arthritis, systemic sclerosis, or ulcerative colitis.

In some instances, the eosinophilic disorder is allergic rhinitis, asthma, atopic dermatitis, chronic obstructive pulmonary disease (COPD), or contact dermatitis.

In some instances, the fibrotic disorder is atherosclerosis, scleroderma, or systemic sclerosis.

In some instances, the central nervous system disorder is cerebrovascular accident (e.g., stroke), multiple sclerosis, or neurodegeneration.

In some instances, the pain is neuropathic pain.

In some instances, the infection is bronchitis or sepsis.

As described above, the IRAK4 pathway inhibitor may be an IRAK4 inhibitor, an IRAK1 inhibitor, a toll-like receptor (TLR) inhibitor, an interleukin-1 receptor (IL-1R) inhibitor, an interleukin-33 receptor (IL-33R) inhibitor, or a myeloid differentiation primary response gene 88 (MyD88) inhibitor. In one instance, the IRAK4 pathway inhibitor is an IRAK4 inhibitor. In some instances, the IRAK4 pathway inhibitor is a TLR inhibitor. In some instances, the TLR inhibitor is a TLR7 inhibitor, a TLR8 inhibitor, a TLR9 inhibitor, a TLR1 inhibitor, a TLR2 inhibitor, a TLR4 inhibitor, a TLRS inhibitor, a TLR6 inhibitor, ora TLR10 inhibitor. In certain instances, the IRAK4 pathway inhibitor is a TLR7 inhibitor, a TLR8 inhibitor, or both a TLR7 and TLR8 inhibitor. In certain instances, the IRAK4 pathway inhibitor is a TLR9 inhibitor. In any one of the preceding embodiments, the IRAK4 pathway inhibitor is a small molecule inhibitor. In other embodiments, the IRAK4 pathway inhibitor is a protein or multi-protein complex, such as an antibody.

Dosage and Administration

Once a patient responsive or sensitive to treatment with an IRAK4 pathway inhibitor has been identified, treatment with the IRAK4 pathway inhibitor, alone or in combination with an additional therapeutic agent, can be carried out. Such treatment may result in, for example, reducing or delaying progression of the IRAK4-mediated disorder or condition by 1 day or more (e.g., by 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, or 1 year or more) compared to treatment that does not include an IRAK4 pathway inhibitor. Moreover, treatment with the combination of an IRAK4 pathway inhibitor and at least one additional therapeutic agent preferably results in an additive, more preferably synergistic (or greater than additive), therapeutic benefit to the patient. Preferably, in this combination method the timing between at least one administration of the IRAK4 pathway inhibitor and at least one additional therapeutic agent is about one month or less, and more preferably, about two weeks or less.

It will be appreciated by those of skill in the art that the exact manner of administering a therapeutically effective amount of an IRAK4 pathway inhibitor to a patient following diagnosis of their likely responsiveness to the IRAK4 pathway inhibitor will be at the discretion of the attending physician. The mode of administration, including dosage, combination with other agents, timing and frequency of administration, and the like, may be affected by the diagnosis of a patient's likely responsiveness to such IRAK4 pathway inhibitor, as well as the patient's condition and history. Thus, even patients having IRAK4-mediated disorders or conditions (e.g., immune disorders (e.g., SLE) or inflammatory disorders (e.g., asthma)) who are predicted to be relatively insensitive to an IRAK4 pathway inhibitor may still benefit from treatment therewith, particularly in combination with other agents, including agents that may alter a patient's responsiveness to the antagonist.

A composition comprising an IRAK4 pathway inhibitor will be formulated, dosed, and administered in a fashion consistent with good medical practice. Factors for consideration in this context include the particular type of IRAK4-mediated disorder or condition being treated (e.g., immune disorder, inflammatory disorder, fibrotic disorder, eosinophilic disorder, infection, pain, central nervous system disorder, acute kidney injury, chronic kidney disease, endometriosis, non-alcoholic fatty liver disease, metabolic syndrome, and obesity), the particular mammal being treated (e.g., human), the clinical condition of the individual patient, the cause of the IRAK4-mediated disorder or condition, the site of delivery of the agent, possible side-effects, the type of inhibitor, the method of administration, the scheduling of administration, and other factors known to medical practitioners. The effective amount of the IRAK4 pathway inhibitor to be administered will be governed by such considerations.

A physician having ordinary skill in the art can readily determine and prescribe the effective amount of the pharmaceutical composition required, depending on such factors as the particular type of IRAK4 pathway inhibitor used. For example, the physician could start with doses of such an IRAK4 pathway inhibitor, employed in the pharmaceutical composition at levels lower than that required in order to achieve the desired therapeutic effect and gradually increase the dosage until the desired effect is achieved. The effectiveness of a given dose or treatment regimen of the inhibitor can be determined, for example, by assessing signs and symptoms in the patient using standard measures of efficacy.

In certain examples, the IRAK4 pathway inhibitor may be the only agent administered to the subject (i.e., as a monotherapy).

In certain examples, the patient is treated with the same IRAK4 pathway inhibitor at least twice. Thus, the first and second doses of the IRAK4 pathway inhibitor are preferably with the same IRAK4 pathway inhibitor (or at least the same class/type of IRAK4 pathway inhibitor, e.g., doses with the same or different IRAK4 inhibitor), and more preferably all doses of the IRAK4 pathway inhibitor are with the same IRAK4 pathway inhibitor, i.e., treatment for the first two doses, and preferably all doses, is with one class/type of IRAK4 pathway inhibitor (e.g., all doses with the same or different IRAK4 inhibitor).

Treatment with IRAK4 pathway inhibitors, or pharmaceutically acceptable salts thereof, can be carried out according to standard methods.

If multiple doses of an IRAK4 pathway inhibitor are provided, each dose may be provided using the same or a different administration means. In one embodiment, each dose is given by oral administration. In one embodiment, each dose is by intravenous administration. In another embodiment, each dose is given by subcutaneous administration. In yet another embodiment, the doses are given by both intravenous and subcutaneous administration.

The duration of therapy can be continued for as long as medically indicated or until a desired therapeutic effect (e.g., those described herein) is achieved. In certain embodiments, the therapy is continued for 1 month, 2 months, 4 months, 6 months, 8 months, 10 months, 1 year, 2 years, 3 years, 4 years, 5 years, or fora period of years up to the lifetime of the subject.

As noted above, however, these suggested amounts of IRAK4 pathway inhibitors are subject to a great deal of therapeutic discretion. The key factor in selecting an appropriate dose and scheduling is the result obtained, as indicated above. In some embodiments, the IRAK4 pathway inhibitor is administered as close to the first sign, diagnosis, appearance, or occurrence of the IRAK4-mediated disorder or condition being treated (e.g., immune disorder, inflammatory disorder, fibrotic disorder, eosinophilic disorder, infection, pain, central nervous system disorder, acute kidney injury, chronic kidney disease, endometriosis, non-alcoholic fatty liver disease, metabolic syndrome, and obesity) as possible.

1. Routes of Administration

IRAK4 pathway inhibitors and any additional therapeutic agents may be formulated, dosed, and administered in a fashion consistent with good medical practice. Factors for consideration in this context include the particular IRAK4-mediated disorder or condition being treated (e.g., immune disorder, inflammatory disorder, fibrotic disorder, eosinophilic disorder, infection, pain, central nervous system disorder, acute kidney injury, chronic kidney disease, endometriosis, non-alcoholic fatty liver disease, metabolic syndrome, and obesity), the clinical condition of the individual patient, the cause of the disorder, the site of delivery of the agent, the method of administration, the scheduling of administration, and other factors known to medical practitioners. The IRAK4 pathway inhibitor need not be, but is optionally formulated with and/or administered concurrently with, one or more agents currently used to prevent or treat the IRAK4-mediated disorder or condition being treated (e.g., immune disorder, inflammatory disorder, fibrotic disorder, eosinophilic disorder, infection, pain, central nervous system disorder, acute kidney injury, chronic kidney disease, endometriosis, non-alcoholic fatty liver disease, metabolic syndrome, and obesity).

For the prevention or treatment of an IRAK4-mediated disorder or condition being treated (e.g., immune disorder, inflammatory disorder, fibrotic disorder, eosinophilic disorder, infection, pain, central nervous system disorder, acute kidney injury, chronic kidney disease, endometriosis, non-alcoholic fatty liver disease, metabolic syndrome, and obesity), the appropriate dosage of an IRAK4 pathway inhibitor described herein (when used alone or in combination with one or more other additional therapeutic agents) will depend on the type of disease to be treated, the severity and course of the disease, whether the IRAK4 pathway inhibitor is administered for preventive or therapeutic purposes, previous therapy, the patient's clinical history and response to the IRAK4 pathway inhibitor, and the discretion of the attending physician. The IRAK4 pathway inhibitor is suitably administered to the patient at one time or over a series of treatments. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the IRAK4 pathway inhibitor). An initial higher loading dose, followed by one or more lower doses may be administered. However, other dosage regimens may be useful. The progress of this therapy is easily monitored by conventional techniques and assays.

The IRAK4 pathway inhibitor can be administered by any suitable means, including orally, parenteral, topical, subcutaneous, intraperitoneal, intrapulmonary, intranasal, and/or intralesional administration. Parenteral infusions include intramuscular, intravenous, intraarterial, intraperitoneal, or subcutaneous administration. Intrathecal administration is also contemplated. In addition, the IRAK4 pathway inhibitor may suitably be administered by pulse infusion, e.g., with declining doses of the IRAK4 pathway inhibitor.

If multiple doses of an IRAK4 pathway inhibitor are provided, each dose may be provided using the same or a different administration means. In one embodiment, each dose is by oral administration. For example, one or more IRAK4 pathway inhibitors can provided in tablet form. For example, one or more IRAK4 pathway inhibitors can be administered twice a day. In another embodiment, each exposure is given intravenously (i.v.). In another embodiment, each exposure is given by subcutaneous (s.c.) administration. In yet another embodiment, the exposures are given by both i.v. and s.c. administration.

2. Combination Therapy

The methods may further involve administering to the patient an effective amount of an IRAK4 pathway inhibitor in combination with an additional therapeutic agent. In some instances, the additional therapeutic agent is an additional IRAK4 pathway inhibitor. In some instances, the additional therapeutic agent is a corticosteroid, a nonsteroidal anti-inflammatory drug (NSAID), chloroquine, hydroxychloroquine (PLAQUENIL®), cyclosporine, azathioprine, methotrexate, mycophenolate mofetil (CELLCEPT®), or cyclophosphamide (CYTOXAN®). In some instances, the IRAK4 pathway inhibitor is used in combination with surgery.

The combination therapy may provide “synergy” and prove “synergistic,” i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially. In general, during alternation therapy, an effective dosage of each active ingredient is administered sequentially (i.e., serially), whereas in combination therapy, effective dosages of two or more active ingredients are administered together.

As described above, the therapeutic methods may include administering a combination of two or more (e.g., three or more) IRAK4 pathway inhibitors. In some instances, for example, two IRAK4 inhibitors are administered in combination, either sequentially or concomitantly. In some instances, for example, two IRAK1 inhibitors are administered in combination, either sequentially or concomitantly. In some instances, for example, two TLR inhibitors are administered in combination, either sequentially or concomitantly. In some instances, for example, two IL-1R inhibitors are administered in combination, either sequentially or concomitantly. In some instances, for example, two IL-33R inhibitors are administered in combination, either sequentially or concomitantly. In some instances, for example, two MyD88 inhibitors are administered in combination, either sequentially or concomitantly.

In general, for the prevention or treatment of disease, the appropriate dosage of the additional therapeutic agent will depend on the type of disease to be treated, the type of antibody, the severity and course of the disease, whether the IRAK4 pathway inhibitor and additional agent (e.g., a corticosteroid) are administered for preventive or therapeutic purposes, previous therapy, the patient's clinical history and response to the IRAK4 pathway inhibitor and additional agent, and the discretion of the attending physician. The IRAK4 pathway inhibitor and additional agent are suitably administered to the patient at one time or over a series of treatments. The IRAK4 pathway inhibitor is typically administered as set forth above. Depending on the type and severity of the disease, about 20 mg/m² to 600 mg/m² of the additional agent is an initial candidate dosage for administration to the patient, whether, for example, by one or more separate administrations, or by continuous infusion. One typical daily dosage might range from about or about 20 mg/m², 85 mg/m², 90 mg/m², 125 mg/m², 200 mg/m², 400 mg/m², 500 mg/m² or more, depending on the factors mentioned above. For repeated administrations over several days or longer, depending on the condition, the treatment is sustained until a desired suppression of disease symptoms occurs. Thus, one or more doses of about 20 mg/m², 85 mg/m², 90 mg/m², 125 mg/m², 200 mg/m², 400 mg/m², 500 mg/m², 600 mg/m² (or any combination thereof) may be administered to the patient. Such doses may be administered intermittently, e.g., every week or every two, three weeks, four, five, or six (e.g., such that the patient receives from about two to about twenty, e.g., about six doses of the additional agent). An initial higher loading dose, followed by one or more lower doses may be administered. However, other dosage regimens may be useful. The progress of this therapy is easily monitored by conventional techniques and assays.

In one embodiment, the patient has never been previously administered any drug(s) to treat an IRAK4-mediated disorder or condition. In another embodiment, the patient have been previously administered one or more medicaments(s) to treat an IRAK4-mediated disorder or condition. In a further embodiment, the patient was not responsive to one or more of the medicaments that had been previously administered to treat an IRAK4-mediated disorder or condition. Such drugs to which the subject may be non-responsive include, for example, one or more of a corticosteroid, a nonsteroidal anti-inflammatory drug (NSAID), chloroquine, hydroxychloroquine (PLAQUENIL®), cyclosporine, azathioprine, methotrexate, mycophenolate mofetil (CELLCEPT®), and/or cyclophosphamide (CYTOXAN®), not administered in combination with an IRAK4 pathway inhibitor.

### VI. Diagnostic Kits and Compositions

The invention further provides diagnostic kits and compositions that include one or more reagents (e.g., polypeptides [such as antibodies or antigen-binding fragments thereof] or polynucleotides [such as probes or primers]) for determining the expression level of one or more genes (e.g., 1, 2, 3, 4, 5, 6, 7, 8, 9, 10, 11, 12, 13, 14 , 15, 16, 17, 18, 19, 20, 21, 22, 23, or all 24 genes) set forth in Table 1 in a sample from an individual or patient having an IRAK4-mediated disorder or condition (e.g., immune disorder, inflammatory disorder, fibrotic disorder, eosinophilic disorder, infection, pain, central nervous system disorder, acute kidney injury, chronic kidney disease, endometriosis, non-alcoholic fatty liver disease, metabolic syndrome, and obesity). In some instances, an increased expression level of the one or more genes set forth in Table 1, relative to a reference expression level, identifies a patient having an IRAK4-mediated disorder or condition who may benefit from treatment comprising the IRAK4 pathway inhibitor. In other instances, a decreased expression level of the one or more genes set forth in Table 1, relative to a reference expression level, identifies a patient having an IRAK4-mediated disorder or condition who may benefit from treatment comprising the IRAK4 pathway inhibitor. Optionally, the kit may further include instructions to use the kit to identify a patient with a higher likelihood of benefiting from treatment with an IRAK4 pathway inhibitor. In another instance, the kit may further include instructions to use the kit to select a medicament (e.g., a medicament including an IRAK4 pathway inhibitor, such as an IRAK4 inhibitor, an IRAK1 inhibitor, a toll-like receptor (TLR) inhibitor, an interleukin-1 receptor (IL-1 R) inhibitor, an interleukin-33 receptor (IL-33R) inhibitor, or a myeloid differentiation primary response gene 88 (MyD88) inhibitor, or combinations thereof) for treating a patient having an IRAK4-mediated disorder or condition if the patient is treatment naïve and has an increased expression level of the one or more genes set forth in Table 1, relative to a reference expression level. In another instance, the kit may further include instructions to use the kit to select a medicament (e.g., a medicament including an IRAK4 pathway inhibitor, such as an IRAK4 inhibitor, an IRAK1 inhibitor, a toll-like receptor (TLR) inhibitor, an interleukin-1 receptor (IL-1 R) inhibitor, an interleukin-33 receptor (IL-33R) inhibitor, or a myeloid differentiation primary response gene 88 (MyD88) inhibitor, or combinations thereof) for treating a patient having an IRAK4-mediated disorder or condition if the patient has a decreased expression level of the one or more genes set forth in Table 1, relative to a reference expression level, after receiving a first dose of a treatment including an IRAK4 pathway inhibitor.

The compositions of the invention include polypeptides (e.g., antibodies or antigen-binding fragments thereof) or polynucleotides (e.g., probes and/or primers) capable of determining the expression level of RNASE4 and, optionally one or more other biomarkers (e.g., PSA and/or ANG).

### EXAMPLES

The following examples are provided to illustrate, but not to limit, the presently claimed invention.

### Example 1. Identification of Genes with Impaired Response to TLR7/8 Stimulation in IRAK4-Deficient Patients

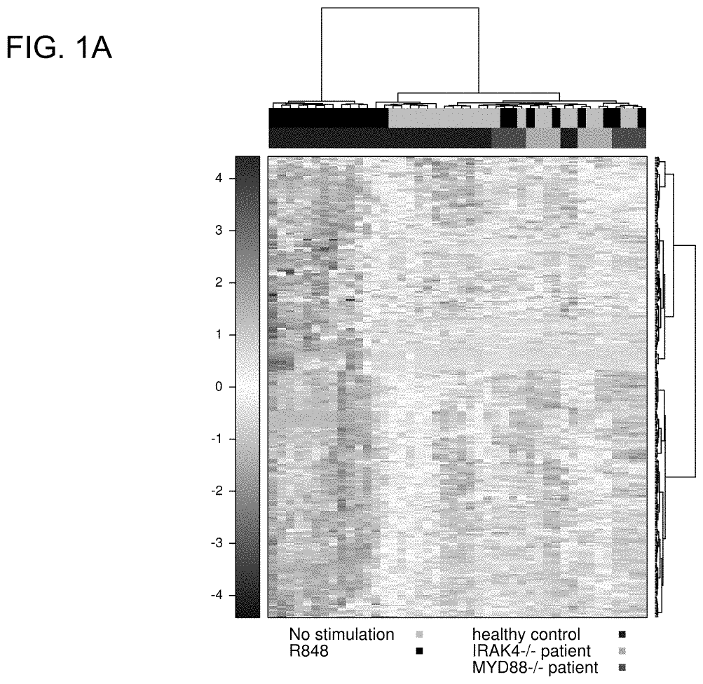

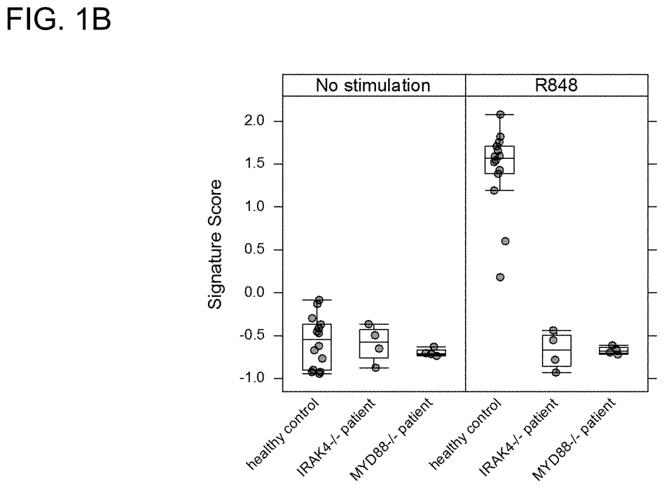

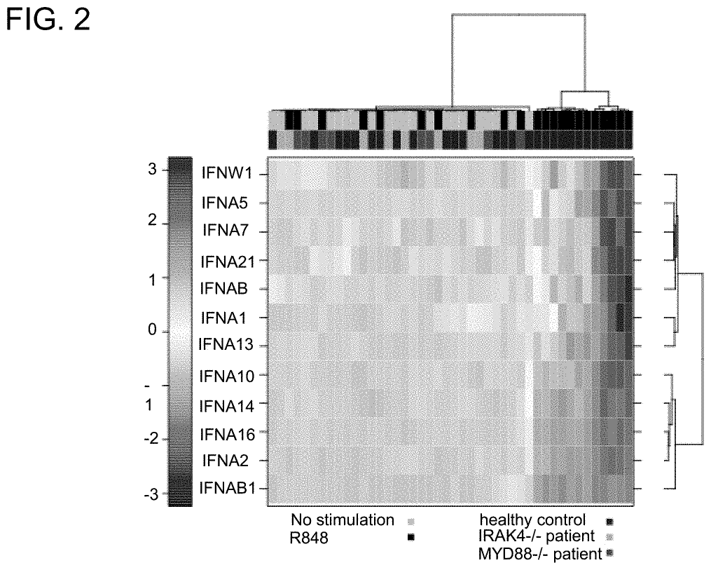

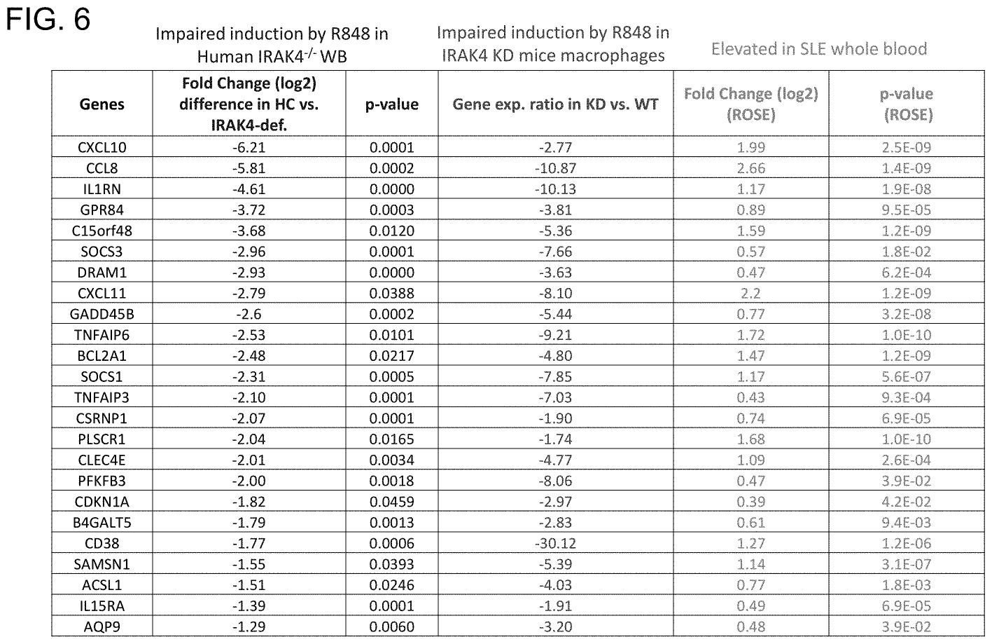

To determine candidate IRAK4 biomarker genes that can serve as both pharmacodynamic and predictive biomarkers, genes were first identified that displayed significantly lower induction following TLR stimulation in patients carrying loss-of-function IRAK4 mutations compared to healthy patients. To this end, an analysis of the microarray dataset of GEO Accession GSE25742 (Alsina et al. Nat. lmmunol. 15:1134-42, 2014), a genome-wide microarray expression profiling study of whole blood from patients with defects in toll-like receptor (TLR) and interleukin-1 receptor (IL-1R) (i.e., the Toll/IL-1 receptor (TIR) pathway) signaling, was performed. Analysis of the microarray dataset identified 285 genes that displayed significantly lower induction by the TLR7/8 stimulator R848 (resiquimod) in the whole blood from IRAK4-deficient patients compared to healthy patient controls (false discovery rate (FDR)<0.05; fold-change (FC)>1.25) (FIGS. 1A-1B and 6). Whereas the healthy patient controls were found to upregulate type I interferons (IFNs) in response to R848, IRAK4⁺ patients failed to upregulate type I IFNs and other TLR-regulated genes in response to R848 (FIG. 2).

### Example 2. Identification of Genes Upregulated in Systemic Lupus Erythematosus (SLE) Patients

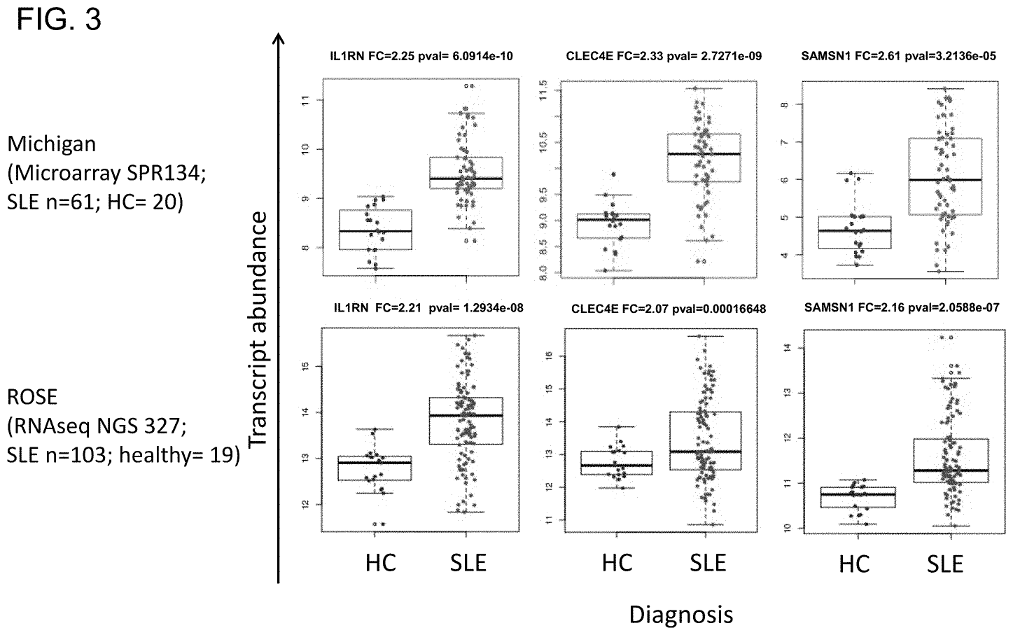

Analysis of two extra-renal systemic lupus erythematosus (SLE) patient cohorts was performed to determine which of the 285 differentially expressed genes identified in Example 1 also showed elevated baseline expression in SLE patients. Peripheral blood mononuclear cell (PBMC) microarray data (University of Michigan Cohort; SLE (n=61), healthy controls (HC) (n=20)) and whole blood RNA sequencing data (ROSE Phase II Study (Kalunian et al. Ann. Rheum. Dis. 75: 196-202, 2016); SLE (n=103), HC (n=19)) were analyzed for differential expression between the SLE and HC groups in both datasets. 44 genes of the 285 differentially expressed genes were subsequently identified as upregulated in SLE patients compared to healthy patients in both datasets (p<0.05; FC>1.2) (FIGS. 3 and 6).

### Example 3. Characterization of IRAK4 Pathway Genes Using IRAK4 Kinase-Dead Mice

IRAK4 kinase-dead (KD) mice were generated to further characterize the putative IRAK4 pathway genes identified in Example 2. Genomic DNA for IRAK4 was isolated from a 129/J mouse genomic library using the 5′ end of the mouse IRAK4 complementary DNA as a probe. Using standard cloning techniques, the targeting construct was designed to disrupt the ATG start codon and to replace part of exon 2 of the IRAK4 gene with a PGK-Neo cassette. The targeting plasmid was linearized and transfected into embryonic stem cells of a 129/Ola background (E14 clone). Homologously recombined embryonic stem cell lines were injected into day 3.5 C57BL/6J (B6) blastocysts that were subsequently transferred to CD1 pseudo-pregnant foster mothers to generate chimeric progeny. Male chimeras were back-crossed to C57BL/6J females to produce agouti progeny. Germline transmission in F1 heterozygous offspring was verified by Southern blot analysis, and the F1 heterozygotes were interbred to obtain homozygous mutant mice.

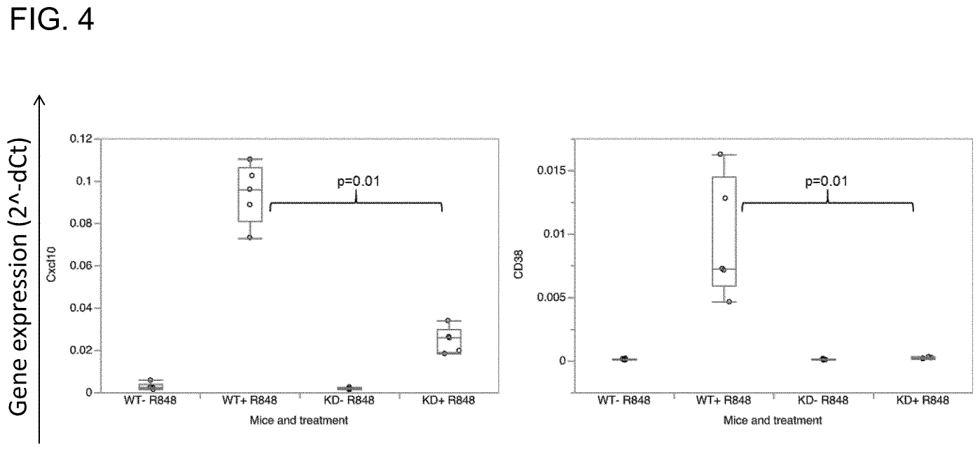

Bone marrow-derived macrophages (BMDMs) from the IRAK4 KD mice (n=5) and wild-type control mice (n=5) were harvested and stimulated in vitro for four hours with R848, a TLR7 agonist. Gene expression analyses were performed by Fluidigm for the putative IRAK4 pathway biomarker genes in unstimulated and stimulated BMDMs from IRAK4 KD and wild-type control mice. The gene expression analyses showed that the putative IRAK4 pathway biomarker genes displayed impaired induction by TLR7 in BMDMs from IRAK4 KD mice compared to IRAK4 wild-type mice (FIGS. 4 and 6). Similar gene expression analyses of whole blood from human IRAK4^{-/-} patients showed that certain putative IRAK4 pathway biomarker genes also displayed impaired induction by TLR7 (FIG. 6). In particular, 24 genes of the 44 total putative IRAK pathway biomarker genes were found to display impaired induction by TLR7 in both human and mouse. These 24 IRAK4 pathway biomarkers are listed in Table 1 and described herein and highlight the commonality between human and mouse systems.

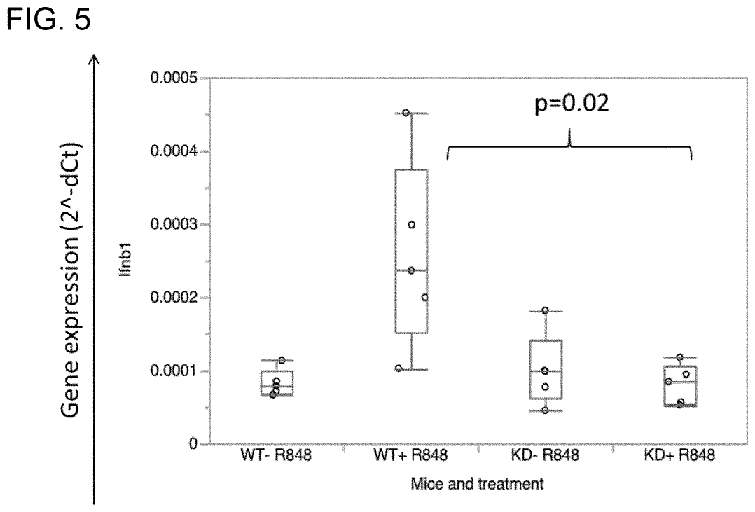

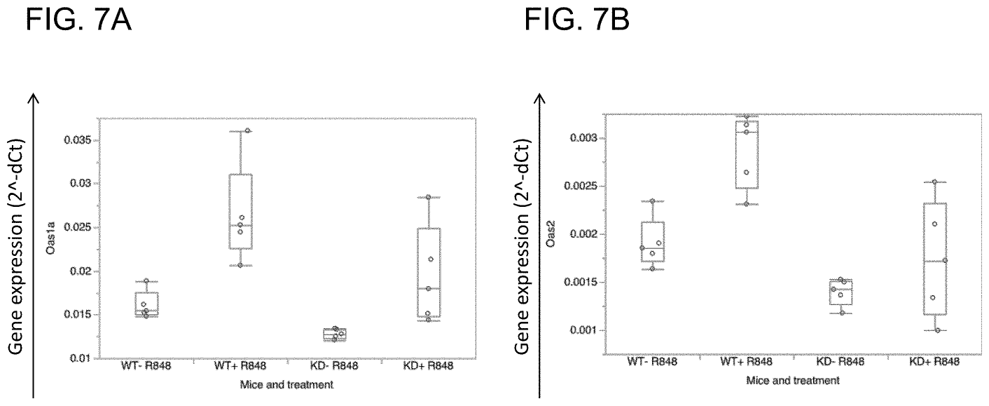

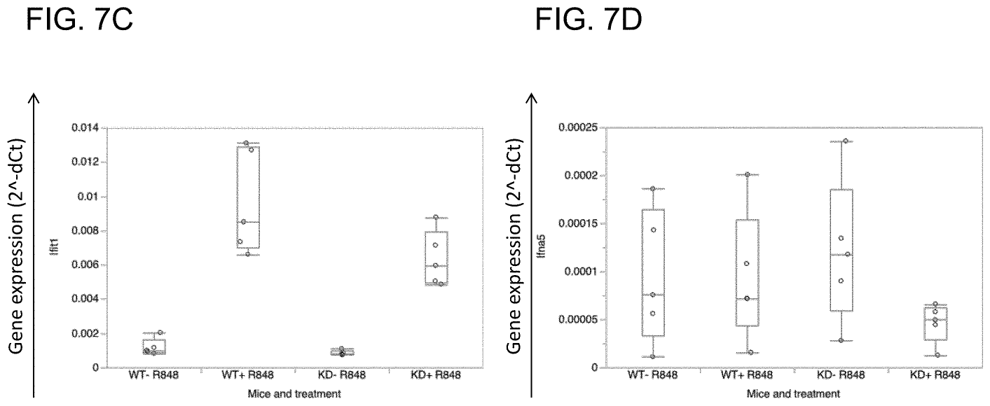

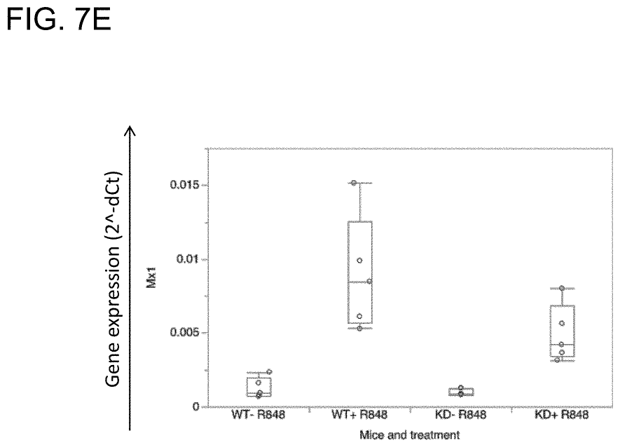

Additional gene expression analyses demonstrated that IFNβ1 displayed a lower induction by R848 in IRAK4 KD mice compared to IRAK4 wild-type mice (p=0.02) (FIG. 5). Other IFN-regulated genes (OAS1A, OAS2, IFIT1, IFNA5, and MX1) showed the same trend of decreased induction by R848 in IRAK4 KD mice compared to IRAK4 wild-type mice (p<0.15) (FIGS. 7A-7E).

### Example 4. Dose-Dependent Downregulation of IRAK4 Pathway Biomarker Genes by IRAK4 Small Molecule Inhibitors

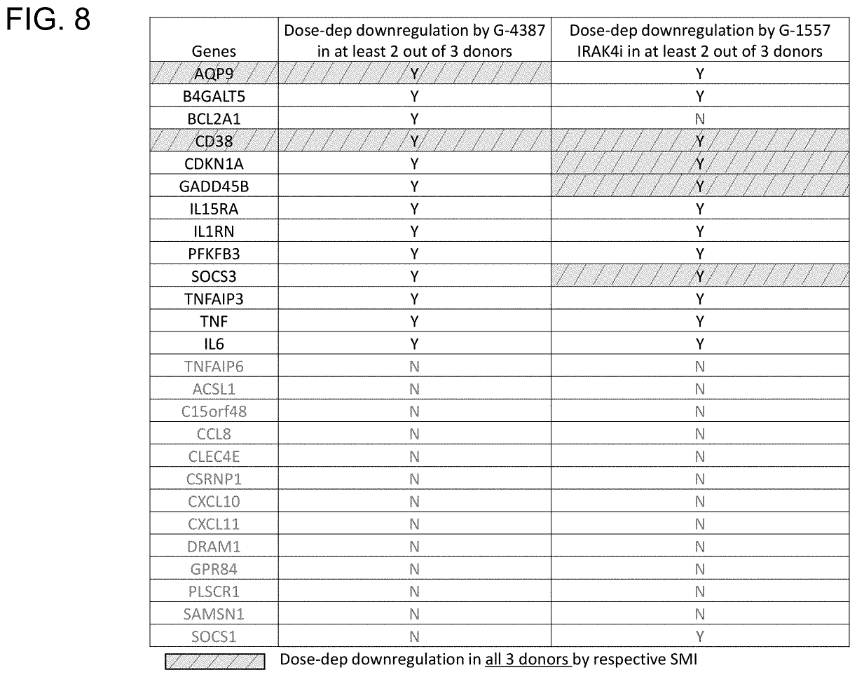

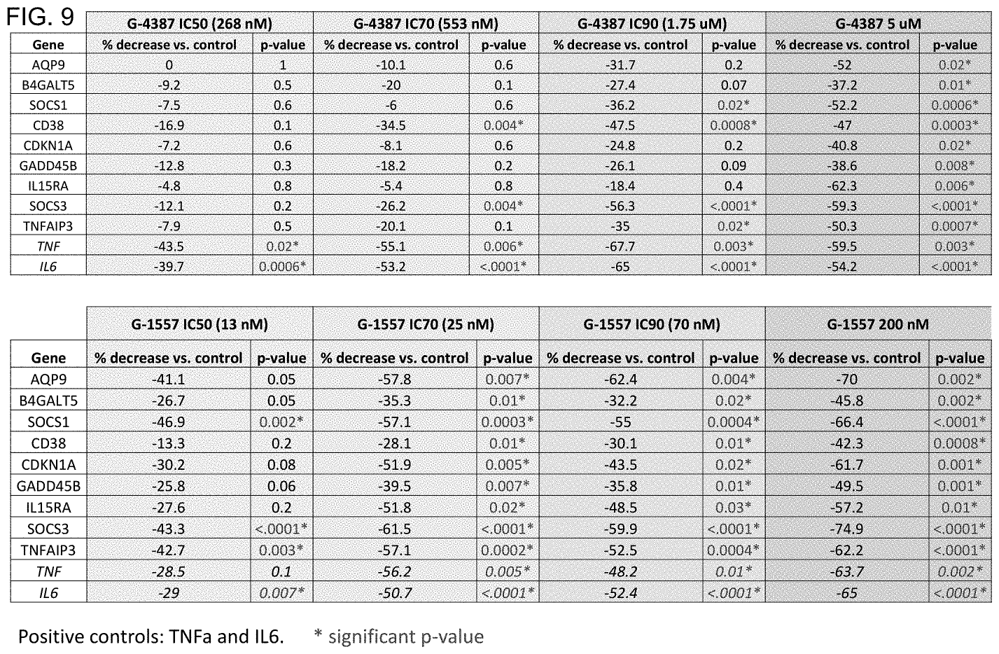

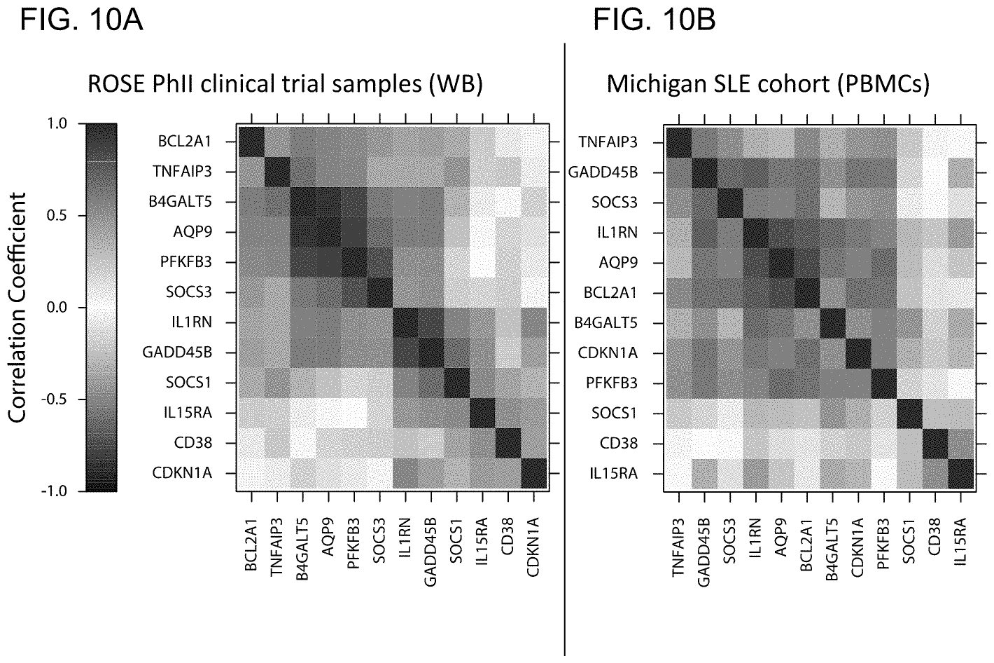

The 24 IRAK4 pathway biomarker genes were subsequently characterized based on inducibility by R848 and dose-dependent downregulation by two distinct IRAK4 small molecule inhibitor test compounds, G03074387 (G-4387) (BMS) and G03081557 (G-1557) (Pfizer), in human whole blood samples. Whole blood samples from three healthy donors were divided into six arms: (1) unstimulated; (2) stimulated with R848 at 1.25 μM (˜440 ng/ml); (3) G-4387 at IC50 (268 nM) for 1 hour+R848 stimulation for 3.5 hours; (4) G-4387 at IC70 (553 nM) for 1 hour+R848 stimulation for 3.5 hours; (5) G-4387 at IC90 (1.75 μM) for 1 hour+R848 stimulation for 3.5 hours; and (6) G-4387 at 5 μM for 1 hour+R848 stimulation for 3.5 hours. Similar experiments were conducted using whole blood samples from the same three healthy donors for G-1557, except that the dose of G-1557 in Arms (3)-(6) were as follows: (3) G-1557 at IC50 (13 nM); (4) G-1557 at IC70 (25 nM); (5) G-1557 at IC90 (70 nM); and (6) G-1557 at 200 nM. For both experiments, RNA was subsequently extracted and transcript levels of the 24 IRAK4 pathway biomarker genes were measured by qPCR. The results of these experiments are shown in FIGS. 8 and 9. Of the 24 IRAK4 pathway biomarker genes, nine genes (CD38, SOCS3, AQP9, CDKN1A, GADD45B, B4GALT5, IL15RA, TNFAIP3, and SOCS1) were dose-dependently downregulated by both G-4387 and G-1557 in whole blood (FIG. 9). In addition, an inter-correlated signature in SLE patient blood was observed for these IRAK4 biomarker genes in both ROSE Phase II and University of Michigan SLE Cohorts described above in Example 2 (FIGS. 10A-10B). Significant positive correlations were also observed between IRAK4 pathway biomarker genes and interferon signature metric (ISM) and anti-dsDNA status, as well as levels of BAFF, anti-RNP antibodies, and anti-Sm antibodies. Significant negative correlations were observed between the IRAK4 biomarker genes and levels of complement component 3 (C3) and complement component 4 (C4) in both SLE datasets.

Together, the data suggest that the coordinated expression of these IRAK4-regulated genes is reflective of TLR and other upstream stimulation and therefore reflects IRAK pathway activity, thereby supporting the utility of the identified IRAK4 pathway biomarker genes as pharmacodynamics and predictive diagnostic biomarkers for patients who are likely to respond to treatment including an IRAK4 pathway inhibitor (e.g., an IRAK4 small molecule inhibitor).

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of monitoring the response of a patient having an interleukin-1 receptor-associated kinase 4 (IRAK4)-mediated disorder or condition to treatment comprising an IRAK4 pathway inhibitor, the method comprising:
(a) determining, in a sample obtained from the patient at a time point following administration of a first dose of the IRAK4 pathway inhibitor, the expression level of one or more genes set forth in Table 1; and
(b) comparing the expression level of the one or more genes set forth in Table 1 in the sample with a reference expression level, thereby monitoring the response of the patient to treatment comprising the IRAK4 pathway inhibitor.

2. The method of claim 1, wherein the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, PFKFB3, and BCL2A1.

3. The method of claim 2, wherein the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, and PFKFB3.

4. The method of claim 3, wherein the one or more genes set forth in Table 1 comprises one or more genes selected from the group consisting of CD38, SOCS3, AQP9, CDKN1A, GADD45B, B4GALT5, IL15RA, TNFAIP3, and SOCS1.

5. The method of claim 4, wherein the one or more genes set forth in Table 1 comprises all nine of CD38, SOCS3, AQP9, CDKN1A, GADD45B, B4GALT5, IL15RA, TNFAIP3, and SOCS1.

6. The method of claim 5, wherein the one or more genes set forth in Table 1 comprises all 11 of CD38, SOCS3, AQP9, CDKN1A, GADD45B, B4GALT5, IL15RA, TNFAIP3, SOCS1, IL1RN, and PFKFB3.

7. The method of claim 6, wherein the one or more genes set forth in Table 1 comprises all 12 of CD38, SOCS3, AQP9, CDKN1A, GADD45B, B4GALT5, IL15RA, TNFAIP3, SOCS1, IL1RN, PFKFB3, and BCL2A1.

8. The method of claim 7, wherein the one or more genes set forth in Table 1 are all 24 genes set forth in Table 1.

9. The method of any one of claims 1-8, wherein the expression level of the one or more genes set forth in Table 1 is decreased in the sample obtained from the patient relative to the reference expression level.

10. The method of claim 9, wherein the expression level of the one or more genes set forth in Table 1 is decreased at least about 0.5-fold relative to the reference expression level.

11. The method of claim 10, wherein the expression level of the one or more genes set forth in Table 1 is decreased at least about 1-fold relative to the reference expression level.

12. The method of claim 11, wherein the expression level of the one or more genes set forth in Table 1 is decreased at least about 2-fold relative to the reference expression level.

13. The method of claim 12, wherein the expression level of the one or more genes set forth in Table 1 is decreased at least about 3-fold relative to the reference expression level.

14. The method of claim 13, wherein the expression level of the one or more genes set forth in Table 1 is decreased at least about 4-fold relative to the reference expression level.

15. The method of claim 14, wherein the expression level of the one or more genes set forth in Table 1 is decreased at least about 5-fold relative to the reference expression level.

16. The method of claim 15, wherein the expression level of the one or more genes set forth in Table 1 is decreased at least about 10-fold relative to the reference expression level.

17. The method of any one of claims 9-16, wherein the decreased expression level of the one or more genes set forth in Table 1 indicates that the patient is responding to the IRAK4 pathway inhibitor.

18. The method of claim 17, further comprising administering at least a second dose of an IRAK4 pathway inhibitor to a patient whose expression level of the one or more genes set forth in Table 1 is decreased relative to the reference expression level.

19. A method of treating a patient having an IRAK4-mediated disorder or condition with an IRAK4 pathway inhibitor, the method comprising:
(a) determining, in a sample obtained from the patient at a time point following administration of a first dose of the IRAK4 pathway inhibitor, the expression level of one or more genes set forth in Table 1;
(b) comparing the expression level of the one or more genes set forth in Table 1 in the sample with a reference expression level; and
(c) administering at least a second dose of the IRAK4 pathway inhibitor to the patient based on a decreased expression level of the one or more genes set forth in Table 1 relative to the reference expression level.

20. The method of claim 19, wherein the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, PFKFB3, and BCL2A1.

21. The method of claim 20, wherein the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, and PFKFB3.

22. The method of claim 21, wherein the one or more genes set forth in Table 1 comprises one or more genes selected from the group consisting of CD38, SOCS3, AQP9, CDKN1A, GADD45B, B4GALT5, IL15RA, TNFAIP3, and SOCS1.

23. The method of claim 22, wherein the one or more genes set forth in Table 1 comprises all nine of CD38, SOCS3, AQP9, CDKN1A, GADD45B, B4GALT5, IL15RA, TNFAIP3, and SOCS1.

24. The method of claim 23, wherein the one or more genes set forth in Table 1 comprises all 11 of CD38, SOCS3, AQP9, CDKN1A, GADD45B, B4GALT5, IL15RA, TNFAIP3, SOCS1, IL1RN, and PFKFB3.

25. The method of claim 24, wherein the one or more genes set forth in Table 1 comprises all 12 of CD38, SOCS3, AQP9, CDKN1A, GADD45B, B4GALT5, IL15RA, TNFAIP3, SOCS1, IL1RN, PFKFB3, and BCL2A1.

26. The method of claim 25, wherein the one or more genes set forth in Table 1 are all 24 genes set forth in Table 1.

27. The method of any one of claims 19-26, wherein the expression level of the one or more genes set forth in Table 1 is decreased at least about 0.5-fold relative to the reference expression level.

28. The method of claim 27, wherein the expression level of the one or more genes set forth in Table 1 is decreased at least about 1-fold relative to the reference expression level.

29. The method of claim 28, wherein the expression level of the one or more genes set forth in Table 1 is decreased at least about 2-fold relative to the reference expression level.

30. The method of claim 29, wherein the expression level of the one or more genes set forth in Table 1 is decreased at least about 3-fold relative to the reference expression level.

31. The method of claim 30, wherein the expression level of the one or more genes set forth in Table 1 is decreased at least about 4-fold relative to the reference expression level.

32. The method of claim 31, wherein the expression level of the one or more genes set forth in Table 1 is decreased at least about 5-fold relative to the reference expression level.

33. The method of claim 32, wherein the expression level of the one or more genes set forth in Table 1 is decreased at least about 10-fold relative to the reference expression level.

34. The method of any one of claims 1-33, wherein the reference expression level is:
(i) the expression level of the one or more genes set forth in Table 1 in a sample from the patient obtained prior to administration of the first dose of the IRAK4 pathway inhibitor;
(ii) the expression level of the one or more genes set forth in Table 1 in a reference population;
(iii) a pre-assigned expression level for the one or more genes set forth in Table 1;
(iv) the expression level of the one or more genes set forth in Table 1 in a sample obtained from the patient at a previous time point, wherein the previous time point is following administration of the first dose of the IRAK4 pathway inhibitor; or
(v) the expression level of the one or more genes set forth in Table 1 in a sample obtained from the patient at a subsequent time point.

35. A method of identifying a patient having an IRAK4-mediated disorder or condition who may benefit from treatment comprising an IRAK4 pathway inhibitor, the method comprising determining an expression level of one or more genes set forth in Table 1 in a sample obtained from the patient, wherein an increased expression level of the one or more genes set forth in Table 1 in the sample as compared to a reference expression level identifies the patient as one who may benefit from treatment comprising an IRAK4 pathway inhibitor.

36. A method of selecting a therapy for a patient having an IRAK4-mediated disorder or condition, the method comprising determining an expression level of one or more genes set forth in Table 1 in a sample obtained from the patient, wherein an increased expression level of the one or more genes set forth in Table 1 in the sample as compared to a reference expression level identifies the patient as one who may benefit from treatment comprising an IRAK4 pathway inhibitor.

37. The method of claim 35 or 36, wherein the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, PFKFB3, and BCL2A1.

38. The method of claim 37, wherein the one or more genes set forth in Table 1 comprises one or more genes selected from the group consisting of CD38, SOCS3, AQP9, CDKN1A, GADD45B, B4GALT5, IL15RA, TNFAIP3, SOCS1, URN, and PFKFB3.

39. The method of claim 38, wherein the one or more genes set forth in Table 1 comprises one or more genes selected from the group consisting of CD38, SOCS3, AQP9, CDKN1A, GADD45B, B4GALT5, IL15RA, TNFAIP3, and SOCS1.

40. The method of claim 39, wherein the one or more genes set forth in Table 1 comprises all nine of CD38, SOCS3, AQP9, CDKN1A, GADD45B, B4GALT5, IL15RA, TNFAIP3, and SOCS1.

41. The method of claim 40, wherein the one or more genes set forth in Table 1 comprises all 11 of CD38, SOCS3, AQP9, CDKN1A, GADD45B, B4GALT5, IL15RA, TNFAIP3, SOCS1, IL1RN, and PFKFB3.

42. The method of claim 41, wherein the one or more genes set forth in Table 1 comprises all 12 of CD38, SOCS3, AQP9, CDKN1A, GADD45B, B4GALT5, IL15RA, TNFAIP3, SOCS1, IL1RN, PFKFB3, and BCL2A1.

43. The method of claim 42, wherein the one or more genes set forth in Table 1 are all 24 genes set forth in Table 1.

44. The method of any one of claims 35-43, wherein the expression level of the one or more genes set forth in Table 1 is increased in the sample obtained from the patient relative to the reference expression level.

45. The method of claim 44, wherein the expression level of the one or more genes set forth in Table 1 is increased at least about 0.5-fold relative to the reference expression level.

46. The method of claim 45, wherein the expression level of the one or more genes set forth in Table 1 is increased at least about 1-fold relative to the reference expression level.

47. The method of claim 46, wherein the expression level of the one or more genes set forth in Table 1 is increased at least about 2-fold relative to the reference expression level.

48. The method of claim 47, wherein the expression level of the one or more genes set forth in Table 1 is increased at least about 3-fold relative to the reference expression level.

49. The method of claim 48, wherein the expression level of the one or more genes set forth in Table 1 is increased at least about 4-fold relative to the reference expression level.

50. The method of claim 49, wherein the expression level of the one or more genes set forth in Table 1 is increased at least about 5-fold relative to the reference expression level.

51. The method of claim 50, wherein the expression level of the one or more genes set forth in Table 1 is increased at least about 10-fold relative to the reference expression level.

52. The method of any one of claims 35-51, wherein the patient has an increased expression level of the one or more genes set forth in Table 1 relative to the reference expression level and the method further comprises administering to the patient an IRAK4 pathway inhibitor.

53. A method of treating a patient having an IRAK4-mediated disorder or condition, the method comprising administering to the patient an IRAK4 pathway inhibitor, wherein prior to treatment the expression level of one or more genes set forth in Table 1 in a sample obtained from the patient has been determined to be increased relative to a reference expression level.

54. The method of claim 53, wherein the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, PFKFB3, and BCL2A1.

55. The method of claim 54, wherein the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, and PFKFB3.

56. The method of claim 55, wherein the one or more genes set forth in Table 1 comprises one or more genes selected from the group consisting of CD38, SOCS3, AQP9, CDKN1A, GADD45B, B4GALT5, IL15RA, TNFAIP3, and SOCS1.

57. The method of claim 56, wherein the one or more genes set forth in Table 1 comprises all nine of CD38, SOCS3, AQP9, CDKN1A, GADD45B, B4GALT5, IL15RA, TNFAIP3, and SOCS1.

58. The method of claim 57, wherein the one or more genes set forth in Table 1 comprises all 11 of CD38, SOCS3, AQP9, CDKN1A, GADD45B, B4GALT5, IL15RA, TNFAIP3, SOCS1, IL1RN, and PFKFB3.

59. The method of claim 58, wherein the one or more genes set forth in Table 1 comprises all 12 of CD38, SOCS3, AQP9, CDKN1A, GADD45B, B4GALT5, IL15RA, TNFAIP3, SOCS1, IL1RN, PFKFB3, and BCL2A1.

60. The method of claim 59, wherein the one or more genes set forth in Table 1 are all 24 genes set forth in Table 1.

61. The method of any one of claims 53-60, wherein the expression level of the one or more genes set forth in Table 1 have been determined to be increased at least about 0.5-fold relative to the reference expression level.

62. The method of claim 61, wherein the expression level of the one or more genes set forth in Table 1 have been determined to be increased at least about 1-fold relative to the reference expression level.

63. The method of claim 62, wherein the expression level of the one or more genes set forth in Table 1 have been determined to be increased at least about 2-fold relative to the reference expression level.

64. The method of claim 63, wherein the expression level of the one or more genes set forth in Table 1 have been determined to be increased at least about 3-fold relative to the reference expression level.

65. The method of claim 64, wherein the expression level of the one or more genes set forth in Table 1 have been determined to be increased at least about 4-fold relative to the reference expression level.

66. The method of claim 65, wherein the expression level of the one or more genes set forth in Table 1 have been determined to be increased at least about 5-fold relative to the reference expression level.

67. The method of claim 66, wherein the expression level of the one or more genes set forth in Table 1 have been determined to be increased at least about 10-fold relative to the reference expression level.

68. The method of any one of claims 35-67, wherein the reference expression level is:
(i) the expression level of the one or more genes set forth in Table 1 in a reference population; or
(ii) a pre-assigned expression level for the one or more genes set forth in Table 1.

69. The method of claim 34 or 68, wherein the expression level of the one or more genes set forth in Table 1 in a reference population is a median expression level of the one or more genes set forth in Table 1 in a reference population.

70. The method of any one of claims 1-69, wherein the sample obtained from the patient is a tissue sample, a whole blood sample, a plasma sample, or a serum sample.

71. The method of any one of claims 1-70, wherein the expression level is an mRNA expression level.

72. The method of claim 71, wherein the mRNA expression level is determined by RNA-Seq, qPCR, microarray analysis, gene expression profiling, serial analysis of gene expression, or whole genome sequencing.

73. The method of claim 72, wherein the mRNA expression level is determined by qPCR.

74. The method of any one of claims 1-70, wherein the expression level is a protein expression level.

75. The method of any one of claims 1-74, wherein the IRAK4-mediated disorder or condition is selected from the group consisting of an immune disorder, an inflammatory disorder, a fibrotic disorder, an eosinophilic disorder, an infection, pain, a central nervous system disorder, an acute kidney injury, a chronic kidney disease, endometriosis, non-alcoholic fatty liver disease, a metabolic syndrome, and obesity.

76. The method of claim 75, wherein the immune disorder is lupus, asthma, atopic dermatitis, rheumatoid arthritis, inflammatory bowel disease (IBD), Crohn's disease, or ulcerative colitis.

77. The method of claim 75, wherein the inflammatory disorder is lupus, asthma, atopic dermatitis, rheumatoid arthritis, inflammatory bowel disease (IBD), Crohn's disease, or ulcerative colitis.

78. The method of claim 76 or 77, wherein the lupus is systemic lupus erythematosus (SLE).

79. The method of claim 76 or 77, wherein the lupus is lupus nephritis.

80. The method of any one of claims 1-79, wherein the IRAK4 pathway inhibitor is an IRAK4 inhibitor, an IRAK1 inhibitor, a toll-like receptor (TLR) inhibitor, an interleukin-1 receptor (IL-1R) inhibitor, an interleukin-33 receptor (IL-33R) inhibitor, or a myeloid differentiation primary response gene 88 (MyD88) inhibitor.

81. The method of claim 80, wherein the IRAK4 pathway inhibitor is an IRAK4 inhibitor.

82. The method of claim 80, wherein the IRAK4 pathway inhibitor is a TLR inhibitor.

83. The method of claim 82, wherein the TLR inhibitor is a TLR7 inhibitor, a TLR8 inhibitor, a TLR9 inhibitor, a TLR1 inhibitor, a TLR2 inhibitor, a TLR4 inhibitor, a TLRS inhibitor, a TLR6 inhibitor, or a TLR10 inhibitor.

84. The method of claim 83, wherein the TLR inhibitor is a TLR7 inhibitor, a TLR8 inhibitor, or both a TLR7 and TLR8 inhibitor.

85. The method of claim 83, wherein the TLR inhibitor is a TLR9 inhibitor.

86. The method of any one of claims 80-85, wherein the IRAK4 pathway inhibitor is a small molecule inhibitor.

87. The method of any one of claims 18-34 and 52-86, further comprising administering to the patient an additional therapeutic agent.

88. The method of claim 87, wherein the additional therapeutic agent is a corticosteroid, a nonsteroidal anti-inflammatory drug (NSAID), chloroquine, hydroxychloroquine (PLAQUENIL®), cyclosporine, azathioprine, methotrexate, mycophenolate mofetil (CELLCEPT®), or cyclophosphamide (CYTOXAN®).

89. The method of claim 87 or 88, wherein the IRAK4 pathway inhibitor and the additional therapeutic agent are co-administered.

90. The method of claim 87 or 88, wherein the IRAK4 pathway inhibitor and the additional therapeutic agent are sequentially administered.

91. A kit for identifying a patient having an IRAK4-mediated disorder or condition who may benefit from treatment comprising an IRAK4 pathway inhibitor, the kit comprising:
(a) polypeptides or polynucleotides capable of determining the expression level of one or more genes set forth in Table 1; and
(b) instructions for using the polypeptides or polynucleotides to identify a patient having an IRAK4-mediated disorder or condition who may benefit from treatment comprising the IRAK4 pathway inhibitor.

92. The kit of claim 91, wherein the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, PFKFB3, and BCL2A1.

93. The kit of claim 92, wherein the one or more genes set forth in Table 1 comprises one or more genes selected from the group consisting of CD38, SOCS3, AQP9, CDKN1A, GADD45B, B4GALT5, IL15RA, TNFAIP3, SOCS1, IL1RN, and PFKFB3.

94. The kit of claim 93, wherein the one or more genes set forth in Table 1 comprises one or more genes selected from the group consisting of CD38, SOCS3, AQP9, CDKN1A, GADD45B, B4GALT5, IL15RA, TNFAIP3, and SOCS1.

95. The kit of claim 94, wherein the one or more genes set forth in Table 1 comprises all nine of CD38, SOCS3, AQP9, CDKN1A, GADD45B, B4GALT5, IL15RA, TNFAIP3, and SOCS1.

96. The kit of claim 95, wherein the one or more genes set forth in Table 1 comprises all 11 of CD38, SOCS3, AQP9, CDKN1A, GADD45B, B4GALT5, IL15RA, TNFAIP3, SOCS1, IL1RN, and PFKFB3.

97. The kit of claim 96, wherein the one or more genes set forth in Table 1 comprises all 12 of CD38, SOCS3, AQP9, CDKN1A, GADD45B, B4GALT5, IL15RA, TNFAIP3, SOCS1, IL1RN, PFKFB3, and BCL2A1.

98. The kit of claim 97, wherein the one or more genes set forth in Table 1 are all 24 genes set forth in Table 1.


SEQUENCE LISTING (48 sequences)

SEQ ID NO: 1 - RNA, 5694 nt, Homo sapiens
gaggcaagggguugggggugggaagggaaacagagaaaaggcaagugaaacagaaggggaggugcaguuucagaacccagccagccucucucuugcugccuagccuccugccggccucaucuucgcccagccaaccccgccuggagcccuauggccaacugcgaguucagcccgguguccggggacaaacccugcugccggcucucuaggagagcccaacucugucuuggcgucaguauccugguccugauccucgucguggugcucgcgguggucgucccgagguggcgccagcaguggagcgguccgggcaccaccaagcgcuuucccgagaccguccuggcgcgaugcgucaaguacacugaaauucauccugagaugagacauguagacugccaaaguguaugggaugcuuucaagggugcauuuauuucaaaacauccuugcaacauuacugaagaagacuaucagccacuaaugaaguugggaacucagaccguaccuugcaacaagauucuucuuuggagcagaauaaaagaucuggcccaucaguucacacagguccagcgggacauguucacccuggaggacacgcugcuaggcuaccuugcugaugaccucacaugguguggugaauucaacacuuccaaaauaaacuaucaaucuugcccagacuggagaaaggacugcagcaacaacccuguuucaguauucuggaaaacgguuucccgcagguuugcagaagcugccugugaugugguccaugugaugcucaauggaucccgcaguaaaaucuuugacaaaaacagcacuuuugggaguguggaaguccauaauuugcaaccagagaagguucagacacuagaggccugggugauacaugguggaagagaagauuccagagacuuaugccaggaucccaccauaaaagagcuggaaucgauuauaagcaaaaggaauauucaauuuuccugcaagaauaucuacagaccugacaaguuucuucagugugugaaaaauccugaggauucaucuugcacaucugagaucugagccagucgcugugguuguuuuagcuccuugacuccuugugguuuaugucaucauacaugacucagcauaccugcuggugcagagcugaagauuuuggaggguccuccacaauaaggucaaugccagagacggaagccuuuuuccccaaagucuuaaaauaacuuauaucaucagcauaccuuuauugugaucuaucaauagucaagaaaaauuauuguauaagauuagaaugaaaauuguauguuaaguuacuucacuuuaauucucaugugauccuuuuauguuauuuauauauugguaacauccuuucuauugaaaaaucaccacaccaaaccucucuuauuagaacaggcaagugaagaaaagugaaugcucaaguuuuucagaaagcauuacauuuccaaaugaaugaccuuguugcaugauguauuuuuguacccuuccuacagauagucaaaccauaaacuucauggucaugggucauguuggugaaaauuauucuguaggauauaagcuacccacguacuuggugcuuuaccccaacccuuccaacagugcugugagguugguauuauuucauuuuuuagaugagaaaaugggagcucagagagguuauauauuuaaguuggugcaaaaguaauugcaaguuuugccaccgaaaggaauggcaaaaccacaauuauuuuugaaccaaccuaauaauuuaccguaaguccuacauuuaguaucaagcuagagacugaauuugaacucaacucuguccaacuccaaaauucaugugcuuuuuccuucuaggccuuucauaccaaacuaauaguaguuuauauucucuuccaacaaaugcauauuggauuaaauugacuagaauggaaucuggaauauaguucuucuggauggcuccaaaacacauguuuuucuucccccgucuuccuccuccucuucaugcucaguguuuuauauauguaguauacaguuaaaauauacuuguugcugguacuggcagcuuauauuuucucucuuuuuucauggauuaaccuugcuugagggcuuuaacaauuguauuacuuuuucaaagaacuaagcuuuagcuucauugauuuuuuucuauuuaauuggguuuugcucuucucuuuagcauuggaaacauagaaaugcuuucugauuucuuuggguagauuuacguauucagcuucuugagauggaaguuuagaucacugauccuucagcuuguuuucuuuuuuguauacauagauuuuaggacgauauauuuucccuugaguucugcuuuagcugcagcucuuauguuuugauaugccucucuuuauuauccuucaguuaaaaauaucuuucaauucauuguuauauaaaaauaugugccuaguuuuuaacaucuggagauuuucuaguuuugaaaaaaacauaagccaggcaugguggcucacaccuguauccccagcacuuugggaggccgagacgggaggaucgccugagcucaggaguuuuuacaccagccugggaauaacagugagacauuaucuccaaaaaaauuaccuggguaugguguugugcaccuguagucccagcuacucuggagacugaggugggaggauuguuugagcuugggagguugaggcugcagggagcugugaucacaccacugcacucuggccugagugacagauugagacccugucucaauaaaagcaaaaauaaagaaaauaaaccauauguguugaacaaaggauuaauaaauuaauuugagacuccuucagggaaugaccacaauuuauugaaaauagccuaaauguuggagucaggcauuucuggauucauauuuugacaucaugcugucaucuugaacaaaaugccuaaccuuucugaacuucaacuuccuugccacucaaauaaggauuacaaaacuuaaaaugugguaaguacuaaagacgacagcaaaaauugaguccagcacagagcuuccuaaauaagcaagcacucaacagaguugguuccuuucuuccuccccugcuugacaauccaguuucccacaggagccuuuguagcuguagccaccauggucaguccagggauucuucacuagccccuucuccccuggcagacauccuugugggaguuuagucuuggcucgacaugaggauggggguuugggaccaguucugagugagaaucagacuugccccaaguugccauuagcucccccugcagaaugucuucagaaucggggcccggucagucuccugggugaccugcuguuuuccucuuaagauccuuuccacuuugguugcugcuuucgggacucaucgaguccuugcucaacaggauaccccuugaaguggcugccugggccacauccccuuccaaacaagaaaucaaaauauuagaaaucaauuuuugaaauuuccccuaggaagacucauuugaguguucaaguucagagccaguggagaccuuaggggaggguggucacaaggauuuugcacagugcuuuagagggucccagggagccacagagguggugaggggcugggugcucuuuucuccgugcaugaccuugugugucuaucuucauuaccacaaugccucaucucuaccuccuuucccccuguaguuccaacguggguaucuuugccaucucuggcccgaaggacuuucugaccuacauguauaaauacccccucacaauauauauuacuuuuccuauaagugacuucucuacuggauuacugguugcucauacaccucauauuuuacucguaaaucuacuacucccugucugccuacuccauucucauuugcuguagaaaauucucuuaccaucccaacuuucacccaccaucaugcuuacccaaaggcugugggaaugaccugggcccuaaugccccuuuucuaaauuccuaaggcucaccauuuuccuauuguaaugguucuugaccuuauaauguuugaggcaccuuuucaaauauaguccuuugauuucagacugaauacuugaaaggacacacacacacauacguaagugcauaugacugcauacacccacacacacacacgugccuguauacagucauaugauacauacacaaacacacgcacacaagccugcauacaucauaugccaacaguggggauauguucugagaaaugcaucauuagaugauuuugucauugugugaacaucauagaguguacuuacacuaaccuagauggucuaaccuacuacacacccaggcuacaugguaucaccuauuccuccuaggcuacaagccuguacagcgugugucuguacuaaaugcugugggcaauuuuaaccugaugguaaauguuuguguaucuaaacauaucuaaacauagaaaagguacaguaaacaugcaguauuauaaucuuaugagaccgucaucauauaugugguccacuguuugggccaucauuggcugaaaagugguuaugcgacacaugacuguauauauacuuuccuguuacaacaacagugucucucaauccacaguaauugcagcauccaguaggucuuacuuuagcccugagucaccauuugugucaacguguuuagugccauguccacgucucucauguaacuggcagagcuaucaaauauuuuggcaaaacacauuguuucuuuggcuuugccuugguaacuuucugugccuuuuguagcucuuguuuggaagaagcucaacccaugucugcacacugugauacaagggggacagcaucgacaucgacuuacuucuuggugccuuauuccuccuuagaacaauuccuaaaucuguaacuuaaguuucucaggaagauuccauacugcacagaaaacugcuuuuguggguuuuuaaaaggcaaguuguuauaugugcuggauaguuuuuaaguaugacauaaaaauuguauaaaguaaaauauuaaaauacaccuagaauacuguauaacuuuaagucauuuuaucaacacauugcuaauccagauauuuucccgcaguuuuucuuugaauaacagagcaauuaauuuacuuuuacuaugaagagucaucauuuuaguauguauuuuaagcaauccaccaagaacucaguaggcagcugagaggugcugcccagagaaguggugauuagcuuggccuuagcucacccacacaaagcacaacaggcuuugaacuauucccuaacggggcauuuauucuuuuuuuuuuuuuuuuuugggagacggagucucgcugucgcccaggcuagagugcaguggcgcgaucucggcucacugcaggcuccacccccugggguucacgccauucuccugccucagccucccaaguagcugggacugcaggcgcccgccaucucgcccggcuaauuuuuuguauuuuuaguagagacgggguuucaccguguuagccaggauagggcauuuauucuugaacuugauucagagaggcacacauuaccauucucuaaucagaaugcaaguagcgcaaggcgguggaaacuauggaauucggaggcaggugaugcauugggcgaguuuauuaacaucugugacucucuaguuugaaauuuauuuguaacagacaaaaaugaauuaaacaaacaauaaaaguauaauaaagaacugugcugaau

SEQ ID NO: 2 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 3 - RNA, 2737 nt, Homo sapiens
gcggcuccgacuuggacucccugcuccgcugcugccgcuucggccccgcacgcagccagccgccagccgcccgcccggcccagcucccgccgcggccccuugccgcggucccucuccugguccccucccgguugguccgggggugcgcagggggcagggcgggcgcccaggggaagcucgagggacgcgcgcgcgaaggcuccuuuguggacuucacggccgccaacaucugggcgcagcgcgggccaccgcuggccgucucgccgccgcgucgccuuggggacccgagggggcucagccccaaggacggagacuucgauucgggaccagccccccgggaugcgguagcggccgcugugcggaggccgcgaagcagcugcagccgccgccgcgcagauccacgcuggcuccgugcgccauggucacccacagcaaguuucccgccgccgggaugagccgcccccuggacaccagccugcgccucaagaccuucagcuccaagagcgaguaccagcugguggugaacgcagugcgcaagcugcaggagagcggcuucuacuggagcgcagugaccggcggcgaggcgaaccugcugcucagugccgagcccgccggcaccuuucugauccgcgacagcucggaccagcgccacuucuucacgcucagcgucaagacccagucugggaccaagaaccugcgcauccagugugaggggggcagcuucucucugcagagcgauccccggagcacgcagcccgugccccgcuucgacugcgugcucaagcuggugcaccacuacaugccgcccccuggagcccccuccuuccccucgccaccuacugaacccuccuccgaggugcccgagcagccgucugcccagccacucccugggaguccccccagaagagccuauuacaucuacuccgggggcgagaagaucccccugguguugagccggccccucuccuccaacguggccacucuucagcaucucugucggaagaccgucaacggccaccuggacuccuaugagaaagucacccagcugccggggcccauucgggaguuccuggaccaguacgaugccccgcuuuaagggguaaagggcgcaaagggcaugggucgggagaggggacgcaggccccucuccuccguggcacauggcacaagcacaagaagccaaccaggagagaguccuguagcucuggggggaaagagggcggacaggccccucccucugcccucucccugcagaauguggcaggcggaccuggaauguguuggagggaagggggaguaccaccugagucuccagcuucuccggaggagccagcuguccuggugggacgauagcaaccacaaguggauucuccuucaauuccucagcuuccccucugccuccaaacaggggacacuucgggaaugcugaacuaaugagaacugccagggaaucuucaaacuuuccaacggaacuuguuugcucuuugauuugguuuaaaccugagcugguuguggagccugggaaagguggaagagagagagguccugagggccccagggcugcgggcuggcgaaggaaauggucacaccccccgcccaccccaggcgaggauccuggugacaugcuccucucccuggcuccggggagaagggcuuggggugaccugaagggaaccauccugguaccccacauccucuccuccgggacagucaccgaaaacacagguuccaaagucuaccuggugccugagagcccagggcccuuccuccguuuuaagggggaagcaacauuuggaggggauggaugggcuggucagcuggucuccuuuuccuacucauacuauaccuuccuguaccuggguggauggagcgggaggauggaggagacgggacaucuuucaccucaggcuccugguagagaagacaggggauucuacucugugccuccugacuaugucuggcuaagagauucgccuuaaaugcucccugucccauggagagggacccagcauaggaaagccacauacucagccuggauggguggagaggcugagggacucacuggagggcaccaagccagcccacagccagggaaguggggagggggggcggaaacccaugccucccagcugagcacugggaaugucagcccaguaaguauuggccagucaggcgccucguggucagagcagagccaccaggucccacugccccgagcccugcacagcccucccuccugccugggugggggaggcuggaggucauuggagaggcuggacugcugccaccccgggugcucccgcucugccauagcacugaucagugacaauuuacaggaauguagcagcgauggaauuaccuggaacaguuuuuuguuuuuguuuuuguuuuuguuuuugugggggggggcaacuaaacaaacacaaaguauucugugucagguauugggcuggacagggcaguuguguguuggggugguuuuuuucucuauuuuuuuguuuguuucuuguuuuuuaauaauguuuacaaucugccucaaucacucugucuuuuauaaagauuccaccuccaguccucucuccuccccccuacucaggcccuugaggcuauuaggagaugcuugaagaacucaacaaaaucccaauccaagucaaacuuugcacauauuuauauuuauauucagaaaagaaacauuucaguaauuuauaauaaagagcacuauuuuuuaaugaaaaac

SEQ ID NO: 4 - protein, 225 aa, Homo sapiens
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL

SEQ ID NO: 5 - RNA, 3031 nt, Homo sapiens
acugugauuuggcaacacaacuggcacaucucuuuucucaucucuugaaaaaaaccaacagagaaaaaaguaccuugagaauaaagguaaugauuaaucugucaggcacaaaagggauuguuuuggggauuucggguucuaagucgcagauucaaacaaauagcagcgaacagggaaugacaguuccaccagaagacgauuaagccacagccucuaauuggaacggcauuuguacagucagagacucuuaccagacaucuccaggaaucugugagccauugucaaaacguccauuuucaucuggcugugaaagugaggaccacaacagguagguauugguagaaacaggaguccucagagaagccccaagaugcagccugagggagcagaaaagggaaaaagcuucaagcagagacuggucuugaagagcagcuuagcgaaagaaacccucucugaguucuugggcacguucaucuugauuguccuuggauguggcuguguugcccaagcuauucucagucgaggacguuuuggaggggucaucacuaucaauguuggauuuucaauggcaguugcaauggccauuuauguggcuggcggugucucugguggucacaucaacccagcugugucuuuagcaaugugucucuuuggacggaugaaaugguucaaauugccauuuuaugugggagcccaguucuugggagccuuugugggggcugcaaccgucuuuggcauuuacuaugauggacuuauguccuuugcugguggaaaacugcugaucgugggagaaaaugcaacagcacacauuuuugcaacauacccagcuccguaucuaucucuggcgaacgcauuugcagaucaagugguggccaccaugauacuccucauaaucgucuuugccaucuuugacuccagaaacuugggagcccccagaggccuagagcccauugccaucggccuccugauuauugucauugcuuccucccugggacugaacaguggcugugccaugaacccagcucgagaccugagucccagacuuuucacugccuuggcaggcuggggguuugaagucuucagagcuggaaacaacuucugguggauuccuguagugggcccuuugguuggugcugucauuggaggccucaucuauguucuugucauugaaauccaccauccagagccugacucagucuuuaagacagaacaaucugaggacaaaccagagaaauaugaacucagugucaucauguaguggcaugcucagcucuggauuugcagucaguuugggauucucuucagaaagauggcaucuaagugucuguguucuuguaagccugagguggaauccacccaguuuugucugcuagccauaugggacaucuaauuggaaaagcaucugcauaaaaguuuggaaacaaugaccacuucucuaccauugucccccacccccaccccccagaauaacgcugacuguccccugaaacagccuucucuccugcccuguuuauuucauccucgaugggaauucuugcuagguaagcacuaauaacucggcaucuugacgauagucccauuugggugguuucagcugcacuaucuguaugaaauggugucaccaaaacccuuuucuucaguaucgacaaagauuacauucugaguaccaaccaaacccuaaauugaaagacaaaacuaugguuucagucaacauauucaugaauuagggagcuaauggguuaagcuuccaguucccgcuaugcuacuggauuuguauaaauacugauauucuccaaaccuagugguguagggagcaagagaaugcagcuggaaggcacaaggggaggacauuguggcauucagaaacugcaggagacaagaugaauuugagaagccaaauggaauuuuuaauggaaaccauuuaucagauuaaucucuugcucuccugcauuuuagaggacaccaauuaauuuccuggucuuuaguauauaauaaccuaaaauaccauuguaaccucagucaugaaaaauacaucacucugucuuuuuagcucaaauguauuuuccuaauugcccacuugagaacagacauuugacaaguuauaucaacgacugugcuuguccauuauuuuacacaugcccuagaagccaaaacugaaagccacuggauccuggucuagcugaaucuucagagugggaggucuccaaaaagauauuaccuuauugggcuuaacaauucacaaggcacuuucacacccauuaucuaauuuaauccucauaaugacuaugugaggcaaaugccacauugcccauuuuucagauaaagaaacaaaaucuuagggaagauaaguugaguuguccaagagcacacugaaaguugaauguuaucuaaugcauuccucuaccuuucagaagaucaguagcuggcugacaaucuuugccaaaucuuccuugcuagccagaaguggaauuggcagcuucuagaauauguacaccucuggacaaaauguuccucaaucuuaagauacaaagacccucauugucugggucuauucccacacuuacugaguacagaugaaggaaagugguagcaauuuaaucauaacuuucauuugcugaaaaacauuaugagaaggccucccuuccuaagccaccucuggucuugcuaagucuugaucuugcuuccugccagcaccaaacauuacauucaggggauuuccucuggcucagucuuuuccccuugaaguucucuaauagauguuacuuuugacaaaagaucgccuaugaguuacaagcaccaggggaugcucuacaucaagggaugcaccuucagucaaacugucaaaaagcccagaauucccaaaggcauuagguuucccaacugcuuugugcugauaucagaacagcagaaauuaaaugugaaauguuucugaugacuuauguucuacaaucuauggacauacgggauuuuuuuuucuugcuuugaagcuaccuggauauuuccuauuugaaauaaaauuguucggucauug

SEQ ID NO: 6 - protein, 295 aa, Homo sapiens
MQPEGAEKGKSFKQRLVLKSSLAKETLSEFLGTFILIVLGCGCVAQAILSRGRFGGVITINVGFSMAVAMAIYVAGGVSGGHINPAVSLAMCLFGRMKWFKLPFYVGAQFLGAFVGAATVFGIYYDGLMSFAGGKLLIVGENATAHIFATYPAPYLSLANAFADQVVATMILLIIVFAIFDSRNLGAPRGLEPIAIGLLIIVIASSLGLNSGCAMNPARDLSPRLFTALAGWGFEVFRAGNNFWWIPVVGPLVGAVIGGLIYVLVIEIHHPEPDSVFKTEQSEDKPEKYELSVIM

SEQ ID NO: 7 - RNA, 2175 nt, Homo sapiens
guuguauaucagggccgcgcugagcugcgccagcugaggugugagcagcugccgaagucaguuccuuguggagccggagcugggcgcggauucgccgaggcaccgaggcacucagaggaggcgccaugucagaaccggcuggggauguccgucagaacccaugcggcagcaaggccugccgccgccucuucggcccaguggacagcgagcagcugagccgcgacugugaugcgcuaauggcgggcugcauccaggaggcccgugagcgauggaacuucgacuuugucaccgagacaccacuggagggugacuucgccugggagcgugugcggggccuuggccugcccaagcucuaccuucccacggggccccggcgaggccgggaugaguugggaggaggcaggcggccuggcaccucaccugcucugcugcaggggacagcagaggaagaccauguggaccugucacugucuuguacccuugugccucgcucaggggagcaggcugaaggguccccagguggaccuggagacucucagggucgaaaacggcggcagaccagcaugacagauuucuaccacuccaaacgccggcugaucuucuccaagaggaagcccuaauccgcccacaggaagccugcaguccuggaagcgcgagggccucaaaggcccgcucuacaucuucugccuuagucucaguuugugugucuuaauuauuauuuguguuuuaauuuaaacaccuccucauguacauacccuggccgcccccugccccccagccucuggcauuagaauuauuuaaacaaaaacuaggcgguugaaugagagguuccuaagagugcugggcauuuuuauuuuaugaaauacuauuuaaagccuccucaucccguguucuccuuuuccucucucccggagguugggugggccggcuucaugccagcuacuuccuccuccccacuuguccgcugggugguacccucuggagggguguggcuccuucccaucgcugucacaggcgguuaugaaauucacccccuuuccuggacacucagaccugaauucuuuuucauuugagaaguaaacagauggcacuuugaaggggccucaccgagugggggcaucaucaaaaacuuuggaguccccucaccuccucuaagguugggcagggugacccugaagugagcacagccuagggcugagcuggggaccugguacccuccuggcucuugauaccccccucugucuugugaaggcagggggaaggugggguccuggagcagaccaccccgccugcccucauggccccucugaccugcacuggggagcccgucucaguguugagccuuuucccucuuuggcuccccuguaccuuuugaggagccccagcuacccuucuucuccagcugggcucugcaauuccccucugcugcugucccucccccuuguccuuucccuucaguacccucucagcuccagguggcucugaggugccugucccacccccacccccagcucaauggacuggaaggggaagggacacacaagaagaagggcacccuaguucuaccucaggcagcucaagcagcgaccgcccccuccucuagcugugggggugagggucccaugugguggcacaggcccccuugagugggguuaucucuguguuagggguauaugaugggggaguagaucuuucuaggagggagacacuggccccucaaaucguccagcgaccuuccucauccaccccaucccuccccaguucauugcacuuugauuagcagcggaacaaggagucagacauuuuaagaugguggcaguagaggcuauggacagggcaugccacgugggcucauauggggcugggaguaguugucuuuccuggcacuaacguugagccccuggaggcacugaagugcuuaguguacuuggaguauuggggucugaccccaaacaccuuccagcuccuguaacauacuggccuggacuguuuucucucggcuccccauguguccugguucccguuucuccaccuagacuguaaaccucucgagggcagggaccacacccuguacuguucugugucuuucacagcuccucccacaaugcugaauauacagcaggugcucaauaaaugauucuuagugacuuuacuuguaaaaaaaaaaaaaaaaaa

SEQ ID NO: 8 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 9 - RNA, 1393 nt, Homo sapiens
ucagaucgccgaagcgucggacuaccguugguuuccgcaacuuccuggauuauccucgccaaggacuuugcaauauauuuuuccgccuuuucuggaaggauuucgcugcuucccgaaggucuuggacgagcgcucuagcucugugggaagguuuugggcucucuggcucggauuuugcaauuucucccuggggacugccguggagccgcauccacuguggauuauaauugcaacaugacgcuggaagagcucguggcgugcgacaacgcggcgcagaagaugcagacggugaccgccgcgguggaggagcuuuugguggccgcucagcgccaggaucgccucacaguggggguguacgagucggccaaguugaugaauguggacccagacagcgugguccucugccucuuggccauugacgaggaggaggaggaugacaucgcccugcaaauccacuucacgcucauccaguccuucugcugugacaacgacaucaacaucgugcgggugucgggcaugcagcgccuggcgcagcuccugggagagccggccgagacccagggcaccaccgaggcccgagaccugcauugucuccuggucacgaacccucacacggacgccuggaagagccacggcuugguggagguggccagcuacugcgaagaaagccggggcaacaaccaguggguccccuacaucucucuucaggaacgcugaggcccuucccagcagcagaaucuguugaguugcugccacaaacaaaaaauacaauaaauauuugaacccccucccccccagcacaacccccccaaaacaacccaacccacgaggaccaucgggggcagagucguuggagacugaagaggaagaggaggaggagaaggggagugagcggccgcccccagggcggagauccaggagcuggcggccgccgauccgauggagaaggggggacccaggccagcaggagacaggacccccgaagcugaggccuugggauggagcagaagccggaguggcggggcacgcugccgccuuccccaucacggaggguccagacuguccacucggggguggagugagacugacugcaagccccacccuccuugagacuggagcuggcgucugcauacgagagacuugguugaacuugguugguccuugucugcacccucgacaagaccacacuuugggacuugggagcuggggcugaaguugcucuguacccaugaacucccaguuugcgaauuauagagacaaucuauuuuguuacuugcacuuguuauucgaaccacugagagcgagaugggaagcauagauaucuauauuuuuauuucuacuaugagggccuuguaauaaauuucuaaagccucugaaaaaaaaaaaaaaaaaa

SEQ ID NO: 10 - protein, 160 aa, Homo sapiens
MTLEELVACDNAAQKMQTVTAAVEELLVAAQRQDRLTVGVYESAKLMNVDPDSVVLCLLAIDEEEEDDIALQIHFTLIQSFCCDNDINIVRVSGMQRLAQLLGEPAETQGTTEARDLHCLLVTNPHTDAWKSHGLVEVASYCEESRGNNQWVPYISLQER

SEQ ID NO: 11 - RNA, 4743 nt, Homo sapiens
cgcuucauguggcccggcccgcgacggccggcggcugggagcggcgaggcggcggcggcggcgaguggcggcccgcgaggcccgggaggcgguggccgaggcccaggcgguggcggcggcggcccaggaggcggcggacggggagcugcgggagcaggcccggccuggcucucuagcggccgccuggcugcagcaugcgcgcccgccgggggcugcugcggcugccgcgccgcucgcugcucgccgcgcucuucuucuuuucucucucguccucgcugcuguacuucgucuauguggcgcccggcauagugaacaccuaccucuucaugaugcaagcccaaggcauucugauccgggacaacgugagaacaaucggugcucagguuuaugagcaggugcuucggagugcuuaugccaagaggaacagcaguguaaaugacucagauuauccucuugacuugaaccacagugaaaccuuccugcaaacuacaacauuucuuccugaagacuucaccuacuuugcaaaccauaccugcccugaaagacucccuuccaugaagggcccaauagacauaaacaugagugaaauuggaauggauuacauucaugaacucuucuccaaagacccaaccaucaagcucggaggucacuggaagccuucugauugcaugccucgguggaagguggcgauccuuauccccuuccggaaccgccacgagcaccucccaguccuguucagacaccugcuucccaugcuccagcgccagcgcuugcaguuugcauuuuaugugguugaacaaguugguacccaacccuuuaaucgagccaugcuuuucaacguuggcuuucaagaggcaaugaaagacuuggauugggacuguuugauuuuucaugauguagaucacauaccggaaagugaucgcaacuauuauggauguggacagaugccgaggcauuuugcaaccaaauuggauaaguauauguaucugcuuccuuauaccgaguucuuuggcggagugaguggcuuaacaguggaacaauuucggaaaaucaauggcuuuccuaaugcuuucugggguugggguggagaagaugacgaccucuggaacagaguacagaaugcaggcuauucugugagccggccagagggugacacaggaaaguacaaguccauuccucaucaccaucgaggagaaguccaguuucuuggaagguaugcucugcugaggaagucaaaagaacggcaagggcuggauggccucaacaaccugaacuacuuugcaaacaucacauacgacgccuuguauaaaaacauaacugucaaccugacacccgagcuggcucaggugaacgaguacugagaggagagaauguacguuugcuuuacccaccgccaccaagaaagcaguccgaugagauuuuuuuuuggaggggggagggucuacacagcaagagaacagaaauacugugucucaugaaggaucacagaguucagggggaaaaugugacagcacacgcacaaacgccuucacuggaucagccgcuggaacugagggagugagcuuggggacuuccuucgucagcacuggcuuucuguuuucacaagacagacgucugucccgcugcucucuccccaucuccuaccccacauccugucuuagccgcagucuccagaacccaugaugaacugugaucugccgugguccugccgugguccugccguggagccugucccuacacaugaccuuggagccucuuggccuucagagcagaggcaaacccaccacagggcagcugcguuuuaggaagagcaaaugaaacuccacaccauucuucuagaucucugguguucucuuugguuucauuuuuuuaaaaaauuaccuucuuuggguggggauugaggguggaggggaggguguuugggaaagauaaauagacauaaauauauaacaaucacuucuugaagaaguauaauuguaaauaagccauguaaaaugccuuuuuaaaauuuaauuuucuagcuggcuccaauucaaauugaggauuuauguauuaggccacuuacuugguuggcaagugcaggaacucaguuaaaaugcaguugaagaaugucaucucccgaauugcugucacuuuggcgagggaguggauauagggcaugucacaaaagaacaaaauaacccgaccuuuauugcugggagcuggcuucugucccuuucuucccccccccacgagucuugcccuugacuucugcucuggauucacucuucccugucggccgcgcaugugcucaucccacucuccgcuaagcgggaggcugcuguuagagcaggcugcuuccugccuaaagcaggcccuucggggcucgcugcacacacaucucuggcucuccaggcuucguguucugucuuuucaucagcauggcggggcggggggcggggggcggggguguguaugggaaucccucccccucuuacuuuuucucuuguggaacuuggccacaguuucugaacaaugugccuacauuaccagcuggcuucagugauuccucugugucccuuuuugguuucuggaaagauucuuugucaacauuaguaacugauacauagaaccaaggagcacucaaauagggagccaggagccagggagcuggugacacuugugugcuguggggcagcugggauccagguaagaccggauugaagcuuugaaauuagacuaacaaagcuccagacagcaagagcccaggugcacugcucacacccccaccugcauuuugaagucauauuauuuuuuguuuuguuuuuuaagacggucuggcucugucgccuaagcuggagugugguggcacgaucacagcucacugcagccuccaucuccuaggcucaagccauuuucccaccucagccucccgaguagcugggacuacaggugcacaccaccacaccuggcuaauuuuuuguauuuuuaguagagacagggguuucuuccauguugcccaggcuggucucgaacuccuggacucaagcaauccgcccaccuugacuucccaaagugcugggauuaugggcgggugugagccauugcgcccagccuugaagucauguucuaaauuguauuugaauuugugccucuuuguuuuuccccaaaccaaagcccucaaauuguagucucugucggcuucugcagaauucuggaaaaugccaguuuuccucccccgcccuuguuuuccauaaaacauauuuauauauugugaugaggaguacuuucugaagaguacuucguauuuuuuuuuaauugccuuguuugccuucaacuuccuugauuuucauaguuuacauggguguguguagggguguguguguguauguguguggguuagggcuuuuuucguugcaugugaugguucuguggacauaugauccccacaaacugugggagugauuggccaggccuuguuuuguuuguuuguuuguuuguguuuuuguucuuuugaagaauagagugguauuuagaaaauaaauugcauugcaaagcucuuaucggcucauaugagagagcagguuccugcccuugaaaaugccgguaagcuauagcauauguuuuuuaagacuuaagcauuucaugcuuuaaaauaccuucacaagugaacauuacacacagaaguucauuugguuuuccuuuguuuuauggugcauauagcaauaaagaccccccuccacccugcaacccccaucccccaccgggccuuugucccugccuuggcuuuucuccccuucucauucuccucuccccuuuccucacugaaggcugugaguugcuuucaaugugacaacacuaugaugucauuuggaaggauuugccaggacagacugauucugaguccugggugccguauguguaugcggcaguguugucaggcgaucuuguuugaagcucuauguugccauaauuaccaucaaguacacacuguuggcaaaaggcuaacaccugacuuuagaaaaugcugauuugagaacaaaaggaaaggucuuuuuucacugcuuaaaguggggucacuuugauaccuuugcggucaugucugugucugaugaguguagaaucucuggaugugcacugucagucauguguccaccaggccucgaauaucauaugggaaaugucauaguuaaaaacguacagccaggcccgugugcuguuaauagugugaaauugucauguuaaaaaaaaaaacaggaaccaaaugugaccuugugcauauauugguagcugaaaaucuucaaggcuacugauggguggccccuuaaucuugucuuugauugcugugugcagggaaagguguccccguuuguucaugcuguuuugggggguggggggguauuugcaagaauacucauuuugacauaauagguccucuugucagagauccucuaccacagacauuaauagcugagcaggagccacauggauugauuguauccacucaccauugacgauggcauugagcguagcuagcuuauuuccaucacuacguguuuuugagcuugcucuuacguuuuaagaggugccagggguacauuuuugcacugaaaucuaaagauguuuuaaaaaacacuuuucacaaaaauaguccuuugucauuacauuauuuacucauguguuuguacauuuuuguauguuaauuuaugaaugauuuuuucaguaaaaaauacauauucaagaaccaaaaaaaaaaaaaaaaaa

SEQ ID NO: 12 - protein, 388 aa, Homo sapiens
MRARRGLLRLPRRSLLAALFFFSLSSSLLYFVYVAPGIVNTYLFMMQAQGILIRDNVRTIGAQVYEQVLRSAYAKRNSSVNDSDYPLDLNHSETFLQTTTFLPEDFTYFANHTCPERLPSMKGPIDINMSEIGMDYIHELFSKDPTIKLGGHWKPSDCMPRWKVAILIPFRNRHEHLPVLFRHLLPMLQRQRLQFAFYVVEQVGTQPFNRAMLFNVGFQEAMKDLDWDCLIFHDVDHIPESDRNYYGCGQMPRHFATKLDKYMYLLPYTEFFGGVSGLTVEQFRKINGFPNAFWGWGGEDDDLWNRVQNAGYSVSRPEGDTGKYKSIPHHHRGEVQFLGRYALLRKSKERQGLDGLNNLNYFANITYDALYKNITVNLTPELAQVNEY

SEQ ID NO: 13 - RNA, 1664 nt, Homo sapiens
ucucugcaauccgggggcuaaagcaaaagcgaaagcgaauacuccuuagcagcggucugguccuggagcugagcucgccaccucccagggaccccagccuugcgucccguugggucacugcuggggacaauuggccauggccucgccgcagcuccggggcuauggaguccaggccauuccuguguugcugcugcugcuguugcuacuguugcucccgcugagggugacgccgggcaccacguguccaccucccguaucuauugagcaugcugacauccgggucaagaauuacagugugaacuccagggagagguaugucuguaacucuggcuuuaagcggaaagcuggaacauccacccugauugagugugugaucaacaagaacacaaauguugcccacuggacaacucccagccucaagugcaucagagaccccucccuagcucacuacaguccagugccaacaguagugacaccaaaggugaccucacagccagagagccccucccccucugcaaaagagccagaagcuuucucucccaaaucagauaccgcaaugaccacagagacagcuauuaugccuggcuccaggcugacaccaucccaaacaacuucugcaggaacuacagggacaggcagucacaaguccucccgagccccaucucuugcagcaacaaugaccuuggagccuacagccuccaccucccucaggauaacagagauuucuccccacaguuccaaaaugacgaaaguggccaucucuacaucgguccucuugguuggugcagggguugugauggcuuuccuggccugguacaucaaaucaaggcagccuucucagccgugccguguugagguggaaaccauggaaacaguaccaaugacugugagggccagcagcaaggaggaugaagacacaggagccuaaccacacgauuucagaaacucagggagaccugcccagcugauccugaagagaagauacaagggcaguuaguccagcugcgccuggacacaggagaaagcccaaugugauaauagaggucucugguaugaucuguuuuacugagccugggugccucccauauucaggccuggauccccggaguccggaaggacaccaccuuucuuccugguucaucacagaggccaacuucccagaguacaagcagccugagcucuccugggagucuccauuaaaagccucgaguuccacucagagauuaacucaaagcagcugaucugcuagacucuuuuucuauucccuacucugacuugcaguuuacagagauugacaaggcucccauugucuuccaaggcuccucuggcacaggagaugucuguaaagaagacagcaacauuugagcuccugaagacuuggcccuuugcugcuuugcaccuauuggaggagagcagagaacagaagaagagauacugagccaaugaacccuuucguauaggauucaugacaaaaccaaacucagugacuauauauguauguguguguguguguguauguaaaaguguauauuuacauauacauuuauauuuauacuuucuuuucuauuauaucuacauauuguauaugauuuauauuugaaagugcuuuguguagacaaaauaaaauaucuauuuucaguacaaaaagcauuaaaauuaucaac

SEQ ID NO: 14 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 15 - RNA, 4753 nt, Homo sapiens
cuuuggaaagucccguggaaauccccgggccuacaacccgcauacaacugaaacggggcaaagcagacugcgcagucugcagucuucguggcgggccaagcgagcuuggagcccgcgggggcggagcggugagagcggccgccaagagagaucacacccccagccgacccugccagcgagcgagcccgaccccaggcguccauggagcgucgccuccgcccggucccugccccgacccccgccugcggcgcgcuccugccuugaccaggacuugggacuuugcgaaaggaucgcggggcccggagagguaaccgccgcgccucccggagagguaaccgccgcgccucccggagagguguuggagagcacaauggcugaacaaguccuuccucaggcuuuguauuugagcaauaugcggaaagcugugaagauacgggagagaacuccagaagacauuuuuaaaccuacuaaugggaucauucaucauuuuaaaaccaugcaccgauacacacuggaaauguucagaacuugccaguuuuguccucaguuucgggagaucauccacaaagcccucaucgacagaaacauccaggccacccuggaaagccagaagaaacucaacuggugucgagaaguccggaagcuuguggcgcugaaaacgaacggugacggcaauugccucaugcaugccacuucucaguacauguggggcguucaggacacagacuugguacugaggaaggcgcuguucagcacgcucaaggaaacagacacacgcaacuuuaaauuccgcuggcaacuggagucucucaaaucucaggaauuuguugaaacggggcuuugcuaugauacucggaacuggaaugaugaaugggacaaucuuaucaaaauggcuuccacagacacacccauggcccgaaguggacuucaguacaacucacuggaagaaauacacauauuuguccuuugcaacauccucagaaggccaaucauugucauuucagacaaaaugcuaagaaguuuggaaucagguuccaauuucgccccuuugaaaguggguggaauuuacuugccucuccacuggccugcccaggaaugcuacagauaccccauuguucucggcuaugacagccaucauuuuguacccuuggugacccugaaggacagugggccugaaauccgagcuguuccacuuguuaacagagaccggggaagauuugaagacuuaaaaguucacuuuuugacagauccugaaaaugagaugaaggagaagcucuuaaaagaguacuuaauggugauagaaauccccguccaaggcugggaccauggcacaacucaucucaucaaugccgcaaaguuggaugaagcuaacuuaccaaaagaaaucaaucugguagaugauuacuuugaacuuguucagcaugaguacaagaaauggcaggaaaacagcgagcaggggaggagagaggggcacgcccagaaucccauggaaccuuccgugccccagcuuucucucauggauguaaaaugugaaacgcccaacugccccuucuucaugucugugaacacccagccuuuaugccaugagugcucagagaggcggcaaaagaaucaaaacaaacucccaaagcugaacuccaagccgggcccugaggggcucccuggcauggcgcucggggccucucggggagaagccuaugagcccuuggcguggaacccugaggaguccacuggggggccucauucggccccaccgacagcacccagcccuuuucuguucagugagaccacugccaugaagugcaggagccccggcugccccuucacacugaaugugcagcacaacggauuuugugaacguugccacaacgcccggcaacuucacgccagccacgccccagaccacacaaggcacuuggaucccgggaagugccaagccugccuccaggauguuaccaggacauuuaaugggaucugcaguacuugcuucaaaaggacuacagcagaggccuccuccagccucagcaccagccucccuccuuccugucaccagcguuccaagucagaucccucgcggcucguccggagccccuccccgcauucuugccacagagcuggaaacgacgccccugcuggcugccugucucaagcugcacggacuccuggggacaggacggggacgagcaagugcagaaaagccggcugcguguauuuugggacuccagaaaacaagggcuuuugcacacuguguuucaucgaguacagagaaaacaaacauuuugcugcugccucagggaaagucagucccacagcguccagguuccagaacaccauuccgugccuggggagggaaugcggcacccuuggaagcaccauguuugaaggauacugccagaaguguuucauugaagcucagaaucagagauuucaugaggccaaaaggacagaagagcaacugagaucgagccagcgcagagaugugccucgaaccacacaaagcaccucaaggcccaagugcgcccgggccuccugcaagaacauccuggccugccgcagcgaggagcucugcauggagugucagcaucccaaccagaggaugggcccuggggcccaccggggugagccugcccccgaagacccccccaagcagcguugccgggcccccgccugugaucauuuuggcaaugccaagugcaacggcuacugcaacgaaugcuuucaguucaagcagauguauggcuaaccggaaacaggugggucaccuccugcaagaaguggggccucgagcugucagucaucauggugcuauccucugaaccccucagcugccacugcaacagugggcuuaagggugucugagcaggagaggaaagauaagcucuucguggugcccacgaugcucagguuugguaacccgggaguguucccagguggccuuagaaagcaaagcuuguaacuggcaagggaugaugucagauucagcccaagguuccuccucuccuaccaagcaggaggccaggaacuucuuuggacuuggaaggugugcggggacuggccgaggccccugcacccugcgcaucaggacugcuucaucgucuuggcugagaaagggaaaagacacacaagucgcguggguuggagaagccagagccauuccaccuccccucccccagcaucucucagagaugugaagccagauccucauggcagcgaggcccucugcaagaagcucaaggaagcucagggaaaauggacguauucagagaguguuuguaguucaugguuuuucccuaccugcccgguuccuuuccugaggacccggcagaaaugcagaaccauccauggacugugauucugaggcugcugagacugaacauguucacauugacagaaaaacaagcugcucuuuauaauaugcaccuuuuaaaaaauuagaauauuuuacugggaagacguguaacucuuuggguuauuacugucuuuacuucuaaagaaguuagcuugaacugaggaguaaaaguguguacauauauaauauacccuuacauuauguaugagggauuuuuuuaaauuauauugaaaugcugcccuagaaguacaauaggaaggcuaaauaauaauaaccuguuuucugguuguuguuggggcaugagcuuguguauacacugcuugcauaaacucaaccagcugccuuuuuaaagggagcucuaguccuuuuuguguaauucacuuuauuuauuuuauuacaaacuucaagauuauuuaagugaagauauuucuucagcucuggggaaaaugccacaguguucuccugagagaacauccuugcuuugagucaggcugugggcaaguuccugaccacagggaguaaauuggccucuuugauacacuuuugcuugccuccccaggaaagaaggaauugcauccaagguauacauacauauucaucgauguuucgugcuucuccuuaugaaacuccagcuauguaauaaaaaacuauacucuguguucuguuaaugccucugaguguccuaccuccuuggagaugagauagggaaggagcagggaugagacuggcaauggucacagggaaagauguggccuuuugugaugguuuuauuuucuguuaacacuguguccugggggggcugggaaguccccugcaucccaugguacccugguauugggacagcaaaagccaguaaccaugaguaugaggaaaucucuuucuguugcuggcuuacaguuucucugugugcuuugugguugcugucauauuugcucuagaagaaaaaaaaaaaaggaggggaaaugcauuuuccccagagauaaaggcugccauuuugggggucuguacuuauggccugaaaauauuugugauccauaacucuacacagccuuuacucauacuauuaggcacacuuuccccuuagagcccccuaaguuuuucccagacgaaucuuuauaauuucuuuccaaagauaccaaauaaacuucaguguuuucaucuaauucucuuaaaguugauaucuuaauauuuuguguugaucauuauuuccauucuuaaugugaaaaaaaguaauuauuuauacuuauuauaaaaaguauuugaaauuugcacauuuaauugucccuaauagaaagccaccuauucuuuguuggauuucuucaaguuuuucuaaauaaauguaacuuuucacaagagucaacauuaaaaaauaaauuauuuaagaacagaaaaaaaaaaaaaaaa

SEQ ID NO: 16 - protein, 790 aa, Homo sapiens
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

SEQ ID NO: 17 - RNA, 1216 nt, Homo sapiens
ggcagcugcacggcuccuggccccggagcaugcgcgagagccgccccggagcgccccggagccccccgccgucccgcccgcggcgucccgcgccccgccgccagcgcacccccggacgcuauggcccaccccuccggcuggccccuucuguaggaugguagcacacaaccagguggcagccgacaaugcagucuccacagcagcagagccccgacggcggccagaaccuuccuccucuuccuccuccucgcccgcggcccccgcgcgcccgcggccgugccccgcggucccggccccggcccccggcgacacgcacuuccgcacauuccguucgcacgccgauuaccggcgcaucacgcgcgccagcgcgcuccuggacgccugcggauucuacugggggccccugagcgugcacggggcgcacgagcggcugcgcgccgagcccgugggcaccuuccuggugcgcgacagccgccagcggaacugcuuuuucgcccuuagcgugaagauggccucgggacccacgagcauccgcgugcacuuucaggccggccgcuuucaccuggauggcagccgcgagagcuucgacugccucuucgagcugcuggagcacuacguggcggcgccgcgccgcaugcugggggccccgcugcgccagcgccgcgugcggccgcugcaggagcugugccgccagcgcaucguggccaccgugggccgcgagaaccuggcucgcaucccccucaaccccguccuccgcgacuaccugagcuccuuccccuuccagauuugaccggcagcgcccgccgugcacgcagcauuaacugggaugccguguuauuuuguuauuacuugccuggaaccauguggguacccuccccggccuggguuggagggagcggauggguguaggggcgaggcgccucccgcccucggcuggagacgaggccgcagaccccuucucaccucuugaggggguccucccccuccuggugcucccucugggucccccugguuguuguagcagcuuaacuguaucuggagccaggaccugaacucgcaccuccuaccucuucauguuuacauauacccaguaucuuugcacaaaccagggguugggggagggucucuggcuuuauuuuucugcugugcagaauccuauuuuauauuuuuuaaagucaguuuagguaauaaacuuuauuaugaaaguuuuuuuuuu

SEQ ID NO: 18 - protein, 211 aa, Homo sapiens
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 19 - RNA, 1794 nt, Homo sapiens
auuucuuuauaaaccacaacucugggcccgcaauggcaguccacugccuugcugcagucacagaauggaaaucugcagaggccuccgcagucaccuaaucacucuccuccucuuccuguuccauucagagacgaucugccgacccucugggagaaaauccagcaagaugcaagccuucagaaucugggauguuaaccagaagaccuucuaucugaggaacaaccaacuaguugcuggauacuugcaaggaccaaaugucaauuuagaagaaaagauagaugugguacccauugagccucaugcucuguucuugggaauccauggagggaagaugugccuguccugugucaagucuggugaugagaccagacuccagcuggaggcaguuaacaucacugaccugagcgagaacagaaagcaggacaagcgcuucgccuucauccgcucagacaguggccccaccaccaguuuugagucugccgccugccccgguugguuccucugcacagcgauggaagcugaccagcccgucagccucaccaauaugccugacgaaggcgucauggucaccaaauucuacuuccaggaggacgaguaguacugcccaggccugccuguucccauucuugcauggcaaggacugcagggacugccagucccccugccccagggcucccggcuaugggggcacugaggaccagccauugagggguggacccucagaaggcgucacaacaaccuggucacaggacucugccuccucuucaacugaccagccuccaugcugccuccagaauggucuuucuaaugugugaaucagagcacagcagccccugcacaaagcccuuccaugucgccucugcauucaggaucaaaccccgaccaccugcccaaccugcucuccucuugccacugccucuuccucccucauuccaccuucccaugcccuggauccaucaggccacuugaugacccccaaccaaguggcucccacacccuguuuuacaaaaaagaaaagaccaguccaugagggagguuuuuaaggguuuguggaaaaugaaaauuaggauuucaugauuuuuuuuuuucaguccccgugaaggagagcccuucauuuggagauuauguucuuucggggagaggcugaggacuuaaaauauuccugcauuugugaaaugauggugaaaguaagugguagcuuuucccuucuuuuucuucuuuuuuugugaugucccaacuuguaaaaauuaaaaguuaugguacuauguuagccccauaauuuuuuuuuuccuuuuaaaacacuuccauaaucuggacuccucuguccaggcacugcugcccagccuccaagcuccaucuccacuccagauuuuuuacagcugccugcaguacuuuaccuccuaucagaaguuucucagcucccaaggcucugagcaaauguggcuccuggggguucuuucuuccucugcugaaggaauaaauugcuccuugacauuguagagcuucuggcacuuggagacuuguaugaaagauggcugugccucugccugucucccccaccgggcugggagcucugcagagcaggaaacaugacucguauaugucucaggucccugcagggccaagcaccuagccucgcucuuggcagguacucagcgaaugaaugcuguauauguugggugcaaaguucccuacuuccugugacuucagcucuguuuuacaauaaaaucuugaaaaugccuaaaaaaaaaaaaaaaaaaaaaaaaaaaaaaaaaaaaaaaaaaaaaaaaaaaa

SEQ ID NO: 20 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 21 - RNA, 4553 nt, Homo sapiens
acgcgucugcggccagcccggacucuuuaaaagccggcggugcgcggggcaucccagccaagccggagaggaggcgagcagcagggccugguggcgagagcgcggcugucacugcgcccgagcaucccagagcuuuccgagcggacgagccggccgugccgggcauccccagccucgcuacccucgcagcacacgucgagccccgcacaggcgaggguccggaacuuagcccaaagcacguuuccccuggcagcgcaggaaacgcccggccgcgcgccggcgcacgccccccucuccuccuuuguuccgggggucggcggccgcucuccugccagcgucgggaucucggccccgggaggcgggccgucgggcgcagccgcgaagaugccguuggaacugacgcagagccgagugcagaagaucugggugcccguggaccacaggcccucguugcccagauccugugggccaaagcugaccaacucccccaccgucaucgucauggugggccuccccgcccggggcaagaccuacaucuccaagaagcugacucgcuaccucaacuggauuggcguccccacaaaaguguucaacgucggggaguaucgccgggaggcugugaagcaguacagcuccuacaacuucuuccgccccgacaaugaggaagccaugaaaguccggaagcaaugugccuuagcugccuugagagaugucaaaagcuaccuggcgaaagaagggggacaaauugcgguuuucgaugccaccaauacuacuagagagaggagacacaugauccuucauuuugccaaagaaaaugacuuuaaggcguuuuucaucgagucggugugcgacgacccuacaguuguggccuccaauaucauggaaguuaaaaucuccagcccggauuacaaagacugcaacucggcagaagccauggacgacuucaugaagaggaucaguugcuaugaagccagcuaccagccccucgaccccgacaaaugcgacagggacuugucgcugaucaaggugauugacgugggccggagguuccuggugaaccgggugcaggaccacauccagagccgcaucguguacuaccugaugaacauccacgugcagccgcguaccaucuaccugugccggcacggcgagaacgagcacaaccuccagggccgcaucgggggcgacucaggccuguccagccggggcaagaaguuugccagugcucugagcaaguucguggaggagcagaaccugaaggaccugcgcguguggaccagccagcugaagagcaccauccagacggccgaggcgcugcggcugcccuacgagcaguggaaggcgcucaaugagaucgacgcgggcgucugugaggagcugaccuacgaggagaucagggacaccuacccugaggaguaugcgcugcgggagcaggacaaguacuauuaccgcuaccccaccggggaguccuaccaggaccugguccagcgcuuggagccagugaucauggagcuggagcggcaggagaaugugcuggucaucugccaccaggccguccugcgcugccugcuugccuacuuccuggauaagagugcagaggagaugcccuaccugaaaugcccucuucacaccguccugaaacugacgccugucgcuuauggcugccguguggaauccaucuaccugaacguggaguccgucugcacacaccgggagaggucagaggaugcaaagaagggaccuaacccgcucaugagacgcaauagugucaccccgcuagccagccccgaacccaccaaaaagccucgcaucaacagcuuugaggagcauguggccuccaccucggccgcccugcccagcugccugcccccggaggugcccacgcagcugccuggacaaaacaugaaaggcucccggagcagcgcugacuccuccaggaaacacugaggcagacgugucgguuccauuccauuuccauuucugcagcuuagcuuguguccugcccuccgcccgaggcaaaacguauccugaggacuucuuccggagagggugggguggagcagcgggggagccuuggccgaagagaaccaugcuuggcaccgucuguguccccucggccgcuggacaccagaaagccacgugggucccuggcgcccugccuuuagccguggggcccccaccuccacucucuggguuuccuaggaauguccagccucggagaccuucacaaagccuugggagggugaugagugcugguccugacaggaggccgcuggggacacugugcuguuuuguuucguuucugugaucucccggcacguuuggagcugggaagaccacacugguggcagaauccuaaaauuaaaggaggcaggcuccuaguugcugaaaguuaaggaauguguaaaaccuccacgugacuguuuggugcaucuugaccugggaagacgccucaugggaacgaacuuggacagguguuggguugaggccucuucugcaggaagucccugagcugagacgcaaguuggcugggugguccgcacccuggcucuccugcagguccacacaccuuccaggccuguggccugccuccaaagaugugcaagggcaggcuggcugcacggggagagggaaguauuuugccgaaauaugagaacuggggccuccugcucccagggagcuccagggccccucucuccucccaccuggacuuggggggaacugagaaacacuuuccuggagcugcuggcuuuugcacuuuuuugauggcagaagugugaccugagagucccaccuucucuucaggaacguagauguuggggugucuugcccuggggggcuuggaaccucugaagguggggagcggaacaccuggcauccuuccccagcacuugcauuaccgucccugcucuucccagguggggacaguggcccaagcaaggccucacucgcagccacuucuucaagagcugccugcacacugucuuggagcaucugccuugugccuggcacucugccggugccuugggaaggucggaagaguggacuuuguccuggccuucccuucauggcgucuaugacacuuuuguggugauggaaagcaugggaccugucgucucagccuguugguuucuccucauugccucaaacccugggguaggugggacggggggucucgugcccagaugaaaccauuuggaaacucggcagcagaguuuguccaaaugacccuuuucaggaugucucaaagcuugugccaaaggucacuuuucuuuccugccuucugcugugagcccugagauccuccucccagcucaagggacagguccugggugagggugggagauuuagacaccugaaacugggcguggagagaagagccguugcuguuuguuuuuugggaagagcuuuuaaagaaugcauguuuuuuuccugguuggaauugaguaggaacugaggcugugcuucagguaugguacaaucaagugggggauuuucaugcugaaccauucaagcccuccccgcccguugcacccacuuuggcuggcgucugcuggagaggaugucucuguccgcauucccgugcagcuccaggcucgcgcaguuuucucucucucccuggauguugagucucaucagaauauguggguaggggguggacgugcacgggugcaugauugugcuuaacuugguuguauuuuucgauuugacauggaaggccuguugcuuugcucuugagaauaguuucucgugucccccucgcaggccucauucuuugaacaucgacucugaaguuugauacagauaggggcuugauagcugugguccccucuccccucugacuaccuaaaaucaauaccuaaauacagaagccuuggucuaacacgggacuuuuaguuugcgaagggccuagauagggagagagguaacaugaaucuggacagggagggagauacuauagaaaggagaacacugccuacuuugcaagccagugaccugccuuuugaggggacauuggacgggggccgggggcggggguuggguuugagcuacagucaugaacuuuuggcgucuacugauuccuccaacucuccaccccacaaaauaacggggaccaauauuuuuaacuuugccuauuuguuuuugggugaguuuccccccuccuuauucuguccugagaccacgggcaaagcucuucauuuugagagagaagaaaaacuguuuggaaccacaccaaugauauuuuucuuuguaauacuugaaauuuauuuuuuuauuauuuugauagcagaugugcuauuuauuuauuuaauauguauaaggagccuaaacaauagaaagcuguagagauuggguuucauuguuaauugguuugggagccuccuaugugugacuuaugacuucucuguguucuguguauuugucugaauuaaugaccugggauauaaagcuaugcuagcuuucaaacaggagaugccuuucagaaauuuguauauuuugcaguugccagaccaauaaaauaccugguugaaauacauggacgaaguaaaaaa

SEQ ID NO: 22 - protein, 520 aa, Homo sapiens
MPLELTQSRVQKIWVPVDHRPSLPRSCGPKLTNSPTVIVMVGLPARGKTYISKKLTRYLNWIGVPTKVFNVGEYRREAVKQYSSYNFFRPDNEEAMKVRKQCALAALRDVKSYLAKEGGQIAVFDATNTTRERRHMILHFAKENDFKAFFIESVCDDPTVVASNIMEVKISSPDYKDCNSAEAMDDFMKRISCYEASYQPLDPDKCDRDLSLIKVIDVGRRFLVNRVQDHIQSRIVYYLMNIHVQPRTIYLCRHGENEHNLQGRIGGDSGLSSRGKKFASALSKFVEEQNLKDLRVWTSQLKSTIQTAEALRLPYEQWKALNEIDAGVCEELTYEEIRDTYPEEYALREQDKYYYRYPTGESYQDLVQRLEPVIMELERQENVLVICHQAVLRCLLAYFLDKSAEEMPYLKCPLHTVLKLTPVAYGCRVESIYLNVESVCTHRERSEDAKKGPNPLMRRNSVTPLASPEPTKKPRINSFEEHVASTSAALPSCLPPEVPTQLPGQNMKGSRSSADSSRKH

SEQ ID NO: 23 - RNA, 899 nt, Homo sapiens
agccuacgcacgaaagugacuaggaggaaggauauuauaaagugaugcaaacagaaauuccaccagccuccauguaucaucaugugucauaacucagucaagcucagugagcauucucagcacauugccucaacagcuucaaggugagccagcucaagacuuugcucuccaccaggcagaagaugacagacugugaauuuggauauauuuacaggcuggcucaggacuaucugcagugcguccuacagauaccacaaccuggaucagguccaagcaaaacguccagagugcuacaaaauguugcguucucaguccaaaaagaaguggaaaagaaucugaagucaugcuuggacaauguuaauguuguguccguagacacugccagaacacuauucaaccaagugauggaaaaggaguuugaagacggcaucauuaacuggggaagaauuguaaccauauuugcauuugaagguauucucaucaagaaacuucuacgacagcaaauugccccggauguggauaccuauaaggagauuucauauuuuguugcggaguucauaaugaauaacacaggagaauggauaaggcaaaacggaggcugggaaaauggcuuuguaaagaaguuugaaccuaaaucuggcuggaugacuuuucuagaaguuacaggaaagaucugugaaaugcuaucucuccugaagcaauacuguugaccagaaaggacacuccauauugugaaaccggccuaauuuuucugacugauauggaaacgauugccaacacauacuucuacuuuuaaauaaacaacuuugaugauguaacuugaccuuccagaguuauggaaauuuuguccccauguaaugaauaaauuguauguauuuuucucuauaaaaaaaaaaaaa

SEQ ID NO: 24 - protein, 175 aa, Homo sapiens
MTDCEFGYIYRLAQDYLQCVLQIPQPGSGPSKTSRVLQNVAFSVQKEVEKNLKSCLDNVNVVSVDTARTLFNQVMEKEFEDGIINWGRIVTIFAFEGILIKKLLRQQIAPDVDTYKEISYFVAEFIMNNTGEWIRQNGGWENGFVKKFEPKSGWMTFLEVTGKICEMLSLLKQYC

SEQ ID NO: 25 - RNA, 1227 nt, Homo sapiens
cuuugcagauaaauauggcacacuagccccacguuuucugagacauuccucaauugcuuagacauauucugagccuacagcagaggaaccuccagucucagcaccaugaaucaaacugccauucugauuugcugccuuaucuuucugacucuaaguggcauucaaggaguaccucucucuagaacuguacgcuguaccugcaucagcauuaguaaucaaccuguuaauccaaggucuuuagaaaaacuugaaauuauuccugcaagccaauuuuguccacguguugagaucauugcuacaaugaaaaagaagggugagaagagaugucugaauccagaaucgaaggccaucaagaauuuacugaaagcaguuagcaaggaaaggucuaaaagaucuccuuaaaaccagaggggagcaaaaucgaugcagugcuuccaaggauggaccacacagaggcugccucucccaucacuucccuacauggaguauaugucaagccauaauuguucuuaguuugcaguuacacuaaaaggugaccaaugauggucaccaaaucagcugcuacuacuccuguaggaagguuaauguucaucauccuaagcuauucaguaauaacucuacccuggcacuauaauguaagcucuacugaggugcuauguucuuaguggauguucugacccugcuucaaauauuucccucaccuuucccaucuuccaaggguacuaaggaaucuuucugcuuugggguuuaucagaauucucagaaucucaaauaacuaaaagguaugcaaucaaaucugcuuuuuaaagaaugcucuuuacuucauggacuuccacugccauccucccaaggggcccaaauucuuucaguggcuaccuacauacaauuccaaacacauacaggaagguagaaauaucugaaaauguauguguaaguauucuuauuuaaugaaagacuguacaaaguagaagucuuagauguauauauuuccuauauuguuuucaguguacauggaauaacauguaauuaaguacuauguaucaaugaguaacaggaaaauuuuaaaaauacagauagauauaugcucugcauguuacauaagauaaaugugcugaaugguuuucaaaauaaaaaugagguacucuccuggaaauauuaagaaagacuaucuaaauguugaaagaucaaaagguuaauaaaguaauuauaacuaagaaaaaaaaaaaa

SEQ ID NO: 26 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 27 - RNA, 1351 nt, Homo sapiens
gugauggagagcaccagcaaagccuuagggcccaucccuggccuccuguuacccacagagggguaggcccuuggcucucuuccacuaugacgucagcuuccauucuuccuuucuuauagacaauuuuccauuucaaggaaaucagagcccuuaauaguucagugaggucacuuugcugagcacaaucccauacccuucagccucugcuccacagagccuaagcaaaagauagaaacucacaacuuccuuguuuuguuaucuggaaauuaucccaggaucuggugcuuacucagcauauucaaggaaggucuuacuucauucuuccuugauugugaccaugcccaggcucucugcucccuauaaaaggcaggcagagccaccgaggagcagagagguugagaacaacccagaaaccuucaccucucaugcugaagcucacacccuugcccuccaagaugaagguuucugcagcgcuucugugccugcugcucauggcagccacuuucagcccucagggacuugcucagccagauucaguuuccauuccaaucaccugcugcuuuaacgugaucaauaggaaaauuccuauccagaggcuggagagcuacacaagaaucaccaacauccaaugucccaaggaagcugugaucuucaagaccaaacggggcaaggaggucugugcugaccccaaggagagaugggucagggauuccaugaagcaucuggaccaaauauuucaaaaucugaagccaugagccuucauacauggacugagagucagagcuugaagaaaagcuuauuuauuuuccccaaccucccccaggugcagugugacauuauuuuauuauaacauccacaaagagauuauuuuuaaauaauuuaaagcauaauauuucuuaaaaaguauuuaauuauauuuaaguuguugauguuuuaacucuaucugucauacauccuagugaauguaaaaugcaaaauccuggugauguguuuuuuguuuuuguuuuccugugagcucaacuaaguucacggcaaaaugucauuguucucccuccuaccugucuguaguguugugggguccucccauggaucaucaaggugaaacacuuugguauucuuuggcaaucagugcuccuguaagucaaaugugugcuuuguacugcuguuguugaaauugauguuacuguauauaacuauggaauuuugaaaaaaaauuucaaaaagaaaaaaauauauauaauuuaaaacuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - protein, 99 aa, Homo sapiens
MKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

SEQ ID NO: 29 - RNA, 1525 nt, Homo sapiens
cugcuugaagccuaacuguccaccagaaaggacugcucuuugggugaguugaacuucuuccauuauagaaagaauugaaggcugagaaacucagccucuaucauguggaacagcucugacgccaacuucuccugcuaccaugagucugugcugggcuaucguuauguugcaguuagcugggggguggugguggcugugacaggcaccgugggcaaugugcucacccuacuggccuuggccauccagcccaagcuccguacccgauucaaccugcucauagccaaccucacacuggcugaucuccucuacugcacgcuccuucagcccuucucuguggacaccuaccuccaccugcacuggcgcaccggugccaccuucugcaggguauuugggcuccuccuuuuugccuccaauucugucuccauccugacccucugccucaucgcacugggacgcuaccuccucauugcccacccuaagcuuuuuccccaaguuuucagugccaaggggauagugcuggcacuggugagcaccuggguugugggcguggccagcuuugcuccccucuggccuauuuauauccugguaccuguagucugcaccugcagcuuugaccgcauccgaggccggccuuacaccaccauccucaugggcaucuacuuugugcuugggcucagcaguguuggcaucuucuauugccucauccaccgccaggucaaacgagcagcacaggcacuggaccaauacaaguugcgacaggcaagcauccacuccaaccauguggccaggacugaugaggccaugccuggucguuuccaggagcuggacagcagguuagcaucaggaggacccagugaggggauuucaucugagccagucagugcugccaccacccagacccuggaaggggacucaucagaagugggagaccagaucaacagcaagagagcuaagcagauggcagagaaaagcccuccagaagcaucugccaaagcccagccaauuaaaggagccagaagagcuccggauucuucaucggaauuugggaaggugacucgaauguguuuugcuguguuccucugcuuugcccugagcuacauccccuucuugcugcucaacauucuggaugccagaguccaggcuccccgggugguccacaugcuugcugccaaccucaccuggcucaaugguugcaucaacccugugcucuaugcagccaugaaccgccaauuccgccaagcauauggcuccauuuuaaaaagagggccccggaguuuccauaggcuccauuagaacugugacccuagucaccagaauucaggacugucuccuccaggaccaaaguggccagguaauaggagaauaggugaaauaacacaugugggcauuuucacaacaaucucuccccagccucccaaaucaagucucuccaucacuugaucaauguuucagcccuagacugcccaaggaguauuauuaauuauuaauaaaugaauucugugcuuuuaaaaaaaaaaaaaaaaaa

SEQ ID NO: 30 - protein, 396 aa, Homo sapiens
MWNSSDANFSCYHESVLGYRYVAVSWGVVVAVTGTVGNVLTLLALAIQPKLRTRFNLLIANLTLADLLYCTLLQPFSVDTYLHLHWRTGATFCRVFGLLLFASNSVSILTLCLIALGRYLLIAHPKLFPQVFSAKGIVLALVSTWVVGVASFAPLWPIYILVPVVCTCSFDRIRGRPYTTILMGIYFVLGLSSVGIFYCLIHRQVKRAAQALDQYKLRQASIHSNHVARTDEAMPGRFQELDSRLASGGPSEGISSEPVSAATTQTLEGDSSEVGDQINSKRAKQMAEKSPPEASAKAQPIKGARRAPDSSSEFGKVTRMCFAVFLCFALSYIPFLLLNILDARVQAPRVVHMLAANLTWLNGCINPVLYAAMNRQFRQAYGSILKRGPRSFHRLH

SEQ ID NO: 31 - RNA, 839 nt, Homo sapiens
gcgugaccagagcgcgcuggcccggcccacccggggcgguuguggucgcuauauauaagguggggaggccgccggcccguucgguuccgggcguuaccaucguccgugcgcaccgcccggcguccagauuuggcaauucuucgcugaagucaucaugagcuuuuuccaacuccugaugaaaaggaaggaacucauucccuuggugguguucaugacuguggcggcggguggagccucaucuuucgcuguguauucucuuuggaaaaccgaugugauccuugaucgaaaaaaaaauccagaaccuugggaaacuguggacccuacuguaccucaaaagcuuauaacaaucaaccaacaauggaaacccauugaagaguugcaaaauguccaaagggugaccaaaugacgagcccucgccucuuucuucugaagaguacucuauaaaucuaguggaaacauuucugcacaaacuagauucuggacaccagugugcggaaaugcuucugcuacauuuuuaggguuugucuacauuuuuugggcucuggauaaggaauuaaaggagugcagcaauaacugcacugucuaaaaguuugugcuuauuuucuuguaaauuugaauauugcauauugaaauuuuuguuuaugaucuaugaauguuuuucuuaaaauuuacaaagcuuuguaaauuagauuuucuuuaauaaaaugccauuugugcaagauuucucaaagauuagguauauauuuaaauggaagagaaaauauuuuuaugggagaaaaauacauuugaaccaugaaauuucaucuuuuaaauaacauccaguacagauuucuguguaa

SEQ ID NO: 32 - protein, 83 aa, Homo sapiens
MSFFQLLMKRKELIPLVVFMTVAAGGASSFAVYSLWKTDVILDRKKNPEPWETVDPTVPQKLITINQQWKPIEELQNVQRVTK

SEQ ID NO: 33 - RNA, 3553 nt, Homo sapiens
gagccagcgcgguagggccagagugggaaggccagagcgggcguccccgccagugaccccacgccgcccguccgcgcccaacccggccuccgccgaguguccaaaccaaaagcgaaaggaacccgaccccgcguccccuccggcggcuccguagucgcguccgcuuggagcucgccgggcgccuccgacccugccgggccgcuuugugacuucacucguuucgcaacaagcccgggcagcccgcgccccacccacucuggcccggcagccucgccgcccgcagccucgcuccgcuccucgcgcuuccccucccuccggggcugggccugccccggccgucgcggagccuccccucccaccguccgugaguguacgcgcccggccgccgccuccaggcagcccggagcaacccggcgcccggccccgcugggcgcagcacuccgucggcggcggcggcggcgcgaugcugugcuuccugaggggaauggcuuucguccccuuccucuuggugaccuggucgucagccgccuucauuaucuccuacguggucgccgugcucuccgggcacgucaaccccuuccucccguauaucagugauacgggaacaacaccuccagagagugguauuuuuggauuuaugauaaacuucucugcauuucuuggugcagccacgauguauacaagauacaaaauaguacagaagcaaaaucaaaccugcuauuucagcacuccuguuuuuaacuuggugucuuuagugcuuggauuggugggauguuucggaaugggcauugucgccaauuuucaggaguuagcugugccagugguucaugacgggggcgcucuuuuggccuuugucuguggugucguguacacgcuccuacaguccaucaucucuuacaaaucauguccccaguggaacagucucucgacaugccacauacggauggucaucucugccguuucuugcgcagcugucauccccaugauugucugugcuucacuaauuuccauaaccaagcuggaguggaauccaagagaaaaggauuauguauaucacguagugagugcgaucugugaauggacaguggccuuugguuuuauuuucuacuuccuaacuuucauccaagauuuccagagugucacccuaaggauauccacagaaaucaauggugauauuugaagaaagaagaauucagucucacucagugaaugucgcaggccauuucuaaaagugcuacagaggacagacaggguuuugaggccacccugauuauugggaugcaucugcagcacauccaggacuugaauuucauuacgaguuccuaauaguuguauuucuaaagauguguuuccuagagaauguacagccuuaugacacuguagugauguuuuuauaauuuucuaaguagauuuuuuuauauuaacaaauucauauacagaaaaaauaagguguuacaaaaaauggagagcucuuauuuuuguacagauucugucguuuuuguuuuauuugugugagauuuauggaaauacacuaaaugaguaauucagguucaguacauuuauuacaaagugaaaucaggggauauucauuuguaaauuuuauucuuagugaaugaacuguauaauuuuuuuuaucaggagagcacuuauaaaauucaauuuauaaagaucauauacccaaaucauaaagauuuaguugauacauuaacacuaagauacucugauuuuuagccgaacuaaacaaagugcuucuacugagaggccuuuauaccaccauguacaguaacucuaagugaauacggaagaccuugguuuugaaauucugccaccuuguuucucccugcucaugaggucgcaccuuuugcucuugcugcuaauugcccauucguaguggguguaaugccagguggaaugguuucaacaagucaggugaaaaccauccuuuauuguugcuggcacaacuugauauauagucugacucagaacugaagcucacaucucaaauucauuucaugccaguaaauguggcaaagagaagaaaggcccaagagcgagacaagaagaauggagaagggggcagccaagaagaacuucuggguucaggguacuguuuauuugcuccuucucuucaugccuguggcuggaugucccacaacacuauaagaaauauaagucaagcccuuuguguuaagcaagaacuacagacuccaucuuuucacccaaaucaugaaugaccaauaaaaagcaaguuauuccagaggaagaagcagcccuugaaauguuaaggcuuaggcuugaaaggugaagagcaggaauucucucuuucaaauccuagagcauaaacccauguguggccaagugagaucagcccucaagggcacaugccaagggcagagcagcccauguagacagcuucggagggcauggggguguagggaguucgggguagcuccucauuaacuauuuguugggugaguaaaggggugaggcucaguggcagguaccucugcaaugacaagcugccuccccucuauguguuuagcauauguuauuagaacauguccgacaccccuaccgcugccauuugggcccuuuaauaaagccaaguagagaaaucuggcaauaaaaggcaaauguaagcaugcuuucuuuaagacgcaucauaaaugguuuucuuuaagugaauggaagaguuugacagagauacaccuuuguaagaaaacauuaagaaugcuggcuggcugugguggcucacaccuguauucccagcacuuugggaggccuaggcaggaggauugcuugagccugggacuucgagaccagacugggaaacauggcaaaaucccaucucuacaacaaaaauacaaaaauuagccaagugcgguggugugccuguaguccuaguuacuugggaggcugaggugggagaaucaccugagcccaggagguggaggcugcagugagccaugccaaugcacuccagucugggcaacagagugagacccugucucaaaaauaaauaaauaaauaaaugaauaaagagaaugcuaaucauuucuggguucacugcgacucacuguagugcuggggaucccccuuguaacacuggaacugaaagacagugaugaaagcuaugucaagcauucauuauucugaagaggaggagaaaugccacauaccuuucccaugggaccugugguggaaugaauccauacuucugccucacuucgagcagacuuuuguucucggcgcuccucacgauggaguuucaugcuucauuuucacaucucucugcacaauuagauugggagcuccuugagggcagaguacgugccuuaaucuuuaucuuuguaaugccacaaugaacagagugccuccugguacacuguaggagcuuaagaaauacucacugaaugcaugaaugaaugaaugaacaaaugaaggaaugacuaaggauguuuguagugcuauaauauagaaugggauuuacucugcuuuaccaguuaguuucauaauaaacaaauagucuguaucgccugguaaaaaaaaaaaa

SEQ ID NO: 34 - protein, 238 aa, Homo sapiens
MLCFLRGMAFVPFLLVTWSSAAFIISYVVAVLSGHVNPFLPYISDTGTTPPESGIFGFMINFSAFLGAATMYTRYKIVQKQNQTCYFSTPVFNLVSLVLGLVGCFGMGIVANFQELAVPVVHDGGALLAFVCGVVYTLLQSIISYKSCPQWNSLSTCHIRMVISAVSCAAVIPMIVCASLISITKLEWNPREKDYVYHVVSAICEWTVAFGFIFYFLTFIQDFQSVTLRISTEINGDI

SEQ ID NO: 35 - RNA, 1610 nt, Homo sapiens
agagaacaaaacagaaacucuuggaagcaggaaaggugcaugacucaaagagggaaauuccugugccauaaaaggauugcugguguauaaaaugcucuauauaugccaauuaucaauuuccuuucauguucagcauuucuacuccuuccaagaagagcagcaaagcugaaguagcagcagcagcaccagcagcaacagcaaaaaacaaacaugagugugaagggcauggcuauagccuuggcugugauauugugugcuacaguuguucaaggcuuccccauguucaaaagaggacgcugucuuugcauaggcccugggguaaaagcagugaaaguggcagauauugagaaagccuccauaauguacccaaguaacaacugugacaaaauagaagugauuauuacccugaaagaaaauaaaggacaacgaugccuaaaucccaaaucgaagcaagcaaggcuuauaaucaaaaaaguugaaagaaagaauuuuuaaaaauaucaaaacauaugaaguccuggaaaagagcaucugaaaaaccuagaacaaguuuaacugugacuacugaaaugacaagaauucuacaguaggaaacugagacuuuucuaugguuuugugacuuucaacuuuuguacaguuaugugaaggaugaaaggugggugaaaggaccaaaaacagaaauacagucuuccugaaugaaugacaaucagaauuccacugcccaaaggaguccaacaauuaaauggauuucuaggaaaagcuaccuuaagaaaggcugguuaccaucggaguuuacaaagugcuuucacguucuuacuuguugcauuauacauucaugcauuucuaggcuagagaaccuucuagauuugaugcuuacaacuauucuguugugacuaugagaacauuucugucucuagaagucaucugucuguauugaucuuuaugcuauauuacuaucugugguuacgguggagacauugacauuauuacuggagucaagcccuuauaagucaaaagcaucuaugugucguaaaacauuccucaaacauuuuuucaugcaaauacacacuucuuuccccaaacaucauguagcacaucaauauguagggagacauucuuaugcaucauuugguuuguuuuauaaccaauucauuaaauguaauucauaaaauguacuaugaaaaaaauuauacgcuaugggauacuggcaaaagugcacauauuucauaaccaaauuaguagcaccagucuuaauuugauguuuuucaacuuuuauucauugagauguuuugaagcaauuaggauauguguguuuacuguacuuuuuguuuugauccguuuguauaaaugauagcaauaucuuggacacaucugaaauacaaaauguuuuugucuaccaaagaaaaauguugaaaaauaagcaaauguauaccuagcaaucacuuuuacuuuuuguaauucugucucuuagaaaaauacauaaucuaaucaauuucuuuguucaugccuauauacuguaaaauuuagguauacucaagacuaguuuaaagaaucaaagucauuuuuuucucuaauaaacuaccacaaccuuucuuuuuuaaaaaaaaaaa

SEQ ID NO: 36 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 37 - RNA, 1439 nt, Homo sapiens
agucacauuucagccacugcucugagaauuugugagcagccccuaacaggcuguuacuucacuacaacugacgauaugaucaucuuaauuuacuuauuucucuugcuaugggaagacacucaaggauggggauucaaggauggaauuuuucauaacuccauauggcuugaacgagcagccgguguguaccacagagaagcacggucuggcaaauacaagcucaccuacgcagaagcuaaggcggugugugaauuugaaggcggccaucucgcaacuuacaagcagcuagaggcagccagaaaaauuggauuucaugucugugcugcuggauggauggcuaagggcagaguuggauaccccauugugaagccagggcccaacuguggauuuggaaaaacuggcauuauugauuauggaauccgucucaauaggagugaaagaugggaugccuauugcuacaacccacacgcaaaggagugugguggcgucuuuacagauccaaagcaaauuuuuaaaucuccaggcuucccaaaugaguacgaagauaaccaaaucugcuacuggcacauuagacucaaguauggucagcguauucaccugaguuuuuuagauuuugaccuugaagaugacccagguugcuuggcugauuauguugaaauauaugacaguuacgaugauguccauggcuuugugggaagauacuguggagaugagcuuccagaugacaucaucaguacaggaaaugucaugaccuugaaguuucuaagugaugcuucagugacagcuggagguuuccaaaucaaauauguugcaauggauccuguauccaaauccagucaaggaaaaaauacaaguacuacuucuacuggaaauaaaaacuuuuuagcuggaagauuuagccacuuauaaaaaaaaaaaaaaggaugaucaaaacacacaguguuuauguuggaaucuuuuggaacuccuuugaucucacuguuauuauuaacauuuauuuauuauuuuucuaaaugugaaagcaauacauaauuuagggaaaauuggaaaauauaggaaacuuuaaacgagaaaaugaaaccucucauaaucccacugcauagaaauaacaagcguuaacauuuucauauuuuuuucuuucagucauuuuucuauuugugguauauguauauauguaccuauauguauuugcauuugaaauuuuggaauccugcucuauguacaguuuuguauuauacuuuuuaaaucuugaacuuuauaaacauuuucugaaaucauugauuauucuacaaaaacaugauuuuaaacagcuguaaaauauucuaugauaugaauguuuuaugcauuauuuaagccugucucuauuguuggaauuucaggucauuuucauaaauauuguugcaauaaauauccuugaacacacaaaaaaaaaaaaaaaa

SEQ ID NO: 38 - protein, 277 aa, Homo sapiens
MIILIYLFLLLWEDTQGWGFKDGIFHNSIWLERAAGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKECGGVFTDPKQIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL

SEQ ID NO: 39 - RNA, 3188 nt, Homo sapiens
gggcggggcugggcgcaggcagucucccgccgccgccgccgcugccggacgcgcagagcgaggggcggcuggaccgacggcugccgggccgagcgcacagagucgcggcgcagggggcguccccggccgggacgcgggucgcgucguuguccuccgcgagcguccggauugcaggcugucuguccccagaccccagagcacguccggcaccaccaugacugggcuguugaagaggaaauuugaccagcuggaugaggacaacuccucggucuccuccuccuccucuuccucugggugccagucucgcuccugcuccccaagcucuucugucucccgugccugggacucagaggaggaaggccccugggaucagaugccccugccugaccgugacuucugcggccccagaaguuucaccccccugucuauccugaagcgggcucgccgggagcgcccaggccguguagccuuugaugggaucaccgucuucuacuucccccgcugccagggcuucaccagugugcccagccgugguggcuguacucuggguauggcccuucgccacagugcuugccgucgcuucucuuuggcugaguuugcgcaggagcaagcccgugcacggcacgagaagcuccgccagcgcuugaaagaggagaaguuggagaugcugcaguggaagcuuucggcagcugggguaccccaggcagaggcagggcugccaccugugguggaugccauugaugacgccucuguggaggaggacuuggcagucgcuguggcagguggccgguuggaagaagugagcuuccuacagcccuacccagcccggcgacgucgagcucugcugagggcuucaggugugcgaaggaucgaucgggaggagaagcgggagcugcaggcacugcgccaaucccgggaggauuguggcugucacugcgauaggaucugcgacccugagaccugcagcugcagccuggcaggcaucaagugccagauggaccacacagcauuccccuguggcugcugcagggagggcugugagaaccccaugggccguguggaauuuaaucaggcaagaguucagacccauuucauccacacacucacccgccugcaguuggaacaggaggcugagagcuuuagggagcuggaggccccugcccagggcagcccacccagcccuggugaggaggcccuggucccuacuuucccacuggccaagccccccaugaacaaugagcugggagacaacagcugcagcagcgacaugacugauucuucuacagcaucuucaucagcaucgggcacuagugaggcuccugacugccccacccacccaggccugccuggcccuggcuuccagccuggcguugaugaugacagccuggcacgcaucuugaguuucagugacucugacuucgguggggaggaggaggaagaggaggaagggagcguggggaaccuggacaaccucagcugcuuccauccagcugacaucuuugguacuagugacccugguggccuggccagcuggacccacagcuauucuggcuguagcuucacaucaggcguccuggaugagaaugccaaccuggaugccagcugcuuccuaaaugguggccuugaagggucaagggaaggcagccuuccuggcaccucagugccacccagcauggacgcuggccggaguagcucaguggaucucagcuugucuucuugugacuccuuugaguuacuccaggcucugccagauuauagucuggggccucacuacacaucacagaaggugucugacagccuggacaacaucgaggcaccucacuucccccugccuggccugucuccaccuggggaugccagcaguugcuuccuggagucccucaugggcuucuccgagccagccgccgaagcccuagaucccuuuauugacagccaguuugaggacacugucccagcaucucuaauggagccugugccggugugaggaccaggaugucuuuucccagccccaagagaccuguugcugcuuucuuguaauuauggggcuccccagagucugcguaacagucucccacuggcuggcucacccacaggugccaugugcacacuccugguuuucaaacaauucucuggauuuauuuauuuguuuuaacuuuucugugcugaagagaggacuggggggagggggcuuccccuuucagcugcccggccccccacacccacagcuugcucuucuaucuccacaacgugagccuggaagaggagaaaauguggcuccucuggagcuuggcagaccacuuuucggucuuugcgugauguuccuuagcccaaagacggugagacagggcugaaaucagguggcuucugccacccugagcccuagacccauggguggcuaaauccacuggacugugaagacuauaauuuauuuccauaauuuauuuggagauugaggaggcuuugguugcacuucuuuggcugguggguaaugccagggguggggugggcacaggcccucaagagccccuuuugccuuguaguccuacaccuugcccugccugggcuuuggugcagacuagguguggauuugagcucugugaucuaugucugcugccuggcuccuagauggcucugugggcaggugcuggccaaggacaucaucuaggcagggggagagccugggcugaacagcugugaccaaaacucccuucugccccacccugcccccuccacuuccugcccucuguuccaucuucccccuucccaaaggccacagccuuuauuccaggcccagggauguaggagggggaaggaggaaacaggaagcccagagagggcaaagggccuaccucggggcgcgaaccaugccccagacuauuaucucagggcuuucugggcacugcacuucagcguggcccaccugcccaugcccugaggccaguuggcgagggguggcuccugaggguuuuuauacccuuuguuugcuaauguuuaauuuugcaucauaauuucuacauugucccugagugucagaacuauaauuuauuccauuucucucugugucugugccaagaaacgcaggcucugggccugccccuugcccaggaggccuugccagccugugugcuugugggaacaccuuguaccugagcuuacagguaccaauaaagaggcuuuauuuuuagcaa

SEQ ID NO: 40 - protein, 589 aa, Homo sapiens
MTGLLKRKFDQLDEDNSSVSSSSSSSGCQSRSCSPSSSVSRAWDSEEEGPWDQMPLPDRDFCGPRSFTPLSILKRARRERPGRVAFDGITVFYFPRCQGFTSVPSRGGCTLGMALRHSACRRFSLAEFAQEQARARHEKLRQRLKEEKLEMLQWKLSAAGVPQAEAGLPPVVDAIDDASVEEDLAVAVAGGRLEEVSFLQPYPARRRRALLRASGVRRIDREEKRELQALRQSREDCGCHCDRICDPETCSCSLAGIKCQMDHTAFPCGCCREGCENPMGRVEFNQARVQTHFIHTLTRLQLEQEAESFRELEAPAQGSPPSPGEEALVPTFPLAKPPMNNELGDNSCSSDMTDSSTASSSASGTSEAPDCPTHPGLPGPGFQPGVDDDSLARILSFSDSDFGGEEEEEEEGSVGNLDNLSCFHPADIFGTSDPGGLASWTHSYSGCSFTSGVLDENANLDASCFLNGGLEGSREGSLPGTSVPPSMDAGRSSSVDLSLSSCDSFELLQALPDYSLGPHYTSQKVSDSLDNIEAPHFPLPGLSPPGDASSCFLESLMGFSEPAAEALDPFIDSQFEDTVPASLMEPVPV

SEQ ID NO: 41 - RNA, 2228 nt, Homo sapiens
caccggacaaacgucucuggagucucuccaaugagcaagaaagcaagucggggguaggggaggggccucacaccagggggugggcgcagucccuccuccagcuccuucacccuccaguagucucguggguccccgagcgccagcgcgggaaccgggaaaaggaaaccguguuguguacguaagauucaggaaacgaaaccaggagccgcggguguuggcgcaaagguuacucccagacccuuuuccggcugacuucugagaagguugcgcagcagcugugcccggcagucuagaggcgcagaagaggaagccaucgccuggccccggcucucuggaccuugucucgcucgggagcggaaacagcggcagccagagaacuguuuuaaucauggacaaacaaaacucacagaugaaugcuucucacccggaaacaaacuugccaguuggguauccuccucaguauccaccgacagcauuccaaggaccuccaggauauaguggcuacccugggccccaggucagcuacccacccccaccagccggccauucagguccuggcccagcuggcuuuccugucccaaaucagccaguguauaaucagccaguauauaaucagccaguuggagcugcagggguaccauggaugccagcgccacagccuccauuaaacuguccaccuggauuagaauauuuaagucagauagaucagauacugauucaucagcaaauugaacuucuggaaguuuuaacagguuuugaaacuaauaacaaauaugaaauuaagaacagcuuuggacagaggguuuacuuugcagcggaagauacugauugcuguacccgaaauugcugugggccaucuagaccuuuuaccuugaggauuauugauaauaugggucaagaagucauaacucuggagagaccacuaagauguagcagcuguuguugucccugcugccuucaggagauagaaauccaagcuccuccugguguaccaauagguuauguuauucagacuuggcacccaugucuaccaaaguuuacaauucaaaaugagaaaagagaggauguacuaaaaauaagugguccauguguugugugcagcuguuguggagauguugauuuugagauuaaaucucuugaugaacagugugugguuggcaaaauuuccaagcacuggacuggaauuuugagagaggcauuuacagacgcugauaacuuuggaauccaguucccuuuagaccuugauguuaaaaugaaagcuguaaugauuggugccuguuuccucauugacuucauguuuuuugaaagcacuggcagccaggaacaaaaaucaggagugugguaguggauuagugaaagucuccucaggaaaucugaagucuguauauugauugagacuaucuaaacucauaccuguaugaauuaagcuguaaggccuguagcucugguuguauacuuuugcuuuucaaauuauaguuuaucuucuguauaacugauuuauaaagguuuuuguacauuuuuuaauacucauugucaauuugagaaaaaggacauaugaguuuuugcauuuauuaaugaaacuuccuuugaaaaacugcuuugaauuaugaucucugauucauuguccauuuuacuaccaaauauuaacuaaggccuuauuaauuuuuauauaaauuauaucuuguccuauuaaaucuaguuacaauuuauuucaugcauaagagcuaauguuauuuugcaaaugccauauauucaaaaaagcucaaagauaauuuucuuuacuauuauguucaaauaauauucaauaugcauauuaucuuuaaaaaguuaaauguuuuuuuaaucuucaagaaaucaugcuacacuuaacuucuccuagaagcuaaucuauaccauaauauuuucauauucacaagauauuaaauuaccaauuuucaaauuauuguuaguaaagaacaaaaugauucucucccaaagaaagacacauuuuaaauacuccuucacucuaaaacucugguauuauaacuuuugaaaguuaauauuucuacaugaaauguuuagcucuuacacucuauccuuccuagaaaaugguaauugagauuacucagauauuaauuaaauacaauaucauauauauauucacagaguauaaaccuaaauaaugaucuauuagauucaaauauuugaaauaaaaacuugauuuuuuuguaaaaaaaaaaaaaaaaaaa

SEQ ID NO: 42 - protein, 318 aa, Homo sapiens
MDKQNSQMNASHPETNLPVGYPPQYPPTAFQGPPGYSGYPGPQVSYPPPPAGHSGPGPAGFPVPNQPVYNQPVYNQPVGAAGVPWMPAPQPPLNCPPGLEYLSQIDQILIHQQIELLEVLTGFETNNKYEIKNSFGQRVYFAAEDTDCCTRNCCGPSRPFTLRIIDNMGQEVITLERPLRCSSCCCPCCLQEIEIQAPPGVPIGYVIQTWHPCLPKFTIQNEKREDVLKISGPCVVCSCCGDVDFEIKSLDEQCVVGKISKHWTGILREAFTDADNFGIQFPLDLDVKMKAVMIGACFLIDFMFFESTGSQEQKSGVW

SEQ ID NO: 43 - RNA, 2158 nt, Homo sapiens
auauucuacaucuaucggagcugaacuuccuaaaagacaaaguguuuaucuuucaagauucauucucccugaaucuuaccaacaaaacacuccugaggagaaagaaagagagggagggagagaaaaagagagagagagaaacaaaaaaccaaagagagagaaaaaaugaauucaucuaaaucaucugaaacacaaugcacagagagaggaugcuucucuucccaaauguucuuauggacuguugcugggauccccauccuauuucucagugccuguuucaucaccagauguguugugacauuucgcaucuuucaaaccugugaugagaaaaaguuucagcuaccugagaauuucacagagcucuccugcuacaauuauggaucagguucagucaagaauuguuguccauugaacugggaauauuuucaauccagcugcuacuucuuuucuacugacaccauuuccugggcguuaaguuuaaagaacugcucagccaugggggcucaccuggugguuaucaacucacaggaggagcaggaauuccuuuccuacaagaaaccuaaaaugagagaguuuuuuauuggacugucagaccagguugucgagggucaguggcaauggguggacggcacaccuuugacaaagucucugagcuucugggauguaggggagcccaacaacauagcuacccuggaggacugugccaccaugagagacucuucaaacccaaggcaaaauuggaaugauguaaccuguuuccucaauuauuuucggauuugugaaaugguaggaauaaauccuuugaacaaaggaaaaucucuuuaagaacagaaggcacaacucaaauguguaaagaaggaagagcaagaacauggccacacccaccgccccacacgagaaauuugugcgcugaacuucaaaggacuucauaaguauuuguuacucugauauaaauaaaaauaaguaguuuuaaauguuauaauucauguuacuggcugaagugcauuuucucucuacguuagucucagguccucuucccagaauuuacaaagcaauucacuaccuuuugcuacauuugccucauuuuuuaguguucguaugaaaguacagggacacggagccaagacagagucuagcaaagaaggggauuuuggaaggugccuuccaaaaaucuccugaauccgggcucuguagcagguccucuucuuucuagcuucugacaagucugucuucucuucuugguuucauaccguucuuaucuccugcccaagcauauaucgucucuuuacuccccuguauaaugaguaagaagcuucuucaagucaugaaacuuauuccugcucagaauaccgguguggccuuucuggcuacaggccuccacugcaccuucuuagggaagggcaugccagccaucagcuccaaacaggcuguaaccaaguccacccaucccuggggcuuccuuugcucugccuuauuuucaauugacugaauggaucucaccagauuuuguaucuauugcucagcuaggacccgaguccaauagucaauuuauucuaagcgaacauucaucuccacacuuuccugucucaagcccauccauuauuucuuaacuuuuauuuuagcuuucggggguacauguuaaaggcuuuuuauauagguaaacucaugucguggagguuuguuguacagauuauuucaucacccagguauuaagcccagugccuaauauuguuuuuuucggcuccucucccuccuccuaccuuccgcccucaaguagacuccagugucuguuauucccuucuuuguguuuaugaauucucaucauuuagcucccacuuauaagugaggacaugcaguauuugguuuucuguucccauguuugcuaaggauaauggucuccaguucuaccgauguucccacaaaagacauaaucuucuuuuuuaaggcugcuuaguauuccaugguaucuauguaucacauuuucucuauccaaucuauuguugacucacauuuagauugauuccauguuuuugcuauugugaauagugcugcaaugaacauucgugugcaugugucuuuaugguagaaagauuuauauuucucugaguauguauccaguaauagcccauucauuuauugcauaaaauucuaccaauac

SEQ ID NO: 44 - protein, 219 aa, Homo sapiens
MNSSKSSETQCTERGCFSSQMFLWTVAGIPILFLSACFITRCVVTFRIFQTCDEKKFQLPENFTELSCYNYGSGSVKNCCPLNWEYFQSSCYFFSTDTISWALSLKNCSAMGAHLVVINSQEEQEFLSYKKPKMREFFIGLSDQVVEGQWQWVDGTPLTKSLSFWDVGEPNNIATLEDCATMRDSSNPRQNWNDVTCFLNYFRICEMVGINPLNKGKSL

SEQ ID NO: 45 - RNA, 1922 nt, Homo sapiens
cuuaucugaugcugugcauuagacagcacacugcugacuguuuucaguuguuucuguaacagcagaaagugcacucacuaggaguagucagaauucaaaaugcucaagagaaagccauccaauguuucagagaaggagaaacaucaaaaaccaaagcgaagcagcaguuuugggaauuucgaucguuuucggaauaauucuuuaucaaaaccagaugauucaacugaggcacaugaaggagaucccacaaauggaaguggagaacaaaguaaaacuucaaauaauggaggcgguuuggguaaaaaaaugagagcuauuucauggacaaugaagaaaaaaguggguaaaaaguacaucaaagcccuuucugaggaaaaggaugaggaagauggagagaaugcccacccauauagaaacagugacccugugauugggacccacacagagaaggugucccucaaagccagugacuccauggauagucucuacaguggacagagcucaucaaguggcauaacaagcuguucagaugguacaaguaaccgggacagcuuucgacuggaugacgauggccccuauucaggaccauucuguggccgugccagagugcauacggauuucacgccaagucccuaugacacugacucccucaaaaucaagaaaggagacaucauagacauuauuugcaaaacaccaauggggauguggacaggaauguugaacaauaaagugggaaacuucaaauucauuuauguggaugucaucucagaagaggaagcagcccccaagaaaauaaaggcaaaccgaaggaguaacagcaaaaaauccaagacucugcaggaguuccuagagaggauucaucugcaggaauacaccucaacacuuuugcucaaugguuaugagacucuagaagauuuaaaagauauaaaagagagucaccucauugaauuaaauauugaaaacccagaugacagaagaagguuacuaucagcugcugaaaacuuccuugaagaagaaauuauucaagagcaagaaaaugaaccugagccccuauccuugagcucagacaucuccuuaaauaagucacaguuagaugacugcccaagggacucugguugcuauaucucaucaggaaauucagauaauggcaaagaggaucuggagucugaaaaucugucugacaugguacauaagauuauuaucacagagccaagugacugaacacgcauucccaacuauauaucuacagaugcauuccauuuuaacucuucuugagcuaaaacgucaaauaggagaggaagauaagauaaauauuuguaaauaaaaccuaaaguuuaaauguuuuaaucugaauaauuguacauaaaauuuuguaucucuaacauuccaaauuacugucaauaaaauauauauuuauuauuuuaaaugcuauguguuaauauuucacuugcuuguauuagaaaggcaaaauguaagacuuugguaugugugacauaugcuuuauuuggcuuuauuuuacaaguacaguaucugcaaaaaacaaaguaaccuuuuuucauaccugccaguuuugaauuuauauauguuauugaacaaauaguaauagaggauucgcuguugaaacaaguuguccaagcaauguuauauucauuuuuauacuuauugggaaagugugaguuaauauuggacacauuuuauccugauccacaguggaguuuuaguaauuauauuuuguugauuucuucauuuuguuuucugguauaaaaguagagauaauguguagucacuucugauuuagugaaaccaauuguaauaauuguggaaauguuuugucuuuaaguguaaauauuuuaaaauuugacauacccuaauguuaauaauaaaaagaacuauuugcauauugcaaaaaaaaaaaaaaaaaa

SEQ ID NO: 46 - protein, 373 aa, Homo sapiens
MLKRKPSNVSEKEKHQKPKRSSSFGNFDRFRNNSLSKPDDSTEAHEGDPTNGSGEQSKTSNNGGGLGKKMRAISWTMKKKVGKKYIKALSEEKDEEDGENAHPYRNSDPVIGTHTEKVSLKASDSMDSLYSGQSSSSGITSCSDGTSNRDSFRLDDDGPYSGPFCGRARVHTDFTPSPYDTDSLKIKKGDIIDIICKTPMGMWTGMLNNKVGNFKFIYVDVISEEEAAPKKIKANRRSNSKKSKTLQEFLERIHLQEYTSTLLLNGYETLEDLKDIKESHLIELNIENPDDRRRLLSAAENFLEEEIIQEQENEPEPLSLSSDISLNKSQLDDCPRDSGCYISSGNSDNGKEDLESENLSDMVHKIIITEPSD

SEQ ID NO: 47 - RNA, 3776 nt, Homo sapiens
gggacuguguuaagaaccaagggcauauaaagacagaugggaggagacccuggccggggagaggaagaagcaucuacauagguacuaaguaccauucgcuaauucagcuuagagaacuaucaacacaggacaaugcaagcccaugagcuguuccgguauuuucgaaugccagagcugguugacuuccgacaguacgugcguacucuuccgaccaacacgcuuaugggcuucggagcuuuugcagcacucaccaccuucugguacgccacgagacccaaaccccugaagccgccaugcgaccucuccaugcagucaguggaaguggcggguagugguggugcacgaagauccgcacuacuugacagcgacgagcccuugguguauuucuaugaugaugucacaacauuauacgaagguuuccagaggggaauacaggugucaaauaauggcccuuguuuaggcucucggaaaccagaccaacccuaugaauggcuuucauauaaacagguugcagaauugucggagugcauaggcucagcacugauccagaagggcuucaagacugccccagaucaguucauuggcaucuuugcucaaaauagaccugagugggugauuauugaacaaggaugcuuugcuuauucgauggugaucguuccacuuuaugauacccuuggaaaugaagccaucacguacauagucaacaaagcugaacucucucugguuuuuguugacaagccagagaaggccaaacucuuauuagaggguguagaaaauaaguuaauaccaggccuuaaaaucauaguugucauggaugccuacggcagugaacugguggaacgaggccagaggugugggguggaagucaccagcaugaaggcgauggaggaccugggaagagccaacagacggaagcccaagccuccagcaccugaagaucuugcaguaauuuguuucacaaguggaacuacaggcaaccccaaaggagcaauggucacucaccgaaacauagugagcgauuguucagcuuuugugaaagcaacagagaauacagucaauccuugcccagaugauacuuugauaucuuucuugccucucgcccauauguuugagagaguuguagaguguguaaugcugugucauggagcuaaaaucggauuuuuccaaggagauaucaggcugcucauggaugaccucaaggugcuucaacccacugucuuccccgugguuccaagacugcugaaccggauguuugaccgaauuuucggacaagcaaacaccacgcugaagcgauggcucuuggacuuugccuccaagaggaaagaagcagagcuucgcagcggcaucaucagaaacaacagccugugggaccggcugaucuuccacaaaguacagucgagccugggcggaagaguccggcugauggugacaggagccgccccggugucugccacugugcugacguuccucagagcagcccugggcugucaguuuuaugaaggauacggacagacagagugcacugccgggugcugccugaccaugccuggagacuggaccgcaggccauguuggggccccgaugccgugcaauuugauaaaacuuguugauguggaagaaaugaauuacauggcugccgagggcgagggcgaggugugugugaaagggccaaauguauuucagggcuacuugaaggacccagcgaaaacagcagaagcuuuggacaaagacggcugguuacacacaggggacauuggaaaaugguuaccaaauggcaccuugaaaauuaucgaccggaaaaagcacauauuuaagcuggcacaaggagaauacauagccccugaaaagauugaaaauaucuacaugcgaagugagccuguugcucagguguuuguccacggagaaagccugcaggcauuucucauugcaauugugguaccagauguugagacauuauguuccugggcccaaaagagaggauuugaagggucguuugaggaacugugcagaaauaaggaugucaaaaaagcuauccucgaagauauggugagacuugggaaggauucuggucugaaaccauuugaacaggucaaaggcaucacauugcacccugaauuauuuucuaucgacaauggccuucugacuccaacaaugaaggcgaaaaggccagagcugcggaacuauuucaggucgcagauagaugaccucuauuccacuaucaagguuuagugugaagaagaaagcucagaggaaauggcacaguuccacaaucucuucuccugcugauggccuucauguuguuaauuuugaauacagcaaguguagggaaggaagcguucguguuugacuuguccauucgggguucuucucauaggaaugcuagaggaaacagaacacugccuuacagucaccucauguugcagaccauguuuaugguaauacacacuuuccaaaaugagccuuaaaaauuguaaaggggauacuauaaaugugcuaaguuauuugagacuuccucaguuuaaaaaguggguuuuaaaucuucugucucccuguuuuucuaaucaagggguuaggacuuugcuaucucugagaugucugcuacuugcugcaaauucugcagcugucugcugcucuaaagaguacagugcacuagagggaaguguucccuuuaaaaauaagaacaacuguccuggcuggagaaucucacaagcggaccagagaucuuuuuaaaucccugcuacugucccuucucacaggcauucacagaacccuucugauucguaaggguuacgaaacucauguucuucuccaguccccugugguuucuguuggagcauaagguuuccaguaagcgggagggcagauccaacucagaaccaugcagauaaggagccucuggcaaaugggugcucaucagaacgcguggauucucuuucauggcagaaugcucuuggacucgguucuccaggccugauuccccgacuccauccuuuuucagggguuauuuaaaaaucugccuuagauucuauagugaagacaagcauuucaagaaagaguuaccuggaucagccaugcucagcugugacgccugaauaacugucuacuuuaucuucacugaaccacucacucuguguaaaggccaacagauuuuuaaugugguuuucauaucaaaagaucauguugggauuaacuugccuuuuuccccaaaaaauaaacucucaggcaagcauuucuuuaaagcuauuaagggaguauauacuugaguacuuauugaaauggacaguaauaagcaaauguucuuauaaugcuaccugauuucuaugaaauguguuugacaagccaaaauucuaggauguagaaaucuggaaaguucauuuccugggauucacuucuccagggauuuuuuaaaguuaauuugggaaauuaacagcaguucacuuuauugugagucuuugccacauuugacugaauugagcugucauuuguacauuuaaagcagcuguuuuggggucugugagaguacauguauuauauacaagcacaacagggcuugcacuaaagaauugucauuguaauaacacuacuugguagccuaacuucauauauguauucuuaauugcacaaaaagucaauaauuugucaccuugggguuuugaauguuugcuuuaaguguuggcuauuucuauguuuuauaaaccaaaacaaaauuuccaaaaacaaugaaggaaaccaaaauaaauauuucugcauuucaggugaaaaaaaaaaaaaaaaaa

SEQ ID NO: 48 - protein, 698 aa, Homo sapiens
MQAHELFRYFRMPELVDFRQYVRTLPTNTLMGFGAFAALTTFWYATRPKPLKPPCDLSMQSVEVAGSGGARRSALLDSDEPLVYFYDDVTTLYEGFQRGIQVSNNGPCLGSRKPDQPYEWLSYKQVAELSECIGSALIQKGFKTAPDQFIGIFAQNRPEWVIIEQGCFAYSMVIVPLYDTLGNEAITYIVNKAELSLVFVDKPEKAKLLLEGVENKLIPGLKIIVVMDAYGSELVERGQRCGVEVTSMKAMEDLGRANRRKPKPPAPEDLAVICFTSGTTGNPKGAMVTHRNIVSDCSAFVKATENTVNPCPDDTLISFLPLAHMFERVVECVMLCHGAKIGFFQGDIRLLMDDLKVLQPTVFPVVPRLLNRMFDRIFGQANTTLKRWLLDFASKRKEAELRSGIIRNNSLWDRLIFHKVQSSLGGRVRLMVTGAAPVSATVLTFLRAALGCQFYEGYGQTECTAGCCLTMPGDWTAGHVGAPMPCNLIKLVDVEEMNYMAAEGEGEVCVKGPNVFQGYLKDPAKTAEALDKDGWLHTGDIGKWLPNGTLKIIDRKKHIFKLAQGEYIAPEKIENIYMRSEPVAQVFVHGESLQAFLIAIVVPDVETLCSWAQKRGFEGSFEELCRNKDVKKAILEDMVRLGKDSGLKPFEQVKGITLHPELFSIDNGLLTPTMKAKRPELRNYFRSQIDDLYSTIKV